(19) United States
(12) Patent Application Publication
(10) Pub. No.: US 2024/0240207 A1
(43) Pub. Date: Jul. 18, 2024

(54) Increasing Specificity for RNA-Guided Genome Editing

(71) Applicant: The General Hospital Corporation, Boston, MA (US)

(72) Inventors: J. Keith Joung, Winchester, MA (US); James Angstman, Charlestown, MA (US); Shengdar Tsai, Memphis, TN (US)

(21) Appl. No.: 18/415,999

(22) Filed: Jan. 18, 2024

Related U.S. Application Data
(63) Continuation of application No. 17/099,503, filed on Nov. 16, 2020, now Pat. No. 11,920,152.
Application No. 17/099,503 is a continuation of application No. 15/870,659, filed on Jan. 12, 2018, now Pat. No. 10,844,403.
Application No. 15/870,659 is a division of application No. 14/776,620, filed on Sep. 14, 2015, now Pat. No. 9,885,033.
(60) Provisional application No. 61/921,007, filed on Dec. 26, 2013.
(60) Provisional application No. 61/838,178, filed on Jun. 21, 2013.
(60) Provisional application No. 61/838,148, filed on Jun. 21, 2013.
(60) Provisional application No. 61/799,647, filed on Mar. 15, 2013.

(51) Int. Cl. C12N 15/90; C07K 14/005; C07K 14/195; C12N 9/02; C12N 9/10; C12N 9/16; C12N 9/22; C12N 9/96; C12N 15/01; C12N 15/10; C12N 15/11; C12N 15/63; C12N 15/85

(52) U.S. Cl. CPC C12N 15/907; C07K 14/005; C07K 14/195; C12N 9/0071; C12N 9/1007; C12N 9/16; C12N 9/22; C12N 9/96; C12N 15/01; C12N 15/102; C12N 15/1031; C12N 15/11; C12N 15/63; C12N 15/85; C12Y 301/21004; C07K 2319/00; C07K 2319/01; C07K 2319/80; C12N 2310/20; C12N 2710/00033; C12N 2770/00033; C12N 2800/80; C12Y 114/11; C12Y 201/01; C12Y 301/00

(57) ABSTRACT

Methods for increasing specificity of RNA-guided genome editing, e.g., editing using CRISPR/Cas9 systems.

### CLAIM OF PRIORITY

This application is a continuation of U.S. patent application Ser. No. 17/099,503, filed Nov. 16, 2020, which is a continuation of U.S. patent application Ser. No. 15/870,659, filed Jan. 12, 2018, now U.S. Pat. No. 10,844,403, which is a divisional of U.S. patent application Ser. No. 14/776,620, filed Sep. 14, 2015, now U.S. Pat. No. 9,885,033, which is a U.S. National Phase Application under 35 U.S.C. § 371 of International Patent Application No. PCT/US2014/029304, filed on Mar. 14, 2014, which claims priority under 35 USC § 119(e) to U.S. Patent Application Serial Nos. 61/921,007, filed on Dec. 26, 2013: 61/838,178, filed on Jun. 21, 2013; 61/838,148, filed on Jun. 21, 2013; and 61/799,647, filed on Mar. 15, 2013. The entire contents of the foregoing are hereby incorporated by reference.

### FEDERALLY SPONSORED RESEARCH OR DEVELOPMENT

This invention was made with Government support under GM088040, AR063070, GM105378, HG005550, and GM105378 awarded by the National Institutes of Health, and W911NF-11-2-0056 awarded by the Army Research Laboratory—Army Research Office. The Government has certain rights in the invention.

### SEQUENCE LISTING

This application contains a Sequence Listing that has been submitted electronically as an XML file named “40174-0009004_SL_ST26XML.” The XML file, created on Jan. 18, 2024, is 159,275 bytes in size. The material in the XML file is hereby incorporated by reference in its entirety.

### TECHNICAL FIELD

Methods for increasing specificity of RNA-guided genome editing, e.g., editing using CRISPR/Cas9 systems.

### BACKGROUND

Recent work has demonstrated that clustered, regularly interspaced, short palindromic repeats (CRISPR)/CRISPR-associated (Cas) systems (Wiedenheft et al., Nature 482, 331-338 (2012); Horvath et al., Science 327, 167-170 (2010); Terns et al., Curr Opin Microbiol 14, 321-327 (2011)) can serve as the basis for performing genome editing in bacteria, yeast and human cells, as well as in vivo in whole organisms such as fruit flies, zebrafish and mice (Wang et al., Cell 153, 910-918 (2013); Shen et al., Cell Res (2013); Dicarlo et al., Nucleic Acids Res (2013); Jiang et al., Nat Biotechnol 31, 233-239 (2013); Jinek et al., Elife 2, e00471 (2013); Hwang et al., Nat Biotechnol 31, 227-229 (2013); Cong et al., Science 339, 819-823 (2013); Mali et al., Science 339, 823-826 (2013c); Cho et al., Nat Biotechnol 31, 230-232 (2013); Gratz et al., Genetics 194(4); 1029-35 (2013)). The Cas9 nuclease from S. pyogenes (hereafter simply Cas9) can be guided via base pair complementarity between the first 20 nucleotides of an engineered gRNA and the complementary strand of a target genomic DNA sequence of interest that lies next to a protospacer adjacent motif (PAM), e.g., a PAM matching the sequence NGG or NAG (Shen et al., Cell Res (2013); Dicarlo et al., Nucleic Acids Res (2013); Jiang et al., Nat Biotechnol 31, 233-239 (2013); Jinek et al., Elife 2, e00471 (2013); Hwang et al., Nat Biotechnol 31, 227-229 (2013); Cong et al., Science 339, 819-823 (2013); Mali et al., Science 339, 823-826 (2013c); Cho et al., Nat Biotechnol 31, 230-232 (2013); Jinek et al., Science 337, 816-821 (2012)). Previous studies performed in vitro (Jinek et al., Science 337, 816-821 (2012)), in bacteria (Jiang et al., Nat Biotechnol 31, 233-239 (2013)) and in human cells (Cong et al., Science 339, 819-823 (2013)) have shown that Cas9-mediated cleavage can, in some cases, be abolished by single mismatches at the gRNA/target site interface, particularly in the last 10-12 nucleotides (nts) located in the 3′ end of the 20 nt gRNA complementarity region.

### SUMMARY

Studies have shown that CRISPR-Cas nucleases can tolerate up to five mismatches and still cleave: it is hard to predict the effects of any given single or combination of mismatches on activity. Taken together, these nucleases can show significant off-target effects but it can be challenging to predict these sites. Described herein are methods of genome editing using the CRISPR/Cas system, e.g., using Cas9 or Cas9-based fusion proteins.

Thus, in a first aspect, the invention provides a synthetic guide ribonucleic acid, wherein: one or more of the nucleotides is modified, e.g., locked (2′-O-4′-C methylene bridge), is 5′-methylcytidine, is 2′-O-methyl-pseudouridine, or in which the ribose phosphate backbone has been replaced by a polyamide chain; and/or wherein one or more of the nucleotides is a deoxyribonucleic acid.

In one aspect, the invention provides a guide RNA molecule having a target complementarity region of 17-20 nucleotides, e.g., a sequence complementary to the complementary strand of 17-20 consecutive nucleotides of a target sequence, preferably a target sequence immediately 5′ of a protospacer adjacent motif (PAM), e.g., NGG, NAG or NNGG, wherein one or more of the RNA nucleotides is modified, e.g., locked (2′-O-4′-C methylene bridge), is 5′-methylcytidine, is 2′-O-methyl-pseudouridine, or in which the ribose phosphate backbone has been replaced by a polyamide chain, e.g., one or more of the nucleotides within the sequence X₁₇₋₂₀, one or more of the nucleotides within the sequence X_{N}, or one or more of the nucleotides within any sequence of the gRNA. In no case is the X₁₇₋₂₀ identical to a sequence that naturally occurs adjacent to the rest of the RNA. X_{N} is any sequence, wherein N (in the RNA) can be 0-200, e.g., 0-100, 0-50, or 0-20, that does not interfere with the binding of the ribonucleic acid to Cas9. In some embodiments the RNA includes one or more U, e.g., 1 to 8 or more Us (e.g., U, UU, UUU, UUUU, UUUUU, UUUUUU, UUUUUUU, UUUUUUUU) at the 3′ end of the molecule, as a result of the optional presence of one or more Ts used as a termination signal to terminate RNA PolIII transcription. In some embodiments the RNA includes one or more, e.g., up to 3, e.g., one, two, or three, additional nucleotides at the 5′ end of the RNA molecule that is not complementary to the target sequence.

In one aspect, the invention provides a ribonucleic acid comprising or consisting of the sequence:

(SEQ ID NO: 4)
(X₁₇₋₂₀)GUUUUAGAGCUAUGCUGUUUUG(X_{N});
(SEQ ID NO: 5)
(X₁₇₋₂₀)GUUUUAGAGCUA;
(SEQ ID NO: 6)
(X₁₇₋₂₀)GUUUUAGAGCUAUGCUGUUUUG;
(SEQ ID NO: 7)
(X₁₇₋₂₀)GUUUUAGAGCUAUGCU;
(SEQ ID NO: 8)
(X₁₇₋₂₀)GUUUUAGAGCUAGAAAUAGCAAGUUAAAAUAAGGC
UAGUCCG(X_{N});
(SEQ ID NO: 9)
(X₁₇₋₂₀)GUUUUAGAGCUAUGCUGAAAAGCAUAGCAAGUUAA
AAUAAGGCUAGUCCGUUAUC(X_{N});
(SEQ ID NO: 10)
(X₁₇₋₂₀)GUUUUAGAGCUAUGCUGUUUUGGAAACAAAACAGC
AUAGCAAGUUAAAAUAAGGCUAGUCCGUUAUC(X_{N});
(SEQ ID NO: 11)
(X₁₇₋₂₀)GUUUUAGAGCUAGAAAUAGCAAGUUAAAAUAAGGC
UAGUCCGUUAUCAACUUGAAAAAGUGGCACCGAGUCGGUGC
(X_{N}),
(SEQ ID NO: 12)
(X₁₇₋₂₀)GUUUAAGAGCUAGAAAUAGCAAGUUUAAAUAAGGC
UAGUCCGUUAUCAACUUGAAAAAGUGGCACCGAGUCGGUGC;
(SEQ ID NO: 13)
(X₁₇₋₂₀)GUUUUAGAGCUAUGCUGGAAACAGCAUAGCAAGUU
UAAAUAAGGCUAGUCCGUUAUCAACUUGAAAAAGUGGCACC
GAGUCGGUGC;
or
(SEQ ID NO: 14)
(X₁₇₋₂₀)GUUUAAGAGCUAUGCUGGAAACAGCAUAGCAAGUU
UAAAUAAGGCUAGUCCGUUAUCAACUUGAAAAAGUGGCACC
GAGUCGGUGC,

wherein X₁₇₋₂₀ is a sequence complementary to the complementary strand of 17-20 consecutive nucleotides of a target sequence (though in some embodiments this complementarity region may be longer than 20 nts, e.g., 21, 22, 23, 24, 25 or more nts), preferably a target sequence immediately 5′ of a protospacer adjacent motif (PAM), e.g., NGG, NAG or NNGG, wherein one or more of the RNA nucleotides is modified, e.g., locked (2′-O-4′-C methylene bridge), is 5′-methylcytidine, is 2′-O-methyl-pseudouridine, or in which the ribose phosphate backbone has been replaced by a polyamide chain, e.g., one or more of the nucleotides within the sequence X₁₇₋₂₀, one or more of the nucleotides within the sequence X_{N}, or one or more of the nucleotides within any sequence of the gRNA. In no case is the X₁₇₋₂₀ identical to a sequence that naturally occurs adjacent to the rest of the RNA. X_{N} is any sequence, wherein N (in the RNA) can be 0-200, e.g., 0-100, 0-50, or 0-20, that does not interfere with the binding of the ribonucleic acid to Cas9. In some embodiments the RNA includes one or more U, e.g., 1 to 8 or more Us (e.g., U, UU, UUU, UUUU, UUUUU, UUUUUU, UUUUUUU, UUUUUUUU) at the 3′ end of the molecule, as a result of the optional presence of one or more Ts used as a termination signal to terminate RNA PolIII transcription. In some embodiments the RNA includes one or more, e.g., up to 3, e.g., one, two, or three, additional nucleotides at the 5′ end of the RNA molecule that is not complementary to the target sequence.

In another aspect, the invention provides hybrid nucleic acids comprising or consisting of the sequence:
(X₁₇₋₂₀) GUUUUAGAGCUAUGCUGUUUUG(X_{N}) (SEQ ID NO:4);
(X₁₇₋₂₀)GUUUUAGAGCUA (SEQ ID NO:5);
(X₁₇₋₂₀) GUUUUAGAGCUAUGCUGUUUUG (SEQ ID NO:6);
(X₁₇₋₂₀)GUUUUAGAGCUAUGCU (SEQ ID NO:7);
(X₁₇₋₂₀)GUUUUAGAGCUAGAAAUAGCAAGUUAAAAUAAGGCUAGUCCG(X_{N}) (SEQ ID NO:8);
(X₁₇₋₂₀)GUUUUAGAGCUAUGCUGAAAAGCAUAGCAAGUUAAAAUAAGGCU AGUCCGUUAUC(X_{N}) (SEQ ID NO:9);
(X₁₇₋₂₀)GUUUUAGAGCUAUGCUGUUUUGGAAACAAAACAGCAUAGCAAGU UAAAAUAAGGCUAGUCCGUUAUC(X_{N}) (SEQ ID NO:10);
(X₁₇₋₂₀)GUUUUAGAGCUAGAAAUAGCAAGUUAAAAUAAGGCUAGUCCGUU AUCAACUUGAAAAAGUGGCACCGAGUCGGUGC(X_{N}) (SEQ ID NO:11),
(X₁₇₋₂₀)GUUUAAGAGCUAGAAAUAGCAAGUUUAAAUAAGGCUAGUCCGUU AUCAACUUGAAAAAGUGGCACCGAGUCGGUGC(SEQ ID NO:12);
(X₁₇₋₂₀)GUUUUAGAGCUAUGCUGGAAACAGCAUAGCAAGUUUAAAUAAGG CUAGUCCGUUAUCAACUUGAAAAAGUGGCACCGAGUCGGUGC (SEQ ID NO:13); or
(X₁₇₋₂₀)GUUUAAGAGCUAUGCUGGAAACAGCAUAGCAAGUUUAAAUAAGG CUAGUCCGUUAUCAACUUGAAAAAGUGGCACCGAGUCGGUGC (SEQ ID NO:14), wherein the X₁₇₋₂₀ is a sequence complementary to the complementary strand of 17-20 consecutive nucleotides of a target sequence (though in some embodiments this complementarity region may be longer than 20 nts, e.g., 21, 22, 23, 24, 25 or more nts), preferably a target sequence immediately 5′ of a protospacer adjacent motif (PAM), e.g., NGG NAG or NNGG, wherein the nucleic acid is at least partially or wholly DNA, or is partially RNA and partially DNA. In no case is the X₁₇₋₂₀ identical to a sequence that naturally occurs adjacent to the rest of the RNA. X_{N} is any sequence, wherein N (in the RNA) can be 0-200, e.g., 0-100, 0-50, or 0-20, that does not interfere with the binding of the ribonucleic acid to Cas9. In some embodiments the RNA includes one or more U, e.g., 1 to 8 or more Us (e.g., U, UU, UUU, UUUU, UUUUU, UUUUUU, UUUUUUU, UUUUUUUU) at the 3′ end of the molecule, as a result of the optional presence of one or more Ts used as a termination signal to terminate RNA PolIII transcription. In some embodiments the RNA includes one or more, e.g., up to 3, e.g., one, two, or three, additional nucleotides at the 5′ end of the RNA molecule that is not complementary to the target sequence.

In another aspect, the invention provides DNA molecules encoding the ribonucleic acids described herein.

In yet another aspect, the invention provides methods for inducing a single or double-stranded break in a target region of a double-stranded DNA molecule, e.g., in a genomic sequence in a cell. The methods include expressing in or introducing into the cell: a Cas9 nuclease or nickase; and
(a) a guide RNA that includes one or more deoxyribonuclotides (e.g., where the sequence may also be partially or wholly DNA but with thymine in place or uracil), e.g., a guide RNA that includes a sequence of 17-20 nucleotides that are complementary to the complementary strand of a target sequence, preferably a target sequence immediately 5′ of a protospacer adjacent motif (PAM), e.g., NGG, NAG, or NNGG, wherein the guide RNA includes one or more deoxyribonuclotides (e.g., where the defined sequence may also be partially or wholly DNA but with thymine in place or uracil), e.g., a hybrid nucleic acid as described herein; or
(b) a guide RNA wherein one or more of the nucleotides is modified, e.g., locked (2′-O-4′-C methylene bridge), is 5′-methylcytidine, is 2′-O-methyl-pseudouridine, or in which the ribose phosphate backbone has been replaced by a polyamide chain, e.g., a guide RNA that includes a sequence of 17-20 nucleotides that are complementary to a target sequence, preferably a target sequence immediately 5′ of a protospacer adjacent motif (PAM), e.g., NGG NAG or NNGG, wherein one or more of the nucleotides is modified, e.g., locked (2′-O-4′-C methylene bridge), is 5′-methylcytidine, is 2′-O-methyl-pseudouridine, or in which the ribose phosphate backbone has been replaced by a polyamide chain, e.g., a ribonucleic acid as described herein.

In yet another aspect, the invention provides methods for inducing a single or double-stranded break in a target region of a double-stranded DNA molecule, e.g., in a genomic sequence in a cell. The methods include expressing in or introducing into the cell:
a Cas9 nuclease or nickase:
a tracrRNA, e.g., comprising the sequence of
GUUUUAGAGCUAGAAAUAGCAAGUUAAAAUAAGGCUAGUCCGUUAUCA ACUUGAAAAAGUGGCACCGAGTCGGUGCUUUU (SEQ ID NO:15) or an active portion thereof,
UAGCAAGUUAAAAUAAGGCUAGUCCGUUAUCAACUUGAAAAAGUGGCA CCGAGUCGGUGC (SEQ ID NO: 16) or an active portion thereof:
AGCAUAGCAAGUUAAAAUAAGGCUAGUCCGUUAUCAACUUGAAAAAGU GGCACCGAGUCGGUGC (SEQ ID NO:17) or an active portion thereof, GGAACCAUUCAAAACAGCAUAGCAAGUUAAAAUAAGGCUAGUCCGUUA UCAACUUGAAAAAGUGGCACCGAGUCGGUGC (SEQ ID NO:42) or an active portion thereof:
UAGCAAGUUAAAAUAAGGCUAGUCCGUUAUCAACUUGAAAAAGUGGCA CCGAGUCGGUGC (SEQ ID NO:16) or an active portion thereof: CAAAACAGCAUAGCAAGUUAAAAUAAGGCUAGUCCGUUAUCAACUUGA AAAAGUGGCACCGAGUCGGUGC (SEQ ID NO:43) or an active portion thereof: AGCAUAGCAAGUUAAAAUAAGGCUAGUCCGUUAUCAACUUGAAAAAGU
GGCACCGAGUCGGUGC (SEQ ID NO:17) or an active portion thereof: UAGCAAGUUAAAAUAAGGCUAGUCCGUUAUCAACUUGAAAAAGUG (SEQ ID NO:44) or an active portion thereof: UAGCAAGUUAAAAUAAGGCUAGUCCGUUAUCA (SEQ ID NO:45) or an active portion thereof; or
UAGCAAGUUAAAAUAAGGCUAGUCCG (SEQ ID NO:45) or an active portion thereof: and
(a) a crRNA that includes or more deoxyribonuclotides (e.g., wherein the sequence may also be partially or wholly DNA but with thymine in place or uracil), e.g., wherein the target complementarity region is at least partially or wholly DNA, e.g., a crRNA that includes a sequence of 17-20 nucleotides that are complementary to a target sequence, preferably a target sequence immediately 5′ of a protospacer adjacent motif (PAM), e.g., NGG, NAG, or NNGG, wherein the crRNA includes one or more deoxyribonuclotides (e.g., where the defined sequence may also be partially or wholly DNA but with thymine in place or uracil), e.g., wherein the crRNA consists of the sequence: 5′-X₁₇₋₂₀GUUUUAGAGCUAUGCUGUUUUG(X_{N})-3′ (SEQ ID NO:46); (X₁₇₋₂₀)GUUUUAGAGCUA (SEQ ID NO:5);
(X₁₇₋₂₀)GUUUUAGAGCUAUGCUGUUUUG (SEQ ID NO:6); or
(X₁₇₋₂₀)GUUUUAGAGCUAUGCU (SEQ ID NO:7); where the X₁₇₋₂₀ is at least partially or wholly DNA and is a sequence complementary to 17-20 consecutive nucleotides of a target sequence; or
(b) a crRNA that includes one or more nucleotides that are modified, e.g., locked (2′-O-4′-C methylene bridge), is 5′-methylcytidine, is 2′-O-methyl-pseudouridine, or in which the ribose phosphate backbone has been replaced by a polyamide chain, e.g., wherein one or more of the nucleotides in the target complementarity region is modified, e.g., a crRNA that includes a sequence of 17-20 nucleotides that are complementary to a target sequence, preferably a target sequence immediately 5′ of a protospacer adjacent motif (PAM), e.g., NGG, NAG, or NNGG, wherein one or more of the nucleotides is modified, e.g., locked (2′-O-4′-C methylene bridge), is 5′-methylcytidine, is 2′-O-methyl-pseudouridine, or in which the ribose phosphate backbone has been replaced by a polyamide chain, e.g., wherein the crRNA consists of the sequence: 5′-X₁₇₋₂₀GUUUUAGAGCUAUGCUGUUUUG(X_{N})-3′ (SEQ ID NO:46); (X₁₇₋₂₀)GUUUUAGAGCUA (SEQ ID NO:5);
(X₁₇₋₂₀)GUUUUAGAGCUAUGCUGUUUUG (SEQ ID NO:6); or
(X₁₇₋₂₀)GUUUUAGAGCUAUGCU (SEQ ID NO:7); where one or more of the X₁₇₋₂₀ wherein one or more of the nucleotides is modified, e.g., locked (2′-O-4′-C methylene bridge), is 5′-methylcytidine, is 2′-O-methyl-pseudouridine, or in which the ribose phosphate backbone has been replaced by a polyamide chain. In no case is the X₁₇₋₂₀ identical to a sequence that naturally occurs adjacent to the rest of the RNA. In some embodiments the RNA includes one or more U, e.g., 1 to 8 or more Us (e.g., U, UU, UUU, UUUU, UUUUU, UUUUUU, UUUUUUU, UUUUUUUU) at the 3′ end of the molecule, as a result of the optional presence of one or more Ts used as a termination signal to terminate RNA PolIII transcription. In some embodiments the RNA includes one or more, e.g., up to 3, e.g., one, two, or three, additional nucleotides at the 5′ end of the RNA molecule that is not complementary to the target sequence.

In some embodiments wherein (X₁₇₋₂₀)GUUUUAGAGCUAUGCUGUUUUG (SEQ ID NO:6) is used as a crRNA, the following tracrRNA is used: GGAACCAUUCAAAACAGCAUAGCAAGUUAAAAUAAGGCUAGUCCGUUA UCAACUUGAAAAAGUGGCACCGAGUCGGUGC (SEQ ID NO:8) or an active portion thereof. In some embodiments wherein (X₁₇₋₂₀)GUUUUAGAGCUA (SEQ ID NO:5) is used as a crRNA, the following tracrRNA is used: UAGCAAGUUAAAAUAAGGCUAGUCCGUUAUCAACUUGAAAAAGUGGCA CCGAGUCGGUGC (SEQ ID NO:16) or an active portion thereof. In some embodiments wherein (X₁₇₋₂₀) GUUUUAGAGCUAUGCU (SEQ ID NO:4) is used as a crRNA, the following tracrRNA is used: AGCAUAGCAAGUUAAAAUAAGGCUAGUCCGUUAUCAACUUGAAAAAGU GGCACCGAGUCGGUGC (SEQ ID NO:17) or an active portion thereof.

In yet another aspect, the invention provides methods for sequence-specifically inducing a pair of nicks in a double-stranded DNA molecule, e.g., in a genomic sequence in a cell. The methods include expressing in the cell, or introducing into the cell or contacting the cell with, a Cas9-nickase as known in the art or described herein, and:
(a) two guide RNAs, wherein one of the two guide RNAs includes sequence that is complementary to one strand of the target sequence and the second of the two guide RNAS includes sequence that is complementary to the other strand of the target sequence, such that using both guide RNAs results in targeting both strands, and the Cas9-nickase results in cuts being introduced into each strand; or
(b) a tracrRNA and two crRNAs wherein one of the two crRNAs includes sequence that is complementary to one strand of the target sequence and the second of the two crRNAs is complementary to the other strand of the target sequence, such that using both crRNAs results in targeting both strands, and the Cas9-nickase cuts each strand.

In some embodiments, the method includes contacting the cell with two nickases, wherein the first nickase comprises a Cas9 with a mutation at D10, E762, H983, or D986 and the second nickase comprises a Cas9 with a mutation at H840 or N863.

In some embodiments wherein (X₁₇₋₂₀)GUUUUAGAGCUAUGCUGUUUUG (SEQ ID NO:6) is used as a crRNA, the following tracrRNA is used: GGAACCAUUCAAAACAGCAUAGCAAGUUAAAAUAAGGCUAGUCCGUUA UCAACUUGAAAAAGUGGCACCGAGUCGGUGC (SEQ ID NO:8) or an active portion thereof. In some embodiments wherein (X₁₇₋₂₀)GUUUUAGAGCUA (SEQ ID NO:5) is used as a crRNA, the following tracrRNA is used: UAGCAAGUUAAAAUAAGGCUAGUCCGUUAUCAACUUGAAAAAGUGGCA CCGAGUCGGUGC (SEQ ID NO:16) or an active portion thereof. In some embodiments wherein (X₁₇₋₂₀) GUUUUAGAGCUAUGCU (SEQ ID NO:4) is used as a crRNA, the following tracrRNA is used: AGCAUAGCAAGUUAAAAUAAGGCUAGUCCGUUAUCAACUUGAAAAAGU GGCACCGAGUCGGUGC (SEQ ID NO:17) or an active portion thereof.

In an additional aspect, the invention provides three-part fusion guide nucleic acid comprising, in any order that preserves activity of each part: (1) a first sequence that is complementary to the complementary strand of a target genomic sequence, e.g., a first sequence of 17-20 or 17-25 consecutive nucleotides that is complementary to 17-20 or 17-25 consecutive nucleotides of the complementary strand of a target sequence: (2) a second sequence comprising all or part of a Cas9 guide RNA that forms a stem-loop sequence that is recognized by and binds to Cas9; and (3) a third sequence that binds to an RNA binding protein, e.g., MS2, CRISPR/Cas Subtype Ypest protein 4 (Csy4), or lambda N. In some embodiments, the first and second sequences comprise:
(X₁₇₋₂₀) GUUUUAGAGCUAUGCUGUUUUG(X_{N}) (SEQ ID NO:4);
(X₁₇₋₂₀)GUUUUAGAGCUA (SEQ ID NO:5);
(X₁₇₋₂₀) GUUUUAGAGCUAUGCUGUUUUG (SEQ ID NO:6);
(X₁₇₋₂₀)GUUUUAGAGCUAUGCU (SEQ ID NO:7);
(X₁₇₋₂₀)GUUUUAGAGCUAGAAAUAGCAAGUUAAAAUAAGGCUAGUCCG(X_{N}) (SEQ ID NO:8);
(X₁₇₋₂₀)GUUUUAGAGCUAUGCUGAAAAGCAUAGCAAGUUAAAAUAAGGCU AGUCCGUUAUC(X_{N}) (SEQ ID NO:9);
(X₁₇₋₂₀)GUUUUAGAGCUAUGCUGUUUUGGAAACAAAACAGCAUAGCAAGU UAAAAUAAGGCUAGUCCGUUAUC(X_{N}) (SEQ ID NO:10);
(X₁₇₋₂₀)GUUUUAGAGCUAGAAAUAGCAAGUUAAAAUAAGGCUAGUCCGUU AUCAACUUGAAAAAGUGGCACCGAGUCGGUGC(X_{N}) (SEQ ID NO:11),
(X₁₇₋₂₀)GUUUAAGAGCUAGAAAUAGCAAGUUUAAAUAAGGCUAGUCCGUU AUCAACUUGAAAAAGUGGCACCGAGUCGGUGC(SEQ ID NO: 12);
(X₁₇₋₂₀)GUUUUAGAGCUAUGCUGGAAACAGCAUAGCAAGUUUAAAUAAGG CUAGUCCGUUAUCAACUUGAAAAAGUGGCACCGAGUCGGUGC (SEQ ID NO:13); or
(X₁₇₋₂₀)GUUUAAGAGCUAUGCUGGAAACAGCAUAGCAAGUUUAAAUAAGG CUAGUCCGUUAUCAACUUGAAAAAGUGGCACCGAGUCGGUGC (SEQ ID NO:14), wherein X₁₇₋₂₀ is a sequence complementary to 17-20 nts of a target sequence. In no case is the X_{N} identical to a sequence that naturally occurs adjacent to the rest of the RNA. X_{N} is any sequence, wherein N (in the RNA) can be 0-200, e.g., 0-100, 0-50, or 0-20, that does not interfere with the binding of the ribonucleic acid to Cas9. In some embodiments the RNA includes one or more U, e.g., 1 to 8 or more Us (e.g., U, UU, UUU, UUUU, UUUUU, UUUUUU, UUUUUUU, UUUUUUUU) at the 3′ end of the molecule, as a result of the optional presence of one or more Ts used as a termination signal to terminate RNA PolIII transcription. In some embodiments the RNA includes one or more, e.g., up to 3, e.g., one, two, or three, additional nucleotides at the 5′ end of the RNA molecule that is not complementary to the target sequence.

In yet another aspect, the invention provides tracrRNA molecule comprising a sequence GUUUUAGAGCUAGAAAUAGCAAGUUAAAAUAAGGCUAGUCCGUUAUCA ACUUGAAAAAGUGGCACCGAGTCGGUGCUUUU (SEQ ID NO:15) or an active portion thereof, UAGCAAGUUAAAAUAAGGCUAGUCCGUUAUCAACUUGAAAAAGUGGCA CCGAGUCGGUGC (SEQ ID NO: 16) or an active portion thereof; or AGCAUAGCAAGUUAAAAUAAGGCUAGUCCGUUAUCAACUUGAAAAAGU GGCACCGAGUCGGUGC (SEQ ID NO:17) or an active portion thereof, linked to a sequence that binds to an RNA binding protein, e.g., MS2, Csy4 (e.g., GUUCACUGCCGUAUAGGCAG or GUUCACUGCCGUAUAGGCAGCUAAGAAA), or lambda N. In some embodiments, the tracrRNA molecule may be truncated from its 3′ end by at least 1, 2, 3, 4, 5, 6, 7, 8, 9, 10, 15, 20, 25, 30, 35 or 40 nts. In another embodiment, the tracrRNA molecule may be truncated from its 5′ end by at least 1, 2, 3, 4, 5, 6, 7, 8, 9, 10, 15, 20, 25, 30, 35 or 40 nts. Alternatively, the tracrRNA molecule may be truncated from both the 5′ and 3′ end, e.g., by at least 1, 2, 3, 4, 5, 6, 7, 8, 9, 10, 15 or 20 nts on the 5′ end and at least 1, 2, 3, 4, 5, 6, 7, 8, 9, 10, 15, 20, 25, 30, 35 or 40 nts on the 3′ end. Additional exemplary tracrRNA sequences include: GGAACCAUUCAAAACAGCAUAGCAAGUUAAAAUAAGGCUAGUCCGUUA UCAACUUGAAAAAGUGGCACCGAGUCGGUGC (SEQ ID NO:42) or an active portion thereof: UAGCAAGUUAAAAUAAGGCUAGUCCGUUAUCAACUUGAAAAAGUGGCA CCGAGUCGGUGC (SEQ ID NO:16) or an active portion thereof: CAAAACAGCAUAGCAAGUUAAAAUAAGGCUAGUCCGUUAUCAACUUGA AAAAGUGGCACCGAGUCGGUGC (SEQ ID NO:43) or an active portion thereof: AGCAUAGCAAGUUAAAAUAAGGCUAGUCCGUUAUCAACUUGAAAAAGU GGCACCGAGUCGGUGC (SEQ ID NO:17) or an active portion thereof: UAGCAAGUUAAAAUAAGGCUAGUCCGUUAUCAACUUGAAAAAGUG (SEQ ID NO:44) or an active portion thereof: UAGCAAGUUAAAAUAAGGCUAGUCCGUUAUCA (SEQ ID NO:45) or an active portion thereof; or UAGCAAGUUAAAAUAAGGCUAGUCCG (SEQ ID NO:45) or an active portion thereof.

In some embodiments the RNA includes one or more U, e.g., 1 to 8 or more Us (e.g., U, UU, UUU, UUUU, UUUUU, UUUUUU, UUUUUUU, UUUUUUUU) at the 3′ end of the molecule, as a result of the optional presence of one or more Ts used as a termination signal to terminate RNA PolIII transcription. In some embodiments the RNA includes one or more, e.g., up to 3, e.g., one, two, or three, additional nucleotides at the 5′ end of the RNA molecule that is not complementary to the target sequence.

In some embodiments wherein (X₁₇₋₂₀)GUUUUAGAGCUAUGCUGUUUUG (SEQ ID NO:6) is used as a crRNA, the following tracrRNA is used: GGAACCAUUCAAAACAGCAUAGCAAGUUAAAAUAAGGCUAGUCCGUUA UCAACUUGAAAAAGUGGCACCGAGUCGGUGC (SEQ ID NO:8) or an active NO:5) is used as a crRNA, the following tracrRNA is used: UAGCAAGUUAAAAUAAGGCUAGUCCGUUAUCAACUUGAAAAAGUGGCA CCGAGUCGGUGC (SEQ ID NO: 16) or an active portion thereof. In some embodiments wherein (X₁₇₋₂₀) GUUUUAGAGCUAUGCU (SEQ ID NO:4) is used as a crRNA, the following tracrRNA is used: AGCAUAGCAAGUUAAAAUAAGGCUAGUCCGUUAUCAACUUGAAAAAGU GGCACCGAGUCGGUGC (SEQ ID NO:17) or an active portion thereof.

In another aspect, the invention provides DNA molecules encoding the three-part fusion guide nucleic acids or the tracrRNA described herein.

In yet another aspect, the invention provides fusion proteins comprising an RNA binding protein, e.g., MS2, Csy4, or lambda N, linked to a catalytic domain of a FokI nuclease or to a heterologous functional domain (HFD) as described herein, optionally with an intervening linker of 2-30, e.g., 5-20 nts, and DNA molecules encoding the fusion proteins. In some embodiment, the fusion protein comprises a FokI catalytic domain sequence fused to the N terminus of Csy4, with an intervening linker, optionally a linker of from 2-30 amino acids, e.g., 4-12 amino acids, e.g., Gly4Ser, (Gly4Ser)1-5. In some embodiments the HFD modifies gene expression, histones, or DNA, e.g., transcriptional activation domain, transcriptional repressors (e.g., silencers such as Heterochromatin Protein 1 (HP1), e.g., HPla or HPIB, or a transcriptional repression domain, e.g., Krueppel-associated box (KRAB) domain, ERF repressor domain (ERD), or mSin3A interaction domain (SID)), enzymes that modify the methylation state of DNA (e.g., DNA methyltransferase (DNMT) or Ten-Eleven Translocation (TET) proteins, e.g., TET1, also known as Tet Methylcytosine Dioxygenase 1), or enzymes that modify histone subunit (e.g., histone acetyltransferases (HAT), histone deacetylases (HDAC), or histone methyltransferase or histone demethylases).

In a further aspect, the invention provides methods for sequence-specifically inducing a break in a double-stranded DNA molecule, e.g., in a genomic sequence in a cell. The methods include expressing in the cell, or contacting the cell with a fusion protein comprising an RNA binding protein, e.g., MS2, Csy4, or lambda N, linked to a catalytic domain of a FokI nuclease, optionally with an intervening linker of 2-30, e.g., 5-20 nts,
a dCas9 protein: and
(a) a three-part fusion guide nucleic acid described herein,
(b) a tracrRNA as described herein, and a crRNA suitable for use with the tracrRNA: and/or
(c) a DNA molecule encoding a three-part fusion guide nucleic acid or tracrRNA as described herein.

In yet another aspect, the invention provides vectors comprising the DNA molecules described herein, and host cells expressing the vectors.

In an additional aspect, the invention provides methods for modifying a target region of a double-stranded DNA molecule, e.g., in a genomic sequence in a cell. The methods include expressing in or introducing into the cell:
a dCas9-heterologous functional domain fusion protein (dCas9-HFD); and
(a) a guide RNA that includes one or more deoxyribonuclotides (e.g., where the sequence may also be partially or wholly DNA but with thymine in place or uracil), e.g., a guide RNA that includes a sequence of 17-20 nucleotides that are complementary to the complementary strand of a target sequence, preferably a target sequence immediately 5′ of a protospacer adjacent motif (PAM), e.g., NGG NAG or NNGG, wherein the guide RNA includes one or more deoxyribonuclotides (e.g., where the defined sequence may also be partially or wholly DNA but with thymine in place or uracil), e.g., hybrid nucleic acid as described herein; or
(b) a guide RNA wherein one or more of the nucleotides is modified, e.g., locked (2′-O-4′-C methylene bridge), is 5′-methylcytidine, is 2′-O-methyl-pseudouridine, or in which the ribose phosphate backbone has been replaced by a polyamide chain, e.g., a guide RNA that includes a sequence of 17-20 nucleotides that are complementary to the complementary strand of a target sequence, preferably a target sequence immediately 5′ of a protospacer adjacent motif (PAM), e.g., NGG NAG or NNGG, wherein one or more of the nucleotides is modified, e.g., locked (2′-O-4′-C methylene bridge), is 5′-methylcytidine, is 2′-O-methyl-pseudouridine, or in which the ribose phosphate backbone has been replaced by a polyamide chain, e.g., a synthetic ribonucleic acid as described herein. In no case is the X₁₇₋₂₀ identical to a sequence that naturally occurs adjacent to the rest of the RNA. In some embodiments the RNA includes one or more, e.g., up to 3, e.g., one, two, or three, additional nucleotides at the 5′ end of the RNA molecule that is not complementary to the target sequence.

In another aspect, the invention provides methods for modifying a target region of a double-stranded DNA molecule, e.g., in a genomic sequence in a cell. The methods include expressing in or introducing into the cell:
a dCas9-heterologous functional domain fusion protein (dCas9-HFD);
a tracrRNA, e.g., comprising the sequence of tracrRNA molecule comprising a sequence
GUUUUAGAGCUAGAAAUAGCAAGUUAAAAUAAGGCUAGUCCGUUAUCA ACUUGAAAAAGUGGCACCGAGTCGGUGCUUUU (SEQ ID NO:15) or an active portion thereof,
UAGCAAGUUAAAAUAAGGCUAGUCCGUUAUCAACUUGAAAAAGUGGCA CCGAGUCGGUGC (SEQ ID NO: 16) or an active portion thereof; or
AGCAUAGCAAGUUAAAAUAAGGCUAGUCCGUUAUCAACUUGAAAAAGU GGCACCGAGUCGGUGC (SEQ ID NO:17) or an active portion thereof; and
(a) a crRNA that includes or more deoxyribonuclotides (e.g., wherein the sequence may also be partially or wholly DNA but with thymine in place or uracil), e.g., wherein the target complementarity region is at least partially or wholly DNA, e.g., a crRNA that includes a sequence of 17-20 nucleotides that are complementary to the complementary strand of a target sequence, preferably a target sequence immediately 5′ of a protospacer adjacent motif (PAM), e.g., NGG NAG or NNGG, wherein the crRNA includes one or more deoxyribonuclotides (e.g., where the defined sequence may also be partially or wholly DNA but with thymine in place or uracil), e.g., wherein the crRNA consists of the sequence:
5′-X₁₇₋₂₀GUUUUAGAGCUAUGCUGUUUUG(X_{N})-3′ (SEQ ID NO:46);
(X₁₇₋₂₀)GUUUUAGAGCUA (SEQ ID NO:5);
(X₁₇₋₂₀)GUUUUAGAGCUAUGCUGUUUUG (SEQ ID NO:6); or
(X₁₇₋₂₀)GUUUUAGAGCUAUGCU (SEQ ID NO:7); where the X₁₇₋₂₀ is at least partially or wholly DNA and is a sequence complementary to 17-20 consecutive nucleotides of a target sequence; or
(b) a crRNA that includes one or more nucleotides that are modified, e.g., locked (2′-O-4′-C methylene bridge), is 5′-methylcytidine, is 2′-O-methyl-pseudouridine, or in which the ribose phosphate backbone has been replaced by a polyamide chain, e.g., wherein one or more of the nucleotides in the target complementarity region is modified, e.g., a crRNA that includes a sequence of 17-20 nucleotides that are complementary to the complementary strand of a target sequence, preferably a target sequence immediately 5′ of a protospacer adjacent motif (PAM), e.g., NGG NAG, or NNGG, wherein one or more of the nucleotides is modified, e.g., locked (2′-O-4′-C methylene bridge), is 5′-methylcytidine, is 2′-O-methyl-pseudouridine, or in which the ribose phosphate backbone has been replaced by a polyamide chain, e.g., wherein the crRNA consists of the sequence:
5′-X₁₇₋₂₀GUUUUAGAGCUAUGCUGUUUUG(X_{N})-3′ (SEQ ID NO:46);
(X₁₇₋₂₀)GUUUUAGAGCUA (SEQ ID NO:5);
(X₁₇₋₂₀)GUUUUAGAGCUAUGCUGUUUUG (SEQ ID NO:6); or
(X₁₇₋₂₀)GUUUUAGAGCUAUGCU (SEQ ID NO:7); where one or more of the X₁₇₋₂₀ wherein one or more of the nucleotides is modified, e.g., locked (2′-O-4′-C methylene bridge), is 5′-methylcytidine, is 2′-O-methyl-pseudouridine, or in which the ribose phosphate backbone has been replaced by a polyamide chain. In no case is the X₁₇₋₂₀ identical to a sequence that naturally occurs adjacent to the rest of the RNA. In some embodiments the RNA includes one or more U, e.g., 1 to 8 or more Us (e.g., U, UU, UUU, UUUU, UUUUU, UUUUUU, UUUUUUU, UUUUUUUU) at the 3′ end of the molecule, as a result of the optional presence of one or more Ts used as a termination signal to terminate RNA PolIII transcription. In some embodiments the RNA includes one or more, e.g., up to 3, e.g., one, two, or three, additional nucleotides at the 5′ end of the RNA molecule that is not complementary to the target sequence.

In some embodiments, the dCas9-heterologous functional domain fusion protein (dCas9-HFD) comprises a HFD that modifies gene expression, histones, or DNA, e.g., transcriptional activation domain, transcriptional repressors (e.g., silencers such as Heterochromatin Protein 1 (HP1), e.g., HPla or HPIB, or a transcriptional repression domain, e.g., Krueppel-associated box (KRAB) domain, ERF repressor domain (ERD), or mSin3A interaction domain (SID)), enzymes that modify the methylation state of DNA (e.g., DNA methyltransferase (DNMT) or Ten-Eleven Translocation (TET) proteins, e.g., TET1, also known as Tet Methylcytosine Dioxygenase 1), or enzymes that modify histone subunit (e.g., histone acetyltransferases (HAT), histone deacetylases (HDAC), or histone methyltransferase or histone demethylases). In some embodiments, the heterologous functional domain is a transcriptional activation domain, e.g., a transcriptional activation domain from VP64 or NF-κB p65: an enzyme that catalyzes DNA demethylation, e.g., a TET; or histone modification (e.g., LSD1, histone methyltransferase, HDACs, or HATs) or a transcription silencing domain, e.g., from Heterochromatin Protein 1 (HP1), e.g., HP1α or HP1β; or a biological tether, e.g., CRISPR/Cas Subtype Ypest protein 4 (Csy4), MS2, or lambda N protein. Cas9-HFD are described in a U.S. Provisional Patent Applications U.S. Ser. No. 61/799,647, Filed on Mar. 15, 2013, U.S. Ser. No. 61/838,148, filed on Jun. 21, 2013, and PCT International Application No. PCT/US14/27335, all of which are incorporated herein by reference in its entirety.

Unless otherwise defined, all technical and scientific terms used herein have the same meaning as commonly understood by one of ordinary skill in the art to which this invention belongs. Methods and materials are described herein for use in the present invention: other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1: Schematic illustrating a gRNA/Cas9 nuclease complex bound to its target DNA site. Scissors indicate approximate cleavage points of the Cas9 nuclease on the genomic DNA target site. Note the numbering of nucleotides on the guide RNA proceeds in an inverse fashion from 5′ to 3′.

FIG. 2A: Schematic illustrating the rationale for truncating the 5′ complementarity region of a gRNA. Thick black lines=target DNA site, line structure=gRNA, grey oval=Cas9 nuclease, black lines indicate base pairing between gRNA and target DNA site.

FIG. 2B: Schematic overview of the EGFP disruption assay. Repair of targeted Cas9-mediated double-stranded breaks in a single integrated EGFP-PEST reporter gene by error-prone NHEJ-mediated repair leads to frame-shift mutations that disrupt the coding sequence and associated loss of fluorescence in cells.

FIGS. 2C-F: Activities of CRISPR RNA-guided nucleases (RGNs) with gRNAs bearing (C) single mismatches, (D) adjacent double mismatches, (E) variably spaced double mismatches, and (F) increasing numbers of adjacent mismatches assayed on three different target sites in the EGFP reporter gene sequence. Mean activities of replicates (see Online Methods) are shown, normalized to the activity of a perfectly matched gRNA. Error bars indicate standard errors of the mean. Positions mismatched in each gRNA are highlighted in grey in the grid below. Sequences of the three EGFP target sites were as follows:

EGFP Site 1
SEQ ID NO: 1
GGGCACGGGCAGCTTGCCGGTGG
EGFP Site 2
SEQ ID NO: 2
GATGCCGTTCTTCTGCTTGTCGG
EGFP Site 3
SEQ ID NO: 3
GGTGGTGCAGATGAACTTCAGGG

FIG. 2G: Mismatches at the 5′ end of the gRNA make CRISPR/Cas more sensitive more 3′ mismatches. The gRNAs Watson-Crick base pair between the RNA&DNA with the exception of positions indicated with an “m” which are mismatched using the Watson-Crick transversion (i.e. EGFP Site #2 M18-19 is mismatched by changing the gRNA to its Watson-Crick partner at positions 18 & 19. Although positions near the 5′ of the gRNA are generally very well tolerated, matches in these positions are important for nuclease activity when other residues are mismatched. When all four positions are mismatched, nuclease activity is no longer detectable. This further demonstrates that matches at these 5′ position can help compensate for mismatches at other more 3′ positions. Note these experiments were performed with a non-codon optimized version of Cas9 which can show lower absolute levels of nuclease activity as compared to the codon optimized version.

FIG. 2H: Efficiency of Cas9 nuclease activities directed by gRNAs bearing variable length complementarity regions ranging from 15 to 25 nts in a human cell-based U2OS EGFP disruption assay. Expression of a gRNA from the U6 promoter requires the presence of a 5′ G and therefore it was only possible to evaluate gRNAs harboring certain lengths of complementarity to the target DNA site (15, 17, 19, 20, 21, 23, and 25 nts).

FIG. 3A: Efficiencies of EGFP disruption in human cells mediated by Cas9 and full-length or shortened gRNAs for four target sites in the EGFP reporter gene. Lengths of complementarity regions and corresponding target DNA sites are shown. Ctrl=control gRNA lacking a complementarity region.

FIG. 3B: Efficiencies of targeted indel mutations introduced at seven different human endogenous gene targets by matched standard and tru-RGNs. Lengths of complementarity regions and corresponding target DNA sites are shown. Indel frequencies were measured by T7EI assay. Ctrl=control gRNA lacking a complementarity region.

FIG. 3C: DNA sequences of indel mutations induced by RGNs using a tru-gRNA or a matched full-length gRNA targeted to the EMX1 site. The portion of the target DNA site that interacts with the gRNA complementarity region is highlighted in grey with the first base of the PAM sequence shown in lowercase. Deletions are indicated by dashes highlighted in grey and insertions by italicized letters highlighted in grey. The net number of bases deleted or inserted and the number of times each sequence was isolated are shown to the right.

FIG. 3D: Efficiencies of precise HDR/ssODN-mediated alterations introduced at two endogenous human genes by matched standard and tru-RGNs. % HDR was measured using a BamHI restriction digest assay (see the Experimental Procedures for Example 2). Control gRNA=empty U6 promoter vector.

FIG. 3E: U2OS.EGFP cells were transfected with variable amounts of full-length gRNA expression plasmids (top) or tru-gRNA expression plasmids (bottom) together with a fixed amount of Cas9 expression plasmid and then assayed for percentage of cells with decreased EGFP expression. Mean values from duplicate experiments are shown with standard errors of the mean. Note that the data obtained with tru-gRNA matches closely with data from experiments performed with full-length gRNA expression plasmids instead of tru-gRNA plasmids for these three EGFP target sites.

FIG. 3F: U2OS.EGFP cells were transfected with variable amount of Cas9 expression plasmid together with variable amounts of full-length gRNA expression plasmids (top) or tru-gRNA expression plasmids (bottom) (amounts determined for each tru-gRNA from the experiments of FIG. 3E). Mean values from duplicate experiments are shown with standard errors of the mean. Note that the data obtained with tru-gRNA matches closely with data from experiments performed with full-length gRNA expression plasmids instead of tru-gRNA plasmids for these three EGFP target sites. The results of these titrations determined the concentrations of plasmids used in the EGFP disruption assays performed in Examples 1 and 2.

FIG. 4: Schematic representation of gRNA-guided RGN and DNA-guided Cas9 nuclease. The gRNA fusion RNA molecule can bind to both its on-target sequence (no asterisks) and a wide range of off-target sites (mismatches denoted by asterisks) and induce DNA cleavage. Because of the increased sensitivity of DNA-DNA duplexes to mismatches, a DNA-guided Cas9 nuclease system that uses a short DNA oligonucleotide with complementarity to a tracRNA may no longer be able to bind and cut at off-target sites, but may still function in genomic localization of Cas9. This may lead to a marked increase in Cas9-mediated nuclease activity over traditional RGNs.

FIG. 5: Pairs of Cas9 RNA-guided nickases used to create paired nicks on opposing strands of DNA

FIG. 6: Schematic illustrating recruitment of two RNA-binding protein-FokI nuclease domain fusions to the DNA (see text for details).

FIG. 7A: Variant gRNAs bearing a Csy4 binding site can function to recruit Cas9 to specific sites in human cells.

FIG. 7B: Three-part complex of catalytically inactive Cas9 nuclease (dCas9), gRNA with Csy4 recognition site, and FokI-Csy4 fusion. Protospacer adjacent motif (PAM) sequences are facing ‘outward’ in this configuration.

FIG. 7C: dCas9/gRNA/FokI-Csy4 pairs with spacer lengths of 15-16 bp showing the highest level of activity in an EGFP-disruption assay.

FIG. 7D: T7 endonuclease I assay showing molecular evidence of non-homologous end joining-mediated DNA double-stranded break repair in dCas9/gRNA/FokI-Csy4 treated samples, but not in negative controls.

### DETAILED DESCRIPTION

CRISPR RNA-guided nucleases (RGNs) have rapidly emerged as a facile and efficient platform for genome editing. Although Marraffini and colleagues (Jiang et al., Nat Biotechnol 31, 233-239 (2013)) recently performed a systematic investigation of Cas9 RGN specificity in bacteria, the specificities of RGNs in human cells have not been extensively defined. Understanding the scope of RGN-mediated off-target effects in human and other eukaryotic cells will be critically essential if these nucleases are to be used widely for research and therapeutic applications. The present inventors have used a human cell-based reporter assay to characterize off-target cleavage of Cas9-based RGNs. Single and double mismatches were tolerated to varying degrees depending on their position along the guide RNA (gRNA)-DNA interface. Off-target alterations induced by four out of six RGNs targeted to endogenous loci in human cells were readily detected by examination of partially mismatched sites. The off-target sites identified harbor up to five mismatches and many are mutagenized with frequencies comparable to (or higher than) those observed at the intended on-target site. Thus RGNs are highly active even with imperfectly matched RNA-DNA interfaces in human cells, a finding that might confound their use in research and therapeutic applications.

The results described herein reveal that predicting the specificity profile of any given RGN is neither simple nor straightforward. The EGFP reporter assay experiments show that single and double mismatches can have variable effects on RGN activity in human cells that do not strictly depend upon their position(s) within the target site. For example, consistent with previously published reports, alterations in the 3′ half of the gRNA/DNA interface generally have greater effects than those in the 5′ half (Jiang et al., Nat Biotechnol 31, 233-239 (2013); Cong et al., Science 339, 819-823 (2013); Jinek et al., Science 337, 816-821 (2012)); however, single and double mutations in the 3′ end sometimes also appear to be well tolerated whereas double mutations in the 5′ end can greatly diminish activities. In addition, the magnitude of these effects for mismatches at any given position(s) appears to be site-dependent. Comprehensive profiling of a large series of RGNs with testing of all possible nucleotide substitutions (beyond the Watson-Crick transversions used in our EGFP reporter experiments) may help provide additional insights into the range of potential off-targets. In this regard, the recently described bacterial cell-based method of Marraffini and colleagues (Jiang et al., Nat Biotechnol 31, 233-239 (2013)) or the in vitro, combinatorial library-based cleavage site-selection methodologies previously applied to ZFNs by Liu and colleagues (Pattanayak et al., Nat Methods 8, 765-770 (2011)) might be useful for generating larger sets of RGN specificity profiles.

Despite these challenges in comprehensively predicting RGN specificities, it was possible to identify bona fide off-targets of RGNs by examining a subset of genomic sites that differed from the on-target site by one to five mismatches. Notably, under conditions of these experiments, the frequencies of RGN-induced mutations at many of these off-target sites were similar to (or higher than) those observed at the intended on-target site, enabling the detection of mutations at these sites using the T7EI assay (which, as performed in our laboratory, has a reliable detection limit of ˜2 to 5% mutation frequency). Because these mutation rates were very high, it was possible to avoid using deep sequencing methods previously required to detect much lower frequency ZFN- and TALEN-induced off-target alterations (Pattanayak et al., Nat Methods 8, 765-770 (2011); Perez et al., Nat Biotechnol 26, 808-816 (2008); Gabriel et al., Nat Biotechnol 29, 816-823 (2011); Hockemeyer et al., Nat Biotechnol 29, 731-734 (2011)). Analysis of RGN off-target mutagenesis in human cells also confirmed the difficulties of predicting RGN specificities—not all single and double mismatched off-target sites show evidence of mutation whereas some sites with as many as five mismatches can also show alterations. Furthermore, the bona fide off-target sites identified do not exhibit any obvious bias toward transition or transversion differences relative to the intended target sequence.

Although off-target sites were seen for a number of RGNs, identification of these sites was neither comprehensive nor genome-wide in scale. For the six RGNs studied, only a very small subset of the much larger total number of potential off-target sequences in the human genome (sites that differ by three to six nucleotides from the intended target site) was examined. Although examining such large numbers of loci for off-target mutations by T7EI assay is neither a practical nor a cost-effective strategy, the use of high-throughput sequencing in future studies might enable the interrogation of larger numbers of candidate off-target sites and provide a more sensitive method for detecting bona fide off-target mutations. For example, such an approach might enable the unveiling of additional off-target sites for the two RGNs for which we failed to uncover any off-target mutations. In addition, an improved understanding both of RGN specificities and of any epigenomic factors (e.g., DNA methylation and chromatin status) that may influence RGN activities in cells might also reduce the number of potential sites that need to be examined and thereby make genome-wide assessments of RGN off-targets more practical and affordable.

As described herein, a number of strategies can be used to minimize the frequencies of genomic off-target mutations. For example, the specific choice of RGN target site can be optimized: given that off-target sites that differ at up to five positions from the intended target site can be efficiently mutated by RGNs, choosing target sites with minimal numbers of off-target sites as judged by mismatch counting seems unlikely to be effective: thousands of potential off-target sites that differ by four or five positions within the 20 bp RNA:DNA complementarity region will typically exist for any given RGN targeted to a sequence in the human genome. It is also possible that the nucleotide content of the gRNA complementarity region might influence the range of potential off-target effects. For example, high GC-content has been shown to stabilize RNA:DNA hybrids (Sugimoto et al., Biochemistry 34, 11211-11216 (1995)) and therefore might also be expected to make gRNA/genomic DNA hybridization more stable and more tolerant to mismatches. Additional experiments with larger numbers of gRNAs will be needed to assess if and how these two parameters (numbers of mismatched sites in the genome and stability of the RNA:DNA hybrid) influence the genome-wide specificities of RGNs. However, it is important to note that even if such predictive parameters can be defined, the effect of implementing such guidelines would be to further restrict the targeting range of RGNs.

One potential general strategy for reducing RGN-induced off-target effects might be to reduce the concentrations of gRNA and Cas9 nuclease expressed in the cell. This idea was tested using the RGNs for VEGFA target sites 2 and 3 in U2OS.EGFP cells: transfecting less gRNA- and Cas9-expressing plasmid decreased the mutation rate at the on-target site but did not appreciably change the relative rates of off-target mutations. Consistent with this, high-level off-target mutagenesis rates were also observed in two other human cell types (HEK293 and K562 cells) even though the absolute rates of on-target mutagenesis are lower than in U2OS.EGFP cells. Thus, reducing expression levels of gRNA and Cas9 in cells is not likely to provide a solution for reducing off-target effects. Furthermore, these results also suggest that the high rates of off-target mutagenesis observed in human cells are not caused by overexpression of gRNA and/or Cas9.

The finding that significant off-target mutagenesis can be induced by RGNs in three different human cell types has important implications for broader use of this genome-editing platform. For research applications, the potentially confounding effects of high frequency off-target mutations will need to be considered, particularly for experiments involving either cultured cells or organisms with slow generation times for which the outcrossing of undesired alterations would be challenging. One way to control for such effects might be to utilize multiple RGNs targeted to different DNA sequences to induce the same genomic alteration because off-target effects are not random but instead related to the targeted site. However, for therapeutic applications, these findings clearly indicate that the specificities of RGNs will need to be carefully defined and/or improved if these nucleases are to be used safely in the longer term for treatment of human diseases.

### Methods for Improving Specificity

As shown herein, CRISPR-Cas RNA-guided nucleases based on the S. pyogenes Cas9 protein can have significant off-target mutagenic effects that are comparable to or higher than the intended on-target activity (Example 1). Such off-target effects can be problematic for research and in particular for potential therapeutic applications. Therefore, methods for improving the specificity of CRISPR-Cas RNA guided nucleases (RGNs) are needed.

As described in Example 1, Cas9 RGNs can induce high-frequency indel mutations at off-target sites in human cells (see also Cradick et al., 2013; Fu et al., 2013: Hsu et al., 2013; Pattanayak et al., 2013). These undesired alterations can occur at genomic sequences that differ by as many as five mismatches from the intended on-target site (see Example 1). In addition, although mismatches at the 5′ end of the gRNA complementarity region are generally better tolerated than those at the 3′ end, these associations are not absolute and show site-to-site-dependence (see Example 1 and Fu et al., 2013: Hsu et al., 2013; Pattanayak et al., 2013). As a result, computational methods that rely on the number and/or positions of mismatches currently have limited predictive value for identifying bona fide off-target sites. Therefore, methods for reducing the frequencies of off-target mutations remain an important priority if RNA-guided nucleases are to be used for research and therapeutic applications.

Strategy #1: Synthetic Alternatives to Standard gRNAs to Improve Specificity

Guide RNAs generally speaking come in two different systems: System 1, which uses separate crRNA and tracrRNAs that function together to guide cleavage by Cas9, and System 2, which uses a chimeric crRNA-tracrRNA hybrid that combines the two separate guide RNAs in a single system (referred to as a single guide RNA or sgRNA, see also Jinek et al., Science 2012: 337:816-821). The tracrRNA can be variably truncated and a range of lengths has been shown to function in both the separate system (system 1) and the chimeric gRNA system (system 2). For example, in some embodiments, tracrRNA may be truncated from its 3′ end by at least 1, 2, 3, 4, 5, 6, 7, 8, 9, 10, 15, 20, 25, 30, 35 or 40 nts. In some embodiments, the tracrRNA molecule may be truncated from its 5′ end by at least 1, 2, 3, 4, 5, 6, 7, 8, 9, 10, 15, 20, 25, 30, 35 or 40 nts. Alternatively, the tracrRNA molecule may be truncated from both the 5′ and 3′ end, e.g., by at least 1, 2, 3, 4, 5, 6, 7, 8, 9, 10, 15 or 20 nts on the 5′ end and at least 1, 2, 3, 4, 5, 6, 7, 8, 9, 10, 15, 20, 25, 30, 35 or 40 nts on the 3′ end. See, e.g., Jinek et al., Science 2012: 337:816-821: Mali et al., Science. 2013 Feb 15:339(6121); 823-6; Cong et al., Science. 2013 Feb 15:339(6121); 819-23; and Hwang and Fu et al., Nat Biotechnol. 2013 Mar:31(3);227-9; Jinek et al., Elife 2, e00471 (2013)). For System 2, generally the longer length chimeric gRNAs have shown greater on-target activity but the relative specificities of the various length gRNAs currently remain undefined and therefore it may be desirable in certain instances to use shorter gRNAs. In some embodiments, the gRNAs are complementary to a region that is within about 100-800 bp upstream of the transcription start site, e.g., is within about 500 bp upstream of the transcription start site, includes the transcription start site, or within about 100-800 bp, e.g., within about 500 bp, downstream of the transcription start site. In some embodiments, vectors (e.g., plasmids) encoding more than one gRNA are used, e.g., plasmids encoding, 2, 3, 4, 5, or more gRNAs directed to different sites in the same region of the target gene.

Described herein are guide RNAs, e.g., single gRNAs or crRNA and tracrRNA, that include one or more modified (e.g., locked) nucleotides or deoxyribonucleotides.

### Strategy 1A: Modified Nucleic Acid Molecules

Modified RNA oligonucleotides such as locked nucleic acids (LNAs) have been demonstrated to increase the specificity of RNA-DNA hybridization by locking the modified oligonucleotides in a more favorable (stable) conformation. For example, 2′-O-methyl RNA is a modified base where there is an additional covalent linkage between the 2′ oxygen and 4′ carbon which when incorporated into oligonucleotides can improve overall thermal stability and selectivity (formula I).

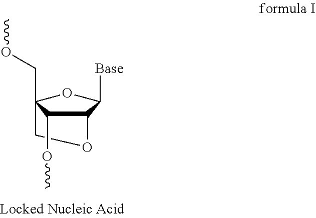

Guide RNAs as described herein may be synthetic guide RNA molecules wherein one, some or all of the nucleotides 5′ region of the guide RNA complementary to the target sequence are modified, e.g., locked (2′-O-4′-C methylene bridge), 5′-methylcytidine, 2′-O-methyl-pseudouridine, or in which the ribose phosphate backbone has been replaced by a polyamide chain (peptide nucleic acid), e.g., a synthetic ribonucleic acid.

In another embodiment, one, some or all of the nucleotides of the gRNA sequence may be modified, e.g., locked (2′-O-4′-C methylene bridge), 5′-methylcytidine, 2′-O-methyl-pseudouridine, or in which the ribose phosphate backbone has been replaced by a polyamide chain (peptide nucleic acid), e.g., a synthetic ribonucleic acid.

In a cellular context, complexes of Cas9 with these synthetic gRNAs could be used to improve the genome-wide specificity of the CRISPR/Cas9 nuclease system. Exemplary modified or synthetic gRNAs may comprise, or consist of, the following sequences:
(X₁₇₋₂₀) GUUUUAGAGCUAUGCUGUUUUG(X_{N}) (SEQ ID NO:4);
(X₁₇₋₂₀)GUUUUAGAGCUA (SEQ ID NO:5);
(X₁₇₋₂₀) GUUUUAGAGCUAUGCUGUUUUG (SEQ ID NO:6);
(X₁₇₋₂₀)GUUUUAGAGCUAUGCU (SEQ ID NO:7);
(X₁₇₋₂₀)GUUUUAGAGCUAGAAAUAGCAAGUUAAAAUAAGGCUAGUCCG(X_{N}) (SEQ ID NO:8);
(X₁₇₋₂₀)GUUUUAGAGCUAUGCUGAAAAGCAUAGCAAGUUAAAAUAAGGCU AGUCCGUUAUC(X_{N}) (SEQ ID NO:9);
(X₁₇₋₂₀)GUUUUAGAGCUAUGCUGUUUUGGAAACAAAACAGCAUAGCAAGU UAAAAUAAGGCUAGUCCGUUAUC(X_{N}) (SEQ ID NO:10);
(X₁₇₋₂₀)GUUUUAGAGCUAGAAAUAGCAAGUUAAAAUAAGGCUAGUCCGUU AUCAACUUGAAAAAGUGGCACCGAGUCGGUGC(X_{N}) (SEQ ID NO:11),
(X₁₇₋₂₀)GUUUAAGAGCUAGAAAUAGCAAGUUUAAAUAAGGCUAGUCCGUU AUCAACUUGAAAAAGUGGCACCGAGUCGGUGC(SEQ ID NO:12);
(X₁₇₋₂₀)GUUUUAGAGCUAUGCUGGAAACAGCAUAGCAAGUUUAAAUAAGG CUAGUCCGUUAUCAACUUGAAAAAGUGGCACCGAGUCGGUGC (SEQ ID NO:13); or
(X₁₇₋₂₀)GUUUAAGAGCUAUGCUGGAAACAGCAUAGCAAGUUUAAAUAAGG CUAGUCCGUUAUCAACUUGAAAAAGUGGCACCGAGUCGGUGC (SEQ ID NO:14), wherein X₁₇₋₂₀ is a sequence complementary to 17-20 nts of a target sequence, preferably a target sequence immediately 5′ of a protospacer adjacent motif (PAM), e.g., NGG, NAG, or NNGG, and further wherein one or more of the nucleotides are locked, e.g., one or more of the nucleotides within the sequence X₁₇₋₂₀, one or more of the nucleotides within the sequence X_{N}, or one or more of the nucleotides within any sequence of the gRNA. In some embodiments, X₁₇₋₂₀ is X₁₇₋₁₈, e.g., is 17-18 nucleotides long: in some embodiments, the target complementarity can be longer, e.g., 17-20, 21, 22, 23, 24, 25, or more nucleotides long. X_{N} is any sequence, wherein N (in the RNA) can be 0-200, e.g., 0-100, 0-50, or 0-20, that does not interfere with the binding of the ribonucleic acid to Cas9. In some embodiments the RNA includes one or more U, e.g., 1 to 8 or more Us (e.g., U, UU, UUU, UUUU, UUUUU, UUUUUU, UUUUUUU, UUUUUUUU) at the 3′ end of the molecule, as a result of the optional presence of one or more Ts used as a termination signal to terminate RNA PolIII transcription.

In addition, in a system that uses separate crRNA and tracrRNA, one or both can be synthetic and include one or more locked nucleotides, as dual gRNAs (e.g., the crRNA and tracrRNA found in naturally occurring systems) can also be modified. In this case, a single tracrRNA would be used in conjunction with multiple different crRNAs expressed using the present system, e.g., the following:
(X₁₇₋₂₀)GUUUUAGAGCUA (SEQ ID NO:5);
(X₁₇₋₂₀)GUUUUAGAGCUAUGCUGUUUUG (SEQ ID NO:6); or
(X₁₇₋₂₀)GUUUUAGAGCUAUGCU (SEQ ID NO:7); and a tracrRNA sequence. In this case, the crRNA is used as the guide RNA in the methods and molecules described herein, and the tracrRNA can be expressed from the same or a different DNA molecule. In some embodiments, the methods include contacting the cell with a tracrRNA comprising or consisting of the sequence GGAACCAUUCAAAACAGCAUAGCAAGUUAAAAUAAGGCUAGUCCGUUA UCAACUUGAAAAAGUGGCACCGAGUCGGUGC (SEQ ID NO:15) or an active portion thereof (an active portion is one that retains the ability to form complexes with Cas9 or dCas9). In some embodiments, the tracrRNA molecule may be truncated from its 3′ end by at least 1, 2, 3, 4, 5, 6, 7, 8, 9, 10, 15, 20, 25, 30, 35 or 40 nts. In another embodiment, the tracrRNA molecule may be truncated from its 5′ end by at least 1, 2, 3, 4, 5, 6, 7, 8, 9, 10, 15, 20, 25, 30, 35 or 40 nts. Alternatively, the tracrRNA molecule may be truncated from both the 5′ and 3′ end, e.g., by at least 1, 2, 3, 4, 5, 6, 7, 8, 9, 10, 15 or 20 nts on the 5′ end and at least 1, 2, 3, 4, 5, 6, 7, 8, 9, 10, 15, 20, 25, 30, 35 or 40 nts on the 3′ end. Exemplary tracrRNA sequences in addition to SEQ ID NO:8 include the following: UAGCAAGUUAAAAUAAGGCUAGUCCGUUAUCAACUUGAAAAAGUGGCA CCGAGUCGGUGC (SEQ ID NO: 16) or an active portion thereof; or AGCAUAGCAAGUUAAAAUAAGGCUAGUCCGUUAUCAACUUGAAAAAGU GGCACCGAGUCGGUGC (SEQ ID NO:17) or an active portion thereof.

In some embodiments wherein (X₁₇₋₂₀)GUUUUAGAGCUAUGCUGUUUUG (SEQ ID NO:6) is used as a crRNA, the following tracrRNA is used: GGAACCAUUCAAAACAGCAUAGCAAGUUAAAAUAAGGCUAGUCCGUUA UCAACUUGAAAAAGUGGCACCGAGUCGGUGC (SEQ ID NO:15) or an active NO:5) is used as a crRNA, the following tracrRNA is used: UAGCAAGUUAAAAUAAGGCUAGUCCGUUAUCAACUUGAAAAAGUGGCA CCGAGUCGGUGC (SEQ ID NO: 16) or an active portion thereof. In some embodiments wherein (X₁₇₋₂₀)GUUUUAGAGCUAUGCU (SEQ ID NO:7) is used as a crRNA, the following tracrRNA is used: AGCAUAGCAAGUUAAAAUAAGGCUAGUCCGUUAUCAACUUGAAAAAGU GGCACCGAGUCGGUGC (SEQ ID NO:17) or an active portion thereof.

In a system that uses separate crRNA and tracrRNA, one or both can be synthetic and include one or more modified (e.g., locked) nucleotides.

In some embodiments, the single guide RNAs or crRNAs or tracrRNAs includes one or more Adenine (A) or Uracil (U) nucleotides on the 3′ end.

The methods described can include contacting the cell with a locked gRNA as described herein, and contacting the cell with or expressing in the cell a nuclease that can be guided by the locked gRNAs, e.g., a Cas9 nuclease, e.g., as described in Mali et al., a Cas9 nickase as described in Jinek et al., 2012; or a dCas9-heterofunctional domain fusion (dCas9-HFD) as described in U.S. Provisional Patent Applications U.S. Ser. No. 61/799,647, Filed on Mar. 15, 2013, USSN 61/838,148, filed on Jun. 21, 2013, and PCT International Application No. PCT/US14/27335, all of which are incorporated herein by reference in its entirety.

### Strategy 1B: DNA-Based Guide Molecules

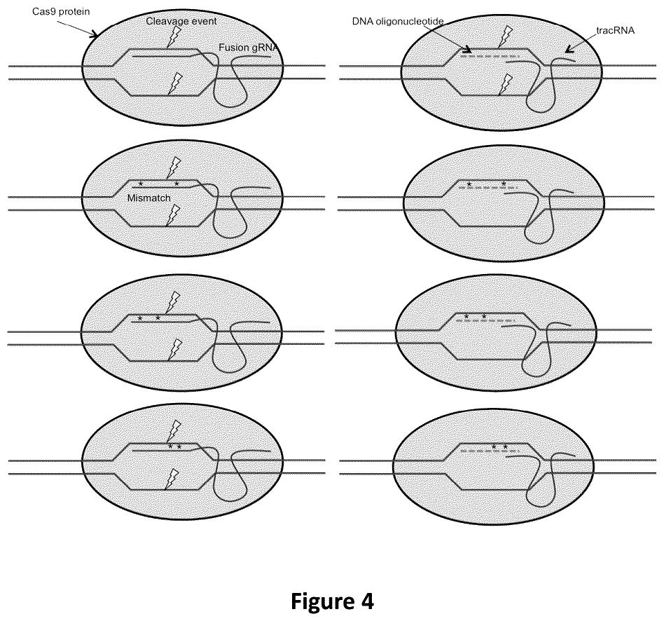

Existing Cas9-based RGNs use gRNA-DNA heteroduplex formation to guide targeting to genomic sites of interest. However, RNA-DNA heteroduplexes can form a more promiscuous range of structures than their DNA-DNA counterparts. In effect, DNA-DNA duplexes are more sensitive to mismatches, suggesting that a DNA-guided nuclease may not bind as readily to off-target sequences, making them comparatively more specific than RNA-guided nucleases. To this end, we propose an engineered Cas9-based RGN wherein a short DNA oligonucleotide replaces all or part of the complementarity region of a gRNA (for example, see FIG. 4). This DNA-based molecule could replace either all or part of the gRNA in a single gRNA system or alternatively might replace all of part of the crRNA in a dual crRNA/tracrRNA system. Such a system that incorporates DNA into the complementarity region should more reliably target the intended genomic DNA sequences due to the general intolerance of DNA-DNA duplexes to mismatching compared to RNA-DNA duplexes. Methods for making such duplexes are known in the art, See, e.g., Barker et al., BMC Genomics. 2005 Apr 22:6:57: and Sugimoto et al., Biochemistry. 2000 Sep 19:39(37); 11270-81. Thus, in some embodiments, described herein are hybrid guide DNA/RNAs consisting of the sequence:
(X₁₇₋₂₀) GUUUUAGAGCUAUGCUGUUUUG(X_{N}) (SEQ ID NO:4);
(X₁₇₋₂₀)GUUUUAGAGCUA (SEQ ID NO:5);
(X₁₇₋₂₀) GUUUUAGAGCUAUGCUGUUUUG (SEQ ID NO:6);
(X₁₇₋₂₀)GUUUUAGAGCUAUGCU (SEQ ID NO:7);
(X₁₇₋₂₀)GUUUUAGAGCUAGAAAUAGCAAGUUAAAAUAAGGCUAGUCCG(X_{N}) (SEQ ID NO:8);
(X₁₇₋₂₀)GUUUUAGAGCUAUGCUGAAAAGCAUAGCAAGUUAAAAUAAGGCU AGUCCGUUAUC(X_{N}) (SEQ ID NO:9);
(X₁₇₋₂₀)GUUUUAGAGCUAUGCUGUUUUGGAAACAAAACAGCAUAGCAAGU UAAAAUAAGGCUAGUCCGUUAUC(X_{N}) (SEQ ID NO:10);
(X₁₇₋₂₀)GUUUUAGAGCUAGAAAUAGCAAGUUAAAAUAAGGCUAGUCCGUU AUCAACUUGAAAAAGUGGCACCGAGUCGGUGC(X_{N}) (SEQ ID NO:11),
(X₁₇₋₂₀)GUUUAAGAGCUAGAAAUAGCAAGUUUAAAUAAGGCUAGUCCGUU AUCAACUUGAAAAAGUGGCACCGAGUCGGUGC(SEQ ID NO: 12);
(X₁₇₋₂₀)GUUUUAGAGCUAUGCUGGAAACAGCAUAGCAAGUUUAAAUAAGG CUAGUCCGUUAUCAACUUGAAAAAGUGGCACCGAGUCGGUGC (SEQ ID NO:13); or
(X₁₇₋₂₀)GUUUAAGAGCUAUGCUGGAAACAGCAUAGCAAGUUUAAAUAAGG CUAGUCCGUUAUCAACUUGAAAAAGUGGCACCGAGUCGGUGC (SEQ ID NO:14), wherein the X₁₇₋₂₀ is a sequence complementary to 17-20 nts of a target sequence, preferably a target sequence immediately 5′ of a protospacer adjacent motif (PAM), e.g., NGG, NAG, or NNGG, wherein the X₁₇₋₂₀ is at least partially or wholly DNA, e.g., one or more of the nucleotides are deoxyribonucleotides (e.g., is all or partially DNA, e.g. DNA/RNA hybrids), e.g., one or more of the nucleotides within the sequence X₁₇₋₂₀, one or more of the nucleotides within the sequence X_{N}, or one or more of the nucleotides within any sequence of the gRNA is a deoxyribonucleotide. In some embodiments, X₁₇₋₂₀ is X₁₇₋₁₈, e.g., is 17-18 nucleotides long. X_{N} is any sequence, wherein N (in the RNA) can be 0-200, e.g., 0-100, 0-50, or 0-20, that does not interfere with the binding of the ribonucleic acid to Cas9. In some embodiments the RNA includes one or more U, e.g., 1 to 8 or more Us (e.g., U, UU, UUU, UUUU, UUUUU, UUUUUU, UUUUUUU, UUUUUUUU) at the 3′ end of the molecule, as a result of the optional presence of one or more Ts used as a termination signal to terminate RNA PolIII transcription.

In addition, in a system that uses separate crRNA and tracrRNA, one or both can be synthetic and include one or more deoxyribonucleotides, as dual gRNAs (e.g., the crRNA and tracrRNA found in naturally occurring systems) can also be hybrids. In this case, a single tracrRNA would be used in conjunction with multiple different crRNAs expressed using the present system, e.g., the following:
(X₁₇₋₂₀)GUUUUAGAGCUA (SEQ ID NO:5);
(X₁₇₋₂₀)GUUUUAGAGCUAUGCUGUUUUG (SEQ ID NO:6); or
(X₁₇₋₂₀)GUUUUAGAGCUAUGCU (SEQ ID NO:7); and a tracrRNA sequence. In this case, the crRNA is used as the guide RNA in the methods and molecules described herein, and the tracrRNA can be expressed from the same or a different DNA molecule. In some embodiments, the methods include contacting the cell with a tracrRNA comprising or consisting of the sequence GGAACCAUUCAAAACAGCAUAGCAAGUUAAAAUAAGGCUAGUCCGUUA UCAACUUGAAAAAGUGGCACCGAGUCGGUGC (SEQ ID NO:15) or an active portion thereof (an active portion is one that retains the ability to form complexes with Cas9 or dCas9). In some embodiments, the tracrRNA molecule may be truncated from its 3′ end by at least 1, 2, 3, 4, 5, 6, 7, 8, 9, 10, 15, 20, 25, 30, 35 or 40 nts. In another embodiment, the tracrRNA molecule may be truncated from its 5′ end by at least 1, 2, 3, 4, 5, 6, 7, 8, 9, 10, 15, 20, 25, 30, 35 or 40 nts. Alternatively, the tracrRNA molecule may be truncated from both the 5′ and 3′ end, e.g., by at least 1, 2, 3, 4, 5, 6, 7, 8, 9, 10, 15 or 20 nts on the 5′ end and at least 1, 2, 3, 4, 5, 6, 7, 8, 9, 10, 15, 20, 25, 30, 35 or 40 nts on the 3′ end. Exemplary tracrRNA sequences in addition to SEQ ID NO:8 include the following: UAGCAAGUUAAAAUAAGGCUAGUCCGUUAUCAACUUGAAAAAGUGGCA CCGAGUCGGUGC (SEQ ID NO: 16) or an active portion thereof; or AGCAUAGCAAGUUAAAAUAAGGCUAGUCCGUUAUCAACUUGAAAAAGU GGCACCGAGUCGGUGC (SEQ ID NO:17) or an active portion thereof.

In some embodiments wherein (X₁₇₋₂₀)GUUUUAGAGCUAUGCUGUUUUG (SEQ ID NO:6) is used as a crRNA, the following tracrRNA is used: GGAACCAUUCAAAACAGCAUAGCAAGUUAAAAUAAGGCUAGUCCGUUA UCAACUUGAAAAAGUGGCACCGAGUCGGUGC (SEQ ID NO: 15) or an active portion thereof. In some embodiments wherein (X₁₇₋₂₀)GUUUUAGAGCUA (SEQ ID NO:5) is used as a crRNA, the following tracrRNA is used: UAGCAAGUUAAAAUAAGGCUAGUCCGUUAUCAACUUGAAAAAGUGGCA CCGAGUCGGUGC (SEQ ID NO: 16) or an active portion thereof. In some embodiments wherein (X₁₇₋₂₀)GUUUUAGAGCUAUGCU (SEQ ID NO:7) is used as a crRNA, the following tracrRNA is used: AGCAUAGCAAGUUAAAAUAAGGCUAGUCCGUUAUCAACUUGAAAAAGU GGCACCGAGUCGGUGC (SEQ ID NO:17) or an active portion thereof.

In a system that uses separate crRNA and tracrRNA, one or both can be synthetic and include one or more deoxyribonucleotides.

In some embodiments, the guide RNA includes one or more Adenine (A) or Uracil (U) nucleotides on the 3′ end. In some embodiments the RNA includes one or more U, e.g., 1 to 8 or more Us (e.g., U, UU, UUU, UUUU, UUUUU, UUUUUU, UUUUUUU, UUUUUUUU) at the 3′ end of the molecule, as a result of the optional presence of one or more Ts used as a termination signal to terminate RNA PolIII transcription.

### Strategy #2: Use of Pairs of Cas9 RNA-Guided Nickases (RGNickases) to Induce Paired Nicks on Opposing Strands of DNA

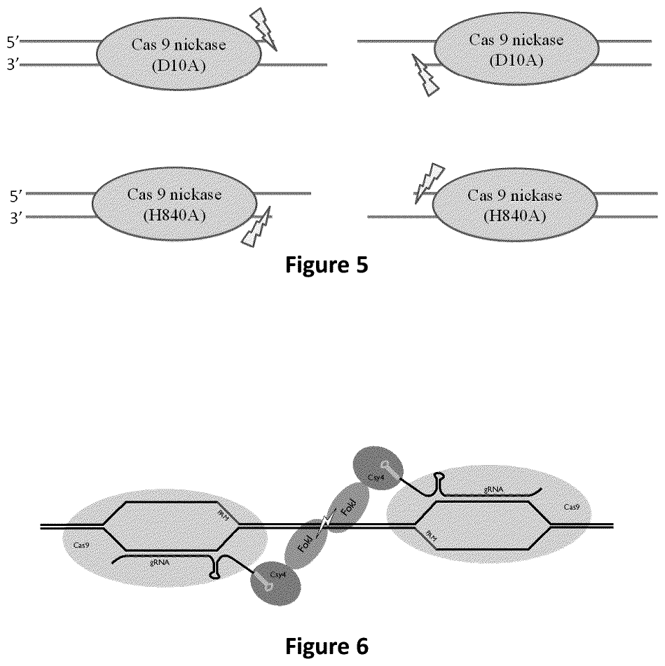

Mutations have been described that inactivate one of the two endonuclease activities found in the S. pyogenes Cas9 nuclease (Jinek et al., Science 2012: Nishimasu al., Cell 156, 935-949 (2014)). Introduction of one of these mutations converts an RGN into an RGNickase that cuts only one of the two DNA strands in a predictable fashion (Jinek et al., Science 2012). Thus by using pairs of appropriately placed RGNickases (two gRNAs and one Cas9 nickase), one can introduce targeted paired nicks on opposing strands of DNA (FIG. 5). Depending on the positioning of these RGNickases and which strand is cleaved by each of them, one can imagine that these nicks might be positioned on opposing strands in one orientation or another (FIG. 5). Because two nickases result in a doubling in the target length this can lead to greater specificity.

In some embodiments, the present system utilizes the Cas9 protein from S. pyogenes, either as encoded in bacteria or codon-optimized for expression in mammalian cells, containing mutations the nuclease portion of the protein partially catalytically inactive. The wild type sequence of the S. pyogenes Cas9 that can be used in the methods and compositions described herein is set forth below.

Thus described herein are methods that include expressing in a cell, or contacting a cell with, two guide RNAs and one Cas9-nickase (e.g., a Cas9 with a mutation at any of D10, E762, H983, D986, H840, or N863, that renders only one of the nuclease portions of the protein catalytically inactive: substitutions at these positions could be alanine (as they are in Nishimasu al., Cell 156, 935-949 (2014)) or they could be other residues, e.g., glutamine, asparagine, tyrosine, serine, or aspartate, e.gE762Q, H983N/H983Y, D986N, N863D/N863S/N863H D10A/DION, H840A/H840N/H840Y), wherein each of the two guide RNAs include sequences that are complementary to either strand of the target sequence, such that using both guide RNAs results in targeting both strands, and the Cas9-nickase cuts each strand singly on opposing strands of DNA. The RGNickase, like RGNs consisting of wildtype Cas9 is expected to cut the DNA target site approximately 3 bp upstream of the PAM, with the D10A Cas9 cleaving the complementary DNA strand and the H840A Cas9 cleaving the non-complementary strand. The two gRNA target sites may be overlapping or some distance away from each other, e.g., up to about 200 nts apart, e.g., less than 100, 50, 25, 10, 5, 4, or 2 nts apart.

### Strategy #3: RNA-Binding Protein-FokI/HFD Fusions

Another method to improve the specificity of Cas9 is to use dCas9 together with a modified gRNA bearing extra RNA sequence on either the 5′ or 3′ end of the gRNA (or on the ends of the crRNA and/or tracrRNA if using a dual gRNA system) that is bound by an RNA-binding protein that is in turn fused to a heterologous functional domain (HFD), e.g., the FokI nuclease domain. In this configuration (FIG. 6), two dCas9 molecules would be targeted to adjacent DNA sequences by appropriate gRNAs and the “extra” RNA sequence on the two gRNA would interact with an appropriate RNA-binding protein-HFD (e.g., FokI nuclease domain) fusion. In the appropriate configuration, the HFD/FokI nuclease domains would dimerize, thereby resulting in introduction of a targeted double-stranded break in the DNA sequence between the two dCas9 binding sites. In addition to the example described herein of FokI-Csy4, VP64-Csy4, TET1-Csy4, and so on could be used. As with the strategy described above, this would result in the need to use two modified gRNAs to form the complex having dCas9 and the required RNA-binding protein-FokI domain fusion molecules, thereby requiring greater specificity than that of a single gRNA-Cas9 complex.

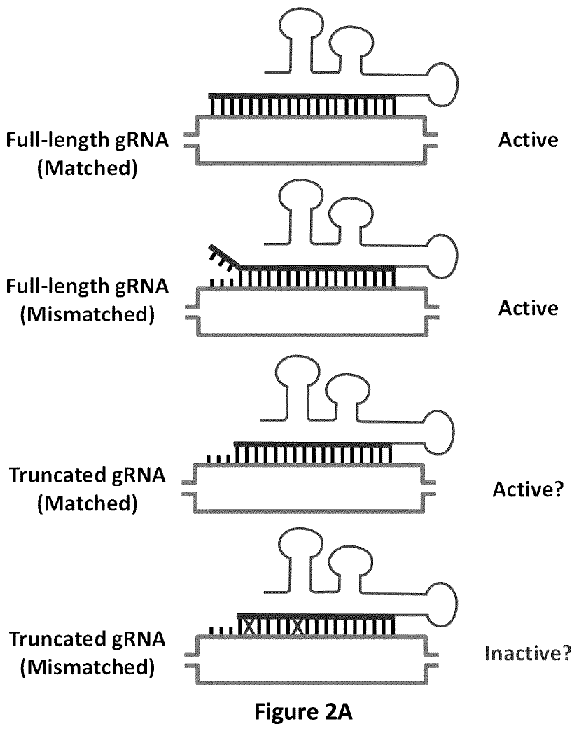

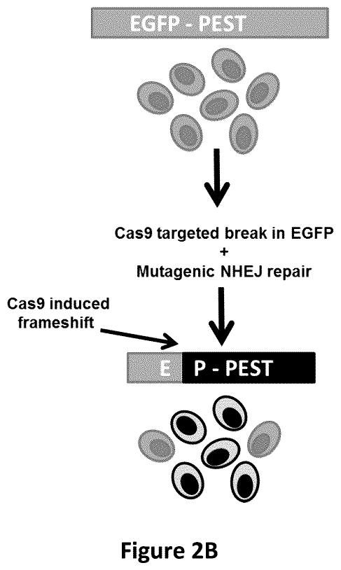

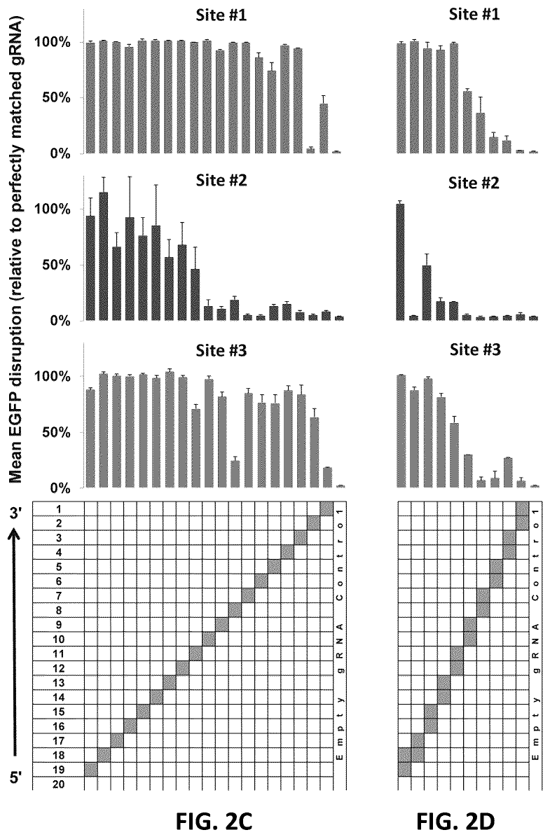

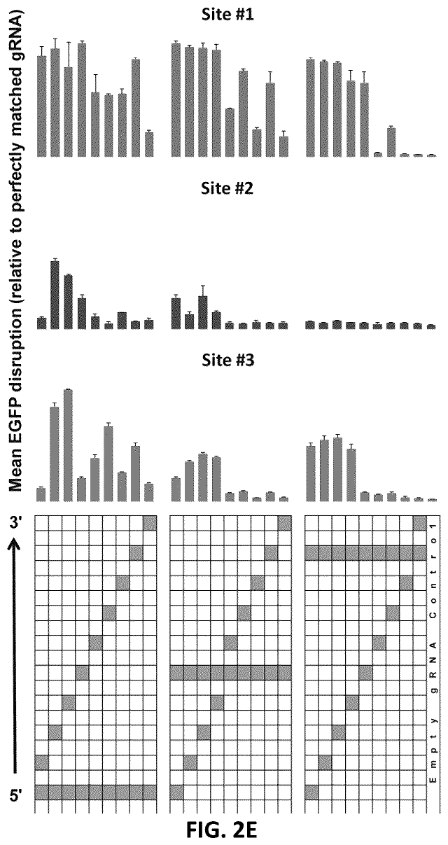

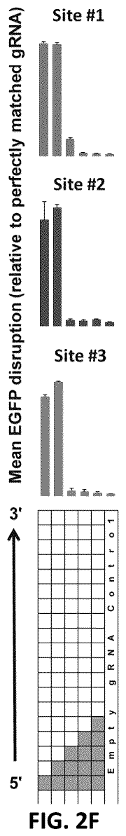

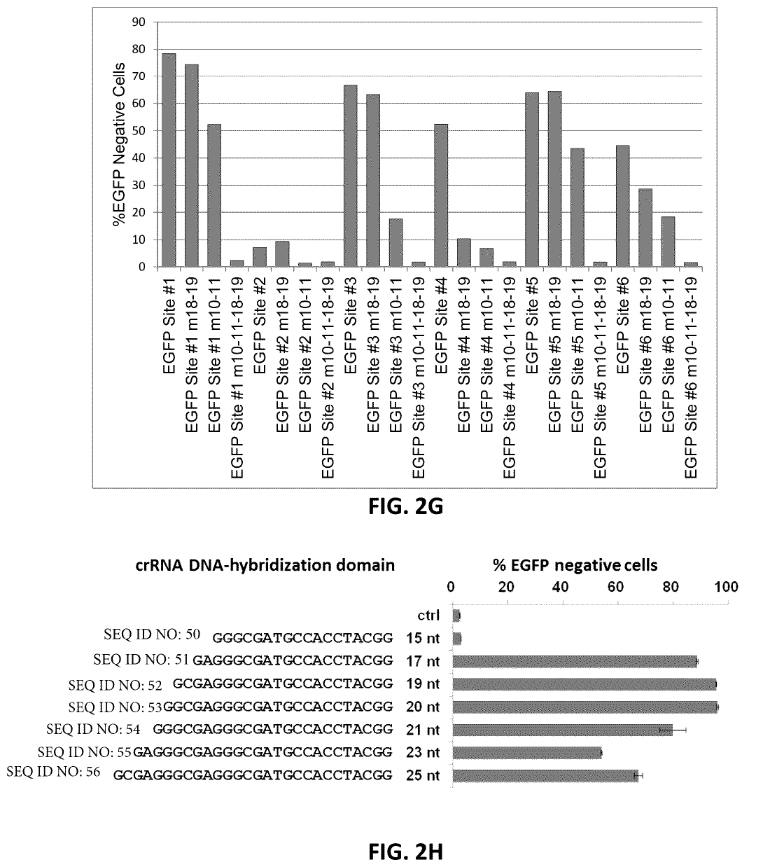

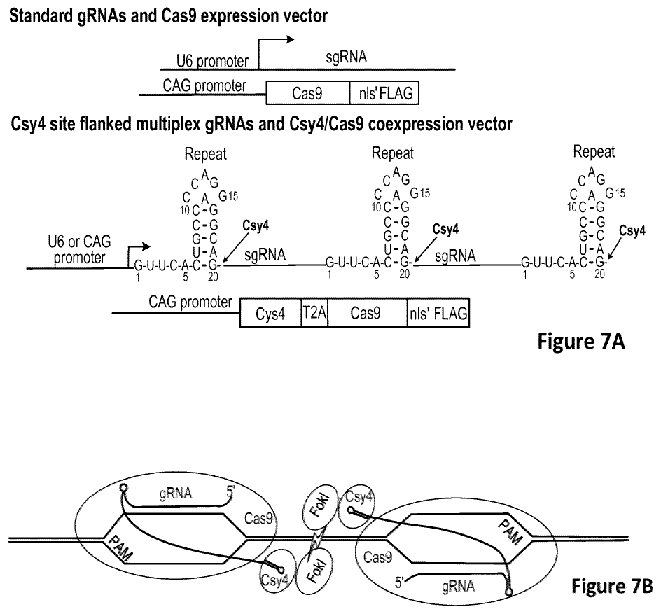

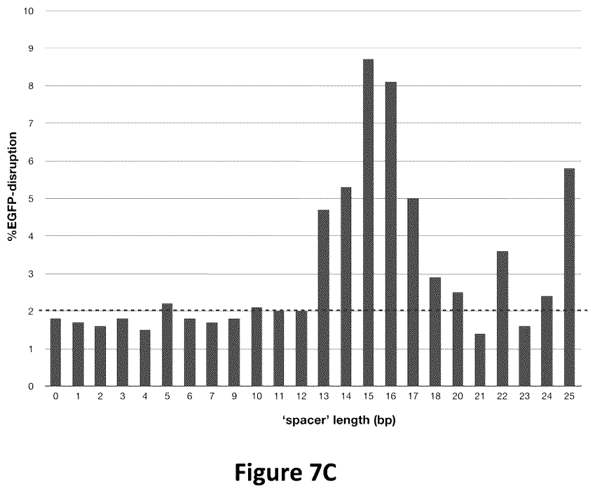

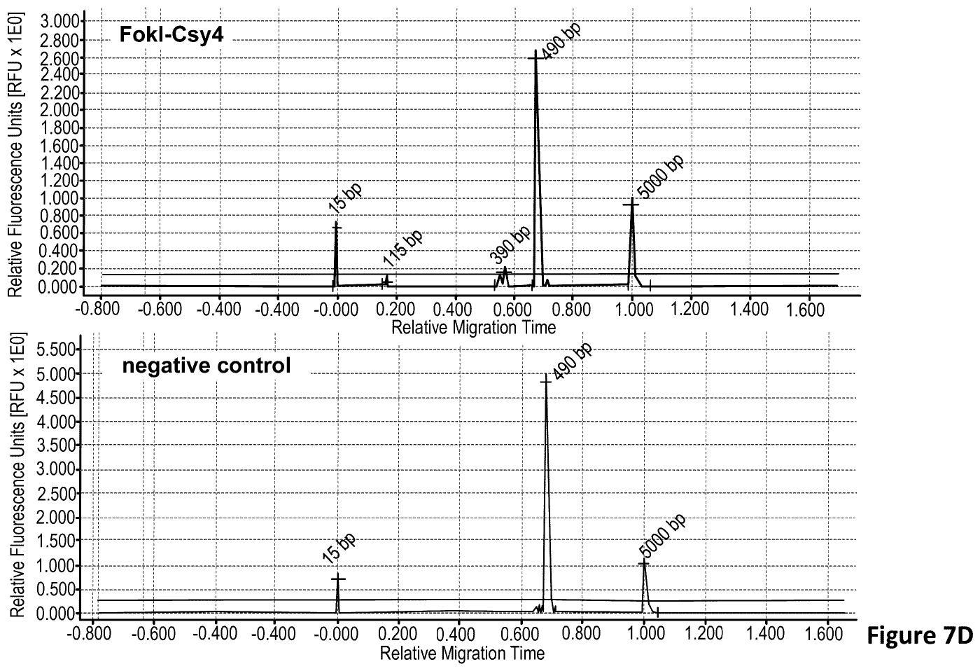

RNA-binding protein/RNA target sequences that could be used would include but are not limited to the lambda N, MS2 or Csy4 proteins. The wild type and high-affinity sequences for MS2 are AAACAUGAGGAUUACCCAUGUCG (SEQ ID NO:19) and AAACAUGAGGAUCACCCAUGUCG (SEQ ID NO:20), respectively (see Keryer-Bibens et al., supra, FIG. 2); the nutL and nutR BoxB sequences to which lambda N binds are GCCCUGAAGAAGGGC (SEQ ID NO:21) and GCCCUGAAAAAGGGC (SEQ ID NO:22), respectively. The sequences to which Csy4 binds are GUUCACUGCCGUAUAGGCAG (SEQ ID NO:23) or GUUCACUGCCGUAUAGGCAGCUAAGAAA (SEQ ID NO:24). The binding sites can be attached to 3′ end of a gRNA sequence and gRNAs harboring this additional Csy4 binding site can still direct Cas9 to cleave specific sequences in human cells and thus remain functional in the cell (Example 2 and FIG. 7).

Thus described herein are three-part fusion guide nucleic acids comprising: (1) a first sequence of 17-20 nts that is complementary to the complementary strand of 17-20 consecutive nucleotides of a target sequence with an adjacent PAM sequence: (2) a second sequence comprising all or part of a Cas9 guide RNA, e.g., all or part of GUUUUAGAGCUAGAAAUAGCAAGUUAAAAUAAGGCUAGUCCGUUAUCA ACUUGAAAAAGUGGCACCGAGTCGGUGCUUUU (SEQ ID NO: 15) or an active portion thereof, UAGCAAGUUAAAAUAAGGCUAGUCCGUUAUCAACUUGAAAAAGUGGCA CCGAGUCGGUGC (SEQ ID NO: 16) or an active portion thereof; or AGCAUAGCAAGUUAAAAUAAGGCUAGUCCGUUAUCAACUUGAAAAAGU GGCACCGAGUCGGUGC (SEQ ID NO:17) or an active portion thereof: and (3) a third sequence that forms a stem-loop structure recognized by an RNA binding protein, e.g., MS2, Csy4, or lambda N. These sequences can be arranged in any order so long as all of the parts retain their function, e.g., (1)-(2)-(3), or (3)-(1)-(2), or (3)-(2)-(1), or (1)-(3)-(2), or (2)-(1)-(3), or (2)-(3)-(1).

In some embodiments wherein (X₁₇₋₂₀)GUUUUAGAGCUAUGCUGUUUUG (SEQ ID NO:6) is used as a crRNA, the following tracrRNA is used: GGAACCAUUCAAAACAGCAUAGCAAGUUAAAAUAAGGCUAGUCCGUUA UCAACUUGAAAAAGUGGCACCGAGUCGGUGC (SEQ ID NO:15) or an active portion thereof. In some embodiments wherein (X₁₇₋₂₀)GUUUUAGAGCUA (SEQ ID NO:5) is used as a crRNA, the following tracrRNA is used: UAGCAAGUUAAAAUAAGGCUAGUCCGUUAUCAACUUGAAAAAGUGGCA CCGAGUCGGUGC (SEQ ID NO: 16) or an active portion thereof. In some embodiments wherein (X₁₇₋₂₀)GUUUUAGAGCUAUGCU (SEQ ID NO:7) is used as a crRNA, the following tracrRNA is used: AGCAUAGCAAGUUAAAAUAAGGCUAGUCCGUUAUCAACUUGAAAAAGU GGCACCGAGUCGGUGC (SEQ ID NO:17) or an active portion thereof.

In some embodiments, there are additional nucleotides, e.g., up to 20 additional nucleotides, that act as a flexible linker between Csy4 and the gRNA: these nucleotides should not add any secondary or tertiary structure to the gRNA. For example the sequence ‘GTTC’ has been shown to be unstructured and could be construed as ‘linker’ sequence.

In some embodiments, the wild-type Csy4 binding sequence is used, which is: GUUCACUGCCGUAUAGGCAGCUAAGAAA (SEQ ID NO:24). In some embodiments, a truncated Csy4 binding sequence is used, which upon processing by Csy4 produces gRNAs of higher activity. This sequence is

(SEQ ID NO: 23)
GUUCACUGCCGUAUAGGCAG.

Also provided are fusion proteins comprising an RNA binding protein, e.g., MS2, Csy4, or lambda N, linked to a catalytic domain of a HFD, e.g., a FokI nuclease as described above, optionally with an intervening linker of 2-30, e.g., 5-20 nts, as well as nucleic acids encoding the same.

### MS2/Lambda N/Csy4

Exemplary sequences for the MS2, lambda N, and Csy4 proteins are given below; the MS2 functions as a dimer, therefore the MS2 protein can include a fused single chain dimer sequence.

1. Exemplary Sequences for Fusions of Single MS2 Coat Protein (Wt, N55K or deltaFG) to the N-Terminus or C-Terminus of FokI.

MS2 coat protein amino acid sequence:
(SEQ ID NO: 25)
MASNFTQFVLVDNGGTGDVTVAPSNFANGVAEWISSNSRSQAYKVTC
SVRQSSAQNRKYTIKVEVPKVATQTVGGVELPVAAWRSYLNMELTIP
IFATNSDCELIVKAMQGLLKDGNPIPSAIAANSGIY
MS2 N55K:
(SEQ ID NO: 26)
MASNFTQFVLVDNGGTGDVTVAPSNFANGVAEWISSNSRSQAYKVTC
SVRQSSAQKRKYTIKVEVPKVATQTVGGVELPVAAWRSYLNMELTIP
IFATNSDCELIVKAMQGLLKDGNPIPSAIAANSGIY
MS2deltaFG:
(SEQ ID NO: 27)
MASNFTQFVLVDNGGTGDVTVAPSNFANGIAEWISSNSRSQAYKVTC
SVRQSSAQNRKYTIKVEVPKGAWRSYLNMELTIPIFATNSDCELIVK
AMQGLLKDGNPIPSAIAANSGIY

2. Exemplary Sequences for Fusions of Fused Dimeric MS2 Coat Protein (Wt, N55K or deltaFG) to the N-Terminus or C-Terminus of FokI.

Dimeric MS2 coat protein:
(SEQ ID NO: 28)
MASNFTQFVLVDNGGTGDVTVAPSNFANGVAEWISSNSRSQAYKVTC
SVRQSSAQNRKYTIKVEVPKVATQTVGGVELPVAAWRSYLNMELTIP
IFATNSDCELIVKAMQGLLKDGNPIPSAIAANSGLYGAMASNFTQFV
LVDNGGTGDVTVAPSNFANGVAEWISSNSRSQAYKVTCSVRQSSAQN
RKYTIKVEVPKVATQTVGGVELPVAAWRSYLNMELTIPIFATNSDCE
LIVKAMQGLLKDGNPIPSAIAANSLIN
Dimeric MS2 N55K:
(SEQ ID NO: 29)
MASNFTQFVLVDNGGTGDVTVAPSNFANGVAEWISSNSRSQAYKVTC
SVRQSSAQKRKYTIKVEVPKVATQTVGGVELPVAAWRSYLNMELTIP
IFATNSDCELIVKAMQGLLKDGNPIPSAIAANSGLYGAMASNFTQFV
LVDNGGTGDVTVAPSNFANGVAEWISSNSRSQAYKVTCSVRQSSAQK
RKYTIKVEVPKVATQTVGGVELPVAAWRSYLNMELTIPIFATNSDCE
LIVKAMQGLLKDGNPIPSAIAANSLIN
Dimeric MS2deltaFG:
(SEQ ID NO: 30)
MASNFTQFVLVDNGGTGDVTVAPSNFANGVAEWISSNSRSQAYKVTC
SVRQSSAQKRKYTIKVEVPKGAWRSYLNMELTIPIFATNSDCELIVK
AMQGLLKDGNPIPSAIAANSGLYGAMASNFTQFVLVDNGGTGDVTVA
PSNFANGVAEWISSNSRSQAYKVTCSVRQSSAQKRKYTIKVEVPKGA
WRSYLNMELTIPIFATNSDCELIVKAMQGLLKDGNPIPSAIAANSLI
N

### 3. Exemplary Sequences for Fusions of Lambda N to N-Terminus or C-Terminus of FokI.

Lambda N amino acid sequence:
(SEQ ID NO: 31)
MDAQTRRRERRAEKQAQWKAAN
or
(SEQ ID NO: 32)
MDAQTRRRERRAEKQAQWKAANPLLVGVSAKPVNRPILSLNRKPKSR
VESALNPIDLTVLAEYHKQIESNLQRIERKNQRTWYSKPGERGITCS
GRQKIKGKSIPLI

### 4. Exemplary Sequence for Fusions of Csy4 to N-Terminus or C-Terminus of Dcas9

Exemplary sequences for Cys4 are given in Haurwitz et al. 329(5997); 1355-8 (2010), e.g., the inactivated form; for example see the Csy4 homologues from Pseudomonas aeruginosa UCBPP-PA14 (Pal4), Yersinia pestis AAM85295 (Yp), Escherichia coli UT189 (Ec89), Dichelobacter nodosus VCS1703A (Dn), Acinetobacter baumannii AB0057 (Ab), Moritella sp. PE36 (MP1, MP01), Shewanella sp. W3-18-1 (SW), Pasteurella multocida subsp. multocida Pm70 (Pm), Pectobacterium wasabiae (Pw), and Dickeya dadantii Ech703 (Dd) that are set forth in FIG. S6 of Haurwitz et al., Science 329(5997); 1355-1358 (2010). In preferred embodiments, the Csy4 is from Pseudomonas aeruginosa.

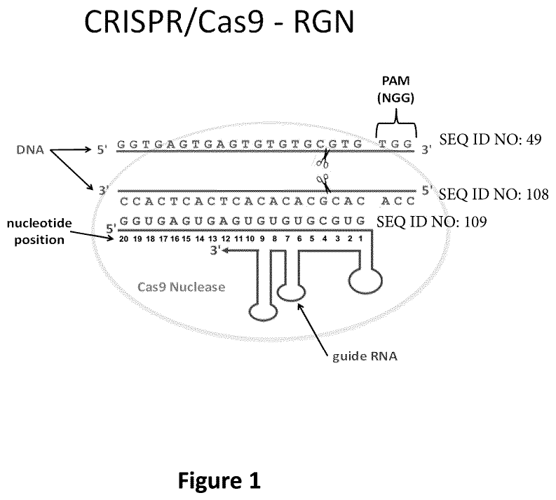

Methods of using the fusions include contacting a cell with or expressing in a cell a pair of three-part fusion guide nucleic acids that include sequences complementary to a single region of a target DNA, a RNA-binding protein linked to a catalytic domain of a FokI nuclease, and a Cas9 protein (e.g., the inactive dCas9 protein from S. pyogenes, either as encoded in bacteria or codon-optimized for expression in mammalian cells, containing mutations at D10, E762, H983, D986, H840, or N863, e.g., D10A/D1ON and H840A/H840N/H840Y, to render the nuclease portion of the protein catalytically inactive; substitutions at these positions could be alanine (as they are in Nishimasu al., Cell 156, 935-949 (2014)) or they could be other residues, e.g., glutamine, asparagine, tyrosine, serine, or aspartate, e.g., E762Q, H983N, H983Y, D986N, N863D, N863S, or N863H (FIG. 1C).). The two gRNA target sites may be overlapping or some distance away from each other, e.g., up to about 200 nts apart, e.g., less than 100, 50, 25, 10, 5, 4, or 2 nts apart.

### FokI

FokI is a type IIs restriction endonuclease that includes a DNA recognition domain and a catalytic (endonuclease) domain. The fusion proteins described herein can include all of FokI or just the catalytic endonuclease domain, i.e., amino acids 388-583 or 408-583 of GenBank Acc. No. AAA24927.1, e.g., as described in Li et al., Nucleic Acids Res. 39(1); 359-372 (2011); Cathomen and Joung, Mol. Ther. 16: 1200-1207 (2008), or a mutated form of FokI as described in Miller et al. Nat Biotechnol 25: 778-785 (2007); Szczepek et al., Nat Biotechnol 25: 786-793 (2007); or Bitinaite et al., Proc. Natl. Acad. Sci. USA. 95:10570-10575 (1998).

An exemplary amino acid sequence of FokI is as follows:

```
(SEQ ID NO: 33)
                10 20 30 40
MFLSMVSKIR TFGWVQNPGK FENLKRVVQV FDRNSKVHNE
                50 60 70 80
VKNIKIPTLV KESKIQKELV AIMNQHDLIY TYKELVGTGT
                90 100 110 120
SIRSEAPCDA IIQATIADQG NKKGYIDNWS SDGFLRWAHA
              130 140 150 160
LGFIEYINKS DSFVITDVGL AYSKSADGSA IEKEILIEAI
              170 180 190 200
SSYPPAIRIL TLLEDGQHLT KFDLGKNLGF SGESGFTSLP
              210 220 230 240
EGILLDTLAN AMPKDKGEIR NNWEGSSDKY ARMIGGWLDK
              250 260 270 280
LGLVKQGKKE FIIPTLGKPD NKEFISHAFK ITGEGLKVLR
              290 300 310 320
RAKGSTKFTR VPKRVYWEML ATNLTDKEYV RTRRALILEI
              330 340 350 360
LIKAGSLKIE QIQDNLKKLG FDEVIETIEN DIKGLINTGI
              370 380 390 400
FIEIKGRFYQ LKDHILQFVI PNRGVTKQLV KSELEEKKSE
              410 420 430 440
LRHKLKYVPH EYIELIEIAR NSTQDRILEM KVMEFFMKVY
              450 460 470 480
GYRGKHLGGS RKPDGAIYTV GSPIDYGVIV DTKAYSGGYN
              490 500 510 520
LPIGQADEMQ RYVEENQTRN KHINPNEWWK VYPSSVTEFK
              530 540 550 560
FLFVSGHFKG NYKAQLTRLN HITNCNGAVL SVEELLIGGE
              570 580
MIKAGTLTLE EVRRKFNNGE INF
```

An exemplary nucleic acid sequence encoding FokI is as follows:

(SEQ ID NO: 34)
ATGTTTTTGAGTATGGTTTCTAAAATAAGAACTTTCGGTTGGGTTCAA
AATCCAGGTAAATTTGAGAATTTAAAACGAGTAGTTCAAGTATTTGAT
AGAAATTCTAAAGTACATAATGAAGTGAAAAATATAAAGATACCAACC
CTAGTCAAAGAAAGTAAGATCCAAAAAGAACTAGTTGCTATTATGAAT
CAACATGATTTGATTTATACATATAAAGAGTTAGTAGGAACAGGAACT
TCAATACGTTCAGAAGCACCATGCGATGCAATTATTCAAGCAACAATA
GCAGATCAAGGAAATAAAAAAGGCTATATCGATAATTGGTCATCTGAC
GGTTTTTTGCGTTGGGCACATGCTTTAGGATTTATTGAATATATAAAT
AAAAGTGATTCTTTTGTAATAACTGATGTTGGACTTGCTTACTCTAAA
TCAGCTGACGGCAGCGCCATTGAAAAAGAGATTTTGATTGAAGCGATA
TCATCTTATCCTCCAGCGATTCGTATTTTAACTTTGCTAGAAGATGGA
CAACATTTGACAAAGTTTGATCTTGGCAAGAATTTAGGTTTTAGTGGA
GAAAGTGGATTTACTTCTCTACCGGAAGGAATTCTTTTAGATACTCTA
GCTAATGCTATGCCTAAAGATAAAGGCGAAATTCGTAATAATTGGGAA
GGATCTTCAGATAAGTACGCAAGAATGATAGGTGGTTGGCTGGATAAA
CTAGGATTAGTAAAGCAAGGAAAAAAAGAATTTATCATTCCTACTTTG
GGTAAGCCGGACAATAAAGAGTTTATATCCCACGCTTTTAAAATTACT
GGAGAAGGTTTGAAAGTACTGCGTCGAGCAAAAGGCTCTACAAAATTT
ACACGTGTACCTAAAAGAGTATATTGGGAAATGCTTGCTACAAACCTA
ACCGATAAAGAGTATGTAAGAACAAGAAGAGCTTTGATTTTAGAAATA
TTAATCAAAGCTGGATCATTAAAAATAGAACAAATACAAGACAACTTG
AAGAAATTAGGATTTGATGAAGTTATAGAAACTATTGAAAATGATATC
AAAGGCTTAATTAACACAGGTATATTTATAGAAATCAAAGGGCGATTT
TATCAATTGAAAGACCATATTCTTCAATTTGTAATACCTAATCGTGGT
GTGACTAAGCAACTAGTCAAAAGTGAACTGGAGGAGAAGAAATCTGAA
CTTCGTCATAAATTGAAATATGTGCCTCATGAATATATTGAATTAATT
GAAATTGCCAGAAATTCCACTCAGGATAGAATTCTTGAAATGAAGGTA
ATGGAATTTTTTATGAAAGTTTATGGATATAGAGGTAAACATTTGGGT
GGATCAAGGAAACCGGACGGAGCAATTTATACTGTCGGATCTCCTATT
GATTACGGTGTGATCGTGGATACTAAAGCTTATAGCGGAGGTTATAAT
CTGCCAATTGGCCAAGCAGATGAAATGCAACGATATGTCGAAGAAAAT
CAAACACGAAACAAACATATCAACCCTAATGAATGGTGGAAAGTCTAT
CCATCTTCTGTAACGGAATTTAAGTTTTTATTTGTGAGTGGTCACTTT
AAAGGAAACTACAAAGCTCAGCTTACACGATTAAATCATATCACTAAT
TGTAATGGAGCTGTTCTTAGTGTAGAAGAGCTTTTAATTGGTGGAGAA
ATGATTAAAGCCGGCACATTAACCTTAGAGGAAGTGAGACGGAAATTT
AATAACGGCGAGATAAACTTTTAA

In some embodiments, the FokI nuclease used herein is at least about 50% identical SEQ ID NO:33, e.g., to amino acids 388-583 or 408-583 of SEQ ID NO:33. These variant nucleases must retain the ability to cleave DNA. In some embodiments, the nucleotide sequences are about 50%, 55%, 60%, 65%, 70%, 75%, 80%, 85%, 90%, 95%, 99% or 100% identical to amino acids 388-583 or 408-583 of SEQ ID NO:4. In some embodiments, any differences from amino acids 388-583 or 408-583 of SEQ ID NO:4 are in non-conserved regions.

To determine the percent identity of two sequences, the sequences are aligned for optimal comparison purposes (gaps are introduced in one or both of a first and a second amino acid or nucleic acid sequence as required for optimal alignment, and non-homologous sequences can be disregarded for comparison purposes). The length of a reference sequence aligned for comparison purposes is at least 50% (in some embodiments, about 50%, 55%, 60%, 65%, 70%, 75%, 85%, 90%, 95%, or 100% of the length of the reference sequence is aligned). The nucleotides or residues at corresponding positions are then compared. When a position in the first sequence is occupied by the same nucleotide or residue as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For purposes of the present application, the percent identity between two amino acid sequences is determined using the Needleman and Wunsch ((1970) J. Mol. Biol. 48:444-453) algorithm which has been incorporated into the GAP program in the GCG software package, using a Blossum 62 scoring matrix with a gap penalty of 12, a gap extend penalty of 4, and a frameshift gap penalty of 5.

### Heterologous Functional Domains

The transcriptional activation domains can be fused on the N or C terminus of the Cas9. In addition, although the present description exemplifies transcriptional activation domains, other heterologous functional domains (e.g., transcriptional repressors (e.g., KRAB, ERD, SID, and others, e.g., amino acids 473-530 of the ets 2 repressor factor (ERF) repressor domain (ERD), amino acids 1-97 of the KRAB domain of KOX1, or amino acids 1-36 of the Mad mSIN3 interaction domain (SID); see Beerli et al., PNAS USA 95:14628-14633 (1998)) or silencers such as Heterochromatin Protein 1 (HP1, also known as swi6), e.g., HPla or HPIB: proteins or peptides that could recruit long non-coding RNAs (lncRNAs) fused to a fixed RNA binding sequence such as those bound by the MS2 coat protein, endoribonuclease Csy4, or the lambda N protein: enzymes that modify the methylation state of DNA (e.g., DNA methyltransferase (DNMT) or TET proteins); or enzymes that modify histone subunits (e.g., histone acetyltransferases (HAT), histone deacetylases (HDAC), histone methyltransferases (e.g., for methylation of lysine or arginine residues) or histone demethylases (e.g., for demethylation of lysine or arginine residues)) as are known in the art can also be used. A number of sequences for such domains are known in the art, e.g., a domain that catalyzes hydroxylation of methylated cytosines in DNA. Exemplary proteins include the Ten-Eleven-Translocation (TET)1-3 family, enzymes that converts 5-methylcytosine (5-mC) to 5-hydroxymethylcytosine (5-hmC) in DNA.

Sequences for human TET1-3 are known in the art and are shown in the following table:

| | GenBank Accession Nos. | |
|---|---|---|
| Gene | Amino Acid | Nucleic Acid |
| TET1 | NP_085128.2 | NM_030625.2 |
| TET2* | NP_001120680.1 (var 1) | NM_001127208.2 |
| | NP_060098.3 (var 2) | NM_017628.4 |
| TET3 | NP_659430.1 | NM_144993.1 |

*Variant (1) represents the longer transcript and encodes the longer isoform (a). Variant (2) differs in the 5′ UTR and in the 3′ UTR and coding sequence compared to variant 1. The resulting isoform (b) is shorter and has a distinct C-terminus compared to isoform a.

In some embodiments, all or part of the full-length sequence of the catalytic domain can be included, e.g., a catalytic module comprising the cysteine-rich extension and the 20GFeDO domain encoded by 7 highly conserved exons, e.g., the Tet1 catalytic domain comprising amino acids 1580-2052, Tet2 comprising amino acids 1290-1905 and Tet3 comprising amino acids 966-1678. See, e.g., FIG. 1 of Iyer et al., Cell Cycle. 2009 Jun 1:8(11); 1698-710. Epub 2009 Jun 27, for an alignment illustrating the key catalytic residues in all three Tet proteins, and the supplementary materials thereof (available at ftp site ftp.ncbi.nih.gov/pub/aravind/DONS/supplementary_material_DONS.html) for full length sequences (see, e.g., seq 2c); in some embodiments, the sequence includes amino acids 1418-2136 of Tet1 or the corresponding region in Tet2/3.

Other catalytic modules can be from the proteins identified in Iyer et al., 2009.

In some embodiments, the heterologous functional domain is a biological tether, and comprises all or part of (e.g., DNA binding domain from) the MS2 coat protein, endoribonuclease Csy4, or the lambda N protein. These proteins can be used to recruit RNA molecules containing a specific stem-loop structure to a locale specified by the dCas9 gRNA targeting sequences. For example, a dCas9 fused to MS2 coat protein, endoribonuclease Csy4, or lambda N can be used to recruit a long non-coding RNA (lncRNA) such as XIST or HOTAIR: see, e.g., Keryer-Bibens et al., Biol. Cell 100:125-138 (2008), that is linked to the Csy4, MS2 or lambda N binding sequence. Alternatively, the Csy4, MS2 or lambda N protein binding sequence can be linked to another protein, e.g., as described in Keryer-Bibens et al., supra, and the protein can be targeted to the dCas9 binding site using the methods and compositions described herein. In some embodiments, the Csy4 is catalytically inactive.

In some embodiments, the fusion proteins include a linker between the dCas9 and the heterologous functional domains. Linkers that can be used in these fusion proteins (or between fusion proteins in a concatenated structure) can include any sequence that does not interfere with the function of the fusion proteins. In preferred embodiments, the linkers are short, e.g., 2-20 amino acids, and are typically flexible (i.e., comprising amino acids with a high degree of freedom such as glycine, alanine, and serine). In some embodiments, the linker comprises one or more units consisting of GGGS (SEQ ID NO:14) or GGGGS (SEQ ID NO:15), e.g., two, three, four, or more repeats of the GGGS (SEQ ID NO:14) or GGGGS (SEQ ID NO:15) unit. Other linker sequences can also be used.

### Cas9

Cas9 molecules of a variety of species can be used in the methods and compositions described herein. While the S. pyogenes and S. thermophilus Cas9 molecules are the subject of much of the disclosure herein, Cas9 molecules of, derived from, or based on the Cas9 proteins of other species listed herein can be used as well. In other words, while the much of the description herein uses S. pyogenes and S. thermophilus Cas9 molecules, Cas9 molecules from the other species can replace them. Such species include those set forth in the following table, which was created based on supplementary FIG. 1 of Chylinski et al., 2013.

Alternative Cas9 proteins

| GenBank | |
|---|---|
| Acc No. | Bacterium |
| 303229466 | Veillonella atypica ACS-134-V-Col7a |
| 34762592 | Fusobacterium nucleatum subsp. vincentii |
| 374307738 | Filifactor alocis ATCC 35896 |
| 320528778 | Solobacterium moorei F0204 |
| 291520705 | Coprococcus catus GD-7 |
| 42525843 | Treponema denticola ATCC 35405 |
| 304438954 | Peptoniphilus duerdenii ATCC BAA-1640 |
| 224543312 | Catenibacterium mitsuokai DSM 15897 |
| 24379809 | Streptococcus mutans UA159 |
| 15675041 | Streptococcus pyogenes SF370 |
| 16801805 | Listeria innocua Clip11262 |
| 116628213 | Streptococcus thermophilus LMD-9 |
| 323463801 | Staphylococcus pseudintermedius ED99 |
| 352684361 | Acidaminococcus intestini RyC-MR95 |
| 302336020 | Olsenella uli DSM 7084 |
| 366983953 | Oenococcus kitaharae DSM 17330 |
| 310286728 | Bifidobacterium bifidum S17 |
| 258509199 | Lactobacillus rhamnosus GG |
| 300361537 | Lactobacillus gasseri JV-V03 |
| 169823755 | Finegoldia magna ATCC 29328 |
| 47458868 | Mycoplasma mobile 163K |
| 284931710 | Mycoplasma gallisepticum str. F |
| 363542550 | Mycoplasma ovipneumoniae SC01 |
| 384393286 | Mycoplasma canis PG 14 |
| 71894592 | Mycoplasma synoviae 53 |
| 238924075 | Eubacterium rectale ATCC 33656 |
| 116627542 | Streptococcus thermophilus LMD-9 |
| 315149830 | Enterococcus faecalis TX0012 |
| 315659848 | Staphylococcus lugdunensis M23590 |
| 160915782 | Eubacterium dolichum DSM 3991 |
| 336393381 | Lactobacillus coryniformis subsp. torquens |
| 310780384 | Ilyobacter polytropus DSM 2926 |
| 325677756 | Ruminococcus albus 8 |
| 187736489 | Akkermansia muciniphila ATCC BAA-835 |
| 117929158 | Acidothermus cellulolyticus 11B |
| 189440764 | Bifidobacterium longum DJO10A |
| 283456135 | Bifidobacterium dentium Bd1 |
| 38232678 | Corynebacterium diphtheriae NCTC 13129 |
| 187250660 | Elusimicrobium minutum Pei191 |
| 319957206 | Nitratifractor salsuginis DSM 16511 |
| 325972003 | Sphaerochaeta globus str. Buddy |
| 261414553 | Fibrobacter succinogenes subsp. succinogenes |
| 60683389 | Bacteroides fragilis NCTC 9343 |
| 256819408 | Capnocytophaga ochracea DSM 7271 |
| 90425961 | Rhodopseudomonas palustris BisB18 |
| 373501184 | Prevotella micans F0438 |
| 294674019 | Prevotella ruminicola 23 |
| 365959402 | Flavobacterium columnare ATCC 49512 |
| 312879015 | Aminomonas paucivorans DSM 12260 |
| 83591793 | Rhodospirillum rubrum ATCC 11170 |
| 294086111 | Candidatus Puniceispirillum marinum IMCC1322 |
| 121608211 | Verminephrobacter eiseniae EF01-2 |
| 344171927 | Ralstonia syzygii R24 |
| 159042956 | Dinoroseobacter shibae DFL 12 |
| 288957741 | Azospirillum sp-B510 |
| 92109262 | Nitrobacter hamburgensis X14 |
| 148255343 | Bradyrhizobium sp-BTAi1 |
| 34557790 | Wolinella succinogenes DSM 1740 |
| 218563121 | Campylobacter jejuni subsp. jejuni |
| 291276265 | Helicobacter mustelae 12198 |
| 229113166 | Bacillus cereus Rock1-15 |
| 222109285 | Acidovorax ebreus TPSY |
| 189485225 | uncultured Termite group 1 |
| 182624245 | Clostridium perfringens D str. |
| 220930482 | Clostridium cellulolyticum H10 |
| 154250555 | Parvibaculum lavamentivorans DS-1 |
| 257413184 | Roseburia intestinalis L1-82 |
| 218767588 | Neisseria meningitidis Z2491 |
| 15602992 | Pasteurella multocida subsp. multocida |
| 319941583 | Sutterella wadsworthensis 3 1 |
| 254447899 | gamma proteobacterium HTCC5015 |
| 54296138 | Legionella pneumophila str. Paris |
| 331001027 | Parasutterella excrementihominis YIT 11859 |
| 34557932 | Wolinella succinogenes DSM 1740 |
| 118497352 | Francisella novicida U112 |

The constructs and methods described herein can include the use of any of those Cas9 proteins, and their corresponding guide RNAs or other guide RNAs that are compatible. The Cas9 from Streptococcus thermophilus LMD-9 CRISPR1 system has also been shown to function in human cells in Cong et al (Science 339, 819 (2013)). Cas9 orthologs from N. meningitides are described in Hou et al., Proc Natl Acad Sci USA. 2013 Sep. 24; 110(39);15644-9 and Esvelt et al., Nat Methods. 2013 Nov;10(11);1116-21. Additionally, Jinek et al. showed in vitro that Cas9 orthologs from S. thermophilus and L. innocua, (but not from N. meningitidis or C. jejuni, which likely use a different guide RNA), can be guided by a dual S. pyogenes gRNA to cleave target plasmid DNA, albeit with slightly decreased efficiency.

In some embodiments, the present system utilizes the Cas9 protein from S. pyogenes, either as encoded in bacteria or codon-optimized for expression in mammalian cells. In some embodiments, a catalytically inactive Cas9 (dCas9) containing mutations at (i) D10, E762, H983, or D986 and (i) H840 or N863, e.g., D10A/D10N and H840A/H840N/H840Y, to render the nuclease portion of the protein completely catalytically inactive; substitutions at these positions could be alanine (as they are in Nishimasu al., Cell 156, 935-949 (2014)) or they could be other residues, e.g., glutamine, asparagine, tyrosine, serine, or aspartate, e.g., E762Q, H983N, H983Y, D986N, N863D, N863S, or N863H (FIG. 1C). To render the Cas9 partially inactive, e.g., to create a nickase that cuts only one strand, a mutation at any of D10, E762, H983, D986, H840, or N863 can be introduced. The wild type sequence of S. pyogenes Cas9 nuclease that can be used in the methods and compositions described herein is as follows.

```
(SEQ ID NO: 18)
                10 20 30 40
MDKKYSIGLD IGTNSVGWAV ITDEYKVPSK KFKVLGNTDR
                50 60 70 80
HSIKKNLIGA LLFDSGETAE ATRLKRTARR RYTRRKNRIC
                90 100 110 120
YLQEIFSNEM AKVDDSFFHR LEESFLVEED KKHERHPIFG
              130 140 150 160
NIVDEVAYHE KYPTIYHLRK KLVDSTDKAD LRLIYLALAH
              170 180 190 200
MIKFRGHFLI EGDLNPDNSD VDKLFIQLVQ TYNQLFEENP
              210 220 230 240
INASGVDAKA ILSARLSKSR RLENLIAQLP GEKKNGLFGN
              250 260 270 280
LIALSLGLTP NFKSNFDLAE DAKLQLSKDT YDDDLDNLLA
              290 300 310 320
QIGDQYADLF LAAKNLSDAI LLSDILRVNT EITKAPLSAS
              330 340 350 360
MIKRYDEHHQ DLTLLKALVR QQLPEKYKEI FFDQSKNGYA
              370 380 390 400
GYIDGGASQE EFYKFIKPIL EKMDGTEELL VKLNREDLLR
              410 420 430 440
KQRTFDNGSI PHQIHLGELH AILRRQEDFY PFLKDNREKI
              450 460 470 480
EKILTFRIPY YVGPLARGNS RFAWMTRKSE ETITPWNFEE
              490 500 510 520
VVDKGASAQS FIERMTNFDK NLPNEKVLPK HSLLYEYFTV
              530 540 550 560
YNELTKVKYV TEGMRKPAFL SGEQKKAIVD LLFKTNRKVT
              570 580 590 600
VKQLKEDYFK KIECFDSVEI SGVEDRFNAS LGTYHDLLKI
              610 620 630 640
IKDKDFLDNE ENEDILEDIV LTLTLFEDRE MIEERLKTYA
              650 660 670 680
HLFDDKVMKQ LKRRRYTGWG RLSRKLINGI RDKQSGKTIL
              690 700 710 720
DFLKSDGFAN RNFMQLIHDD SLTFKEDIQK AQVSGQGDSL
              730 740 750 760
HEHIANLAGS PAIKKGILQT VKVVDELVKV MGRHKPENIV
              770 780 790 800
IEMARENQTT QKGQKNSRER MKRIEEGIKE LGSQILKEHP
              810 820 830 840
VENTQLQNEK LYLYYLQNGR DMYVDQELDI NRLSDYDVDH
              850 860 870 880
IVPQSFLKDD SIDNKVLTRS DKNRGKSDNV PSEEVVKKMK
              890 900 910 920
NYWRQLLNAK LITQRKFDNL TKAERGGLSE LDKAGFIKRQ
              930 940 950 960
LVETRQITKH VAQILDSRMN TKYDENDKLI REVKVITLKS
              970 980 990 1000
KLVSDFRKDF QFYKVREINN YHHAHDAYLN AVVGTALIKK
            1010 1020 1030 1040
YPKLESEFVY GDYKVYDVRK MIAKSEQEIG KATAKYFFYS
            1050 1060 1070 1080
NIMNFFKTEI TLANGEIRKR PLIETNGETG EIVWDKGRDF
            1090 1100 1110 1120
ATVRKVLSMP QVNIVKKTEV QTGGFSKESI LPKRNSDKLI
            1130 1140 1150 1160
ARKKDWDPKK YGGFDSPTVA YSVLVVAKVE KGKSKKLKSV
            1170 1180 1190 1200
KELLGITIME RSSFEKNPID FLEAKGYKEV KKDLIIKLPK
            1210 1220 1230 1240
YSLFELENGR KRMLASAGEL QKGNELALPS KYVNFLYLAS
            1250 1260 1270 1280
HYEKLKGSPE DNEQKQLFVE QHKHYLDEII EQISEFSKRV
            1290 1300 1310 1320
ILADANLDKV LSAYNKHRDK PIREQAENII HLFTLTNLGA
            1330 1340 1350 1360
PAAFKYFDTT IDRKRYTSTK EVLDATLIHQ SITGLYETRI
DLSQLGGD
```

In some embodiments, the Cas9 nuclease used herein is at least about 50% identical to the sequence of S. pyogenes Cas9, i.e., at least 50% identical to SEQ ID NO:18. In some embodiments, the nucleotide sequences are about 50%, 55%, 60%, 65%, 70%, 75%, 80%, 85%, 90%, 95%, 99% or 100% identical to SEQ ID NO:18. In some embodiments, any differences from SEQ ID NO: 18 are in non-conserved regions, as identified by sequence alignment of sequences set forth in Chylinski et al., RNA Biology 10:5, 1-12; 2013 (e.g., in supplementary FIG. 1 and supplementary table 1 thereof); Esvelt et al., Nat Methods. 2013 November; 10(11);1116-21 and Fonfara et al., Nucl. Acids Res. (2014) 42 (4); 2577-2590. [Epub ahead of print 2013 Nov 22] doi: 10.1093/nar/gkt1074.

To determine the percent identity of two sequences, the sequences are aligned for optimal comparison purposes (gaps are introduced in one or both of a first and a second amino acid or nucleic acid sequence as required for optimal alignment, and non-homologous sequences can be disregarded for comparison purposes). The length of a reference sequence aligned for comparison purposes is at least 50% (in some embodiments, about 50%, 55%, 60%, 65%, 70%, 75%, 85%, 90%, 95%, or 100% of the length of the reference sequence is aligned). The nucleotides or residues at corresponding positions are then compared. When a position in the first sequence is occupied by the same nucleotide or residue as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For purposes of the present application, the percent identity between two amino acid sequences is determined using the Needleman and Wunsch ((1970) J. Mol. Biol. 48:444-453) algorithm which has been incorporated into the GAP program in the GCG software package, using a Blossum 62 scoring matrix with a gap penalty of 12, a gap extend penalty of 4, and a frameshift gap penalty of 5.

### Expression Systems

In order to use the fusion proteins and guide RNAs described, it may be desirable to express the engineered proteins from a nucleic acid that encodes them. This can be performed in a variety of ways. For example, the nucleic acid encoding the fusion protein or guide RNA can be cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the fusion protein or guide RNA for production of the fusion protein or guide RNA. The nucleic acid encoding the fusion protein or guide RNA can also be cloned into an expression vector, for administration to a plant cell, animal cell, preferably a mammalian cell or a human cell, fungal cell, bacterial cell, or protozoan cell.

To obtain expression, a sequence encoding a fusion protein or guide RNA is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available.

The promoter used to direct expression of a fusion protein nucleic acid depends on the particular application. For example, a strong constitutive promoter is typically used for expression and purification of fusion proteins. In contrast, when the fusion protein is to be administered in vivo for gene regulation, either a constitutive or an inducible promoter can be used, depending on the particular use of the fusion protein. In addition, a preferred promoter for administration of the fusion protein can be a weak promoter, such as HSV TK or a promoter having similar activity. The promoter can also include elements that are responsive to transactivation, e.g., hypoxia response elements, Gal4 response elements, lac repressor response element, and small molecule control systems such as tetracycline-regulated systems and the RU-486 system (see, e.g., Gossen & Bujard, 1992, Proc. Natl. Acad. Sci. USA, 89:5547: Oligino et al., 1998, Gene Ther., 5:491-496: Wang et al., 1997, Gene Ther., 4:432-441: Neering et al., 1996, Blood, 88: 1147-55; and Rendahl et al., 1998, Nat. Biotechnol., 16:757-761).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the fusion protein, and any signals required, e.g., for efficient polyadenylation of the transcript, transcriptional termination, ribosome binding sites, or translation termination. Additional elements of the cassette may include, e.g., enhancers, and heterologous spliced intronic signals.

The particular expression vector used to transport the genetic information into the cell is selected with regard to the intended use of the fusion protein, e.g., expression in plants, animals, bacteria, fungus, protozoa, etc. Standard bacterial expression vectors include plasmids such as pBR322 based plasmids, pSKF, pET23D, and commercially available tag-fusion expression systems such as GST and LacZ. A preferred tag-fusion protein is the maltose binding protein (MBP). Such tag-fusion proteins can be used for purification of the engineered TALE repeat protein. Epitope tags can also be added to recombinant proteins to provide convenient methods of isolation, for monitoring expression, and for monitoring cellular and subcellular localization, e.g., c-myc or FLAG

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

The vectors for expressing the guide RNAs can include RNA Pol III promoters to drive expression of the guide RNAs, e.g., the H1, U6 or 7SK promoters. These human promoters allow for expression of gRNAs in mammalian cells following plasmid transfection. Alternatively, a T7 promoter may be used, e.g., for in vitro transcription, and the RNA can be transcribed in vitro and purified. Vectors suitable for the expression of short RNAs, e.g., siRNAs, shRNAs, or other small RNAs, can be used.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with the fusion protein encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., 1989, J. Biol. Chem., 264:17619-22: Guide to Protein Purification, in Methods in Enzymology, vol. 182 (Deutscher, ed., 1990)). Transformation of eukaryotic and prokaryotic cells are performed according to standard techniques (see, e.g., Morrison, 1977, J. Bacteriol. 132:349-351: Clark-Curtiss & Curtiss, Methods in Enzymology 101:347-362 (Wu et al., eds, 1983).

Any of the known procedures for introducing foreign nucleotide sequences into host cells may be used. These include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein of choice.

In some embodiments, the fusion protein includes a nuclear localization domain which provides for the protein to be translocated to the nucleus. Several nuclear localization sequences (NLS) are known, and any suitable NLS can be used. For example, many NLSs have a plurality of basic amino acids, referred to as a bipartite basic repeats (reviewed in Garcia-Bustos et al, 1991, Biochim. Biophys. Acta, 1071:83-101). An NLS containing bipartite basic repeats can be placed in any portion of chimeric protein and results in the chimeric protein being localized inside the nucleus. In preferred embodiments a nuclear localization domain is incorporated into the final fusion protein, as the ultimate functions of the fusion proteins described herein will typically require the proteins to be localized in the nucleus. However, it may not be necessary to add a separate nuclear localization domain in cases where the DBD domain itself, or another functional domain within the final chimeric protein, has intrinsic nuclear translocation function.

The present invention includes the vectors and cells comprising the vectors.

### EXAMPLES

The invention is further described in the following examples, which do not limit the scope of the invention described in the claims.

### Example 1. Assessing Specificity of RNA-Guided Endonucleases

CRISPR RNA-guided nucleases (RGNs) have rapidly emerged as a platform for genome editing. This example describes the use of a human cell-based reporter assay to characterize off-target cleavage of CasAS9-based RGNs.

### Materials and Methods

The following materials and methods were used in Example 1.

### Construction of Guide RNAs

DNA oligonucleotides harboring variable 20 nt sequences for Cas9 targeting were annealed to generate short double-strand DNA fragments with 4 bp overhangs compatible with ligation into BsmBI-digested plasmid pMLM3636. Cloning of these annealed oligonucleotides generates plasmids encoding a chimeric+103 single-chain guide RNA with 20 variable 5′ nucleotides under expression of a U6 promoter (Hwang et al., Nat Biotechnol 31, 227-229 (2013); Mali et al., Science 339, 823-826 (2013).). pMLM3636 and the expression plasmid pJDS246 (encoding a codon optimized version of Cas9) used in this study are both available through the non-profit plasmid distribution service Addgene (addgene.org/crispr-cas).

### EGFP Activity Assays

U2OS.EGFP cells harboring a single integrated copy of an EGFP-PEST fusion gene were cultured as previously described (Reyon et al., Nat Biotech 30, 460-465 (2012)). For transfections, 200,000 cells were Nucleofected with the indicated amounts of gRNA expression plasmid and pJDS246 together with 30 ng of a Td-tomato-encoding plasmid using the SE Cell Line 4D-Nucleofector™ X Kit (Lonza) according to the manufacturer's protocol. Cells were analyzed 2 days post-transfection using a BD LSRII flow cytometer. Transfections for optimizing gRNA/Cas9 plasmid concentration were performed in triplicate and all other transfections were performed in duplicate.

### PCR Amplification and Sequence Verification of Endogenous Human Genomic Sites

PCR reactions were performed using Phusion Hot Start II high-fidelity DNA polymerase (NEB) with PCR primers and conditions listed in Table B. Most loci amplified successfully using touchdown PCR (98° C., 10 s: 72-62° C., −1° C./cycle, 15 s: 72° C., 30 s]10 cycles, [98° C., 10 s: 62° ° C., 15 s: 72° C., 30 s]25 cycles). PCR for the remaining targets were performed with 35 cycles at a constant annealing temperature of 68° C. or 72° ° C. and 3% DMSO or IM betaine, if necessary. PCR products were analyzed on a QIAXCEL capillary electrophoresis system to verify both size and purity. Validated products were treated with ExoSap-IT (Affymetrix) and sequenced by the Sanger method (MGH DNA Sequencing Core) to verify each target site.

### Determination of RGN-Induced On- and Off-Target Mutation Frequencies in Human Cells

For U20S.EGFP and K562 cells, 2×10⁵ cells were transfected with 250 ng of gRNA expression plasmid or an empty U6 promoter plasmid (for negative controls), 750 ng of Cas9 expression plasmid, and 30 ng of td-Tomato expression plasmid using the 4D Nucleofector System according to the manufacturer's instructions (Lonza). For HEK293 cells, 1.65×10⁵ cells were transfected with 125 ng of gRNA expression plasmid or an empty U6 promoter plasmid (for the negative control), 375 ng of Cas9 expression plasmid, and 30 ng of a td-Tomato expression plasmid using Lipofectamine LTX reagent according to the manufacturer's instructions (Life Technologies). Genomic DNA was harvested from transfected U2OS.EGFP, HEK293, or K562 cells using the QIAamp DNA Blood Mini Kit (QIAGEN), according to the manufacturer's instructions. To generate enough genomic DNA to amplify the off-target candidate sites, DNA from three Nucleofections (for U2OS.EGFP cells), two Nucleofections (for K562 cells), or two Lipofectamine LTX transfections was pooled together before performing T7EI. This was done twice for each condition tested, thereby generating duplicate pools of genomic DNA representing a total of four or six individual transfections. PCR was then performed using these genomic DNAs as templates as described above and purified using Ampure XP beads (Agencourt) according to the manufacturer's instructions. T7EI assays were performed as previously described (Reyon et al., 2012, supra).

### DNA Sequencing of NHEJ-Mediated Indel Mutations

Purified PCR products used for the T7EI assay were cloned into Zero Blunt TOPO vector (Life Technologies) and plasmid DNAs were isolated using an alkaline lysis miniprep method by the MGH DNA Automation Core. Plasmids were sequenced using an M13 forward primer (5′-GTAAAACGACGGCCAG-3′ (SEQ ID NO:35)) by the Sanger method (MGH DNA Sequencing Core).

### Example 1a. Single Nucleotide Mismatches

To begin to define the specificity determinants of RGNs in human cells, a large-scale test was performed to assess the effects of systematically mismatching various positions within multiple gRNA/target DNA interfaces. To do this, a quantitative human cell-based enhanced green fluorescent protein (EGFP) disruption assay previously described (see Methods above and Reyon et al., 2012, supra) that enables rapid quantitation of targeted nuclease activities (FIG. 2B) was used. In this assay, the activities of nucleases targeted to a single integrated EGFP reporter gene can be quantified by assessing loss of fluorescence signal in human U2OS.EGFP cells caused by inactivating frameshift insertion/deletion (indel) mutations introduced by error prone non-homologous end-joining (NHEJ) repair of nuclease-induced double-stranded breaks (DSBs) (FIG. 2B). For the studies described here, three ˜100 nt single gRNAs (gRNAs) targeted to different sequences within EGFP were used, as follows:

EGFP Site 1
SEQ ID NO: 1
GGGCACGGGCAGCTTGCCGGTGG
EGFP Site 2
SEQ ID NO: 2
GATGCCGTTCTTCTGCTTGTCGG
EGFP Site 3
SEQ ID NO: 3
GGTGGTGCAGATGAACTTCAGGG

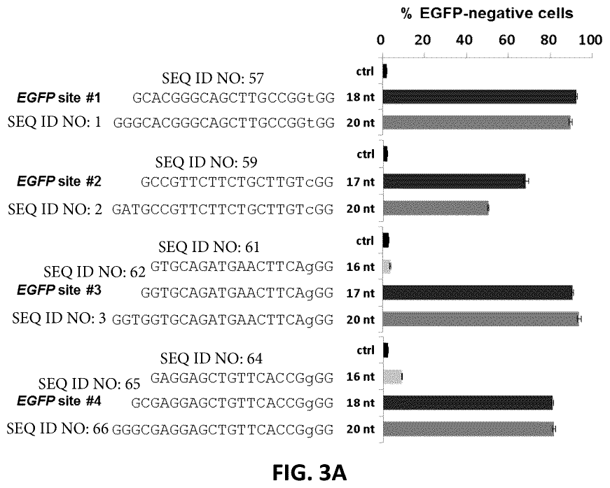

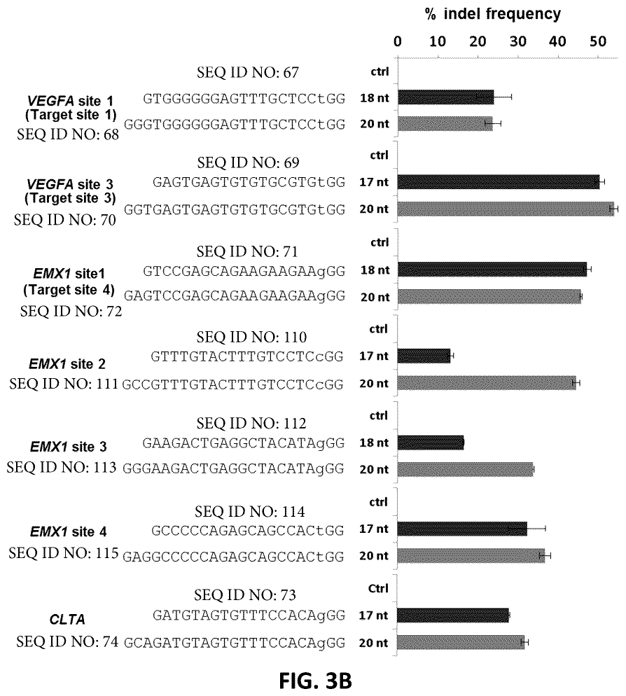

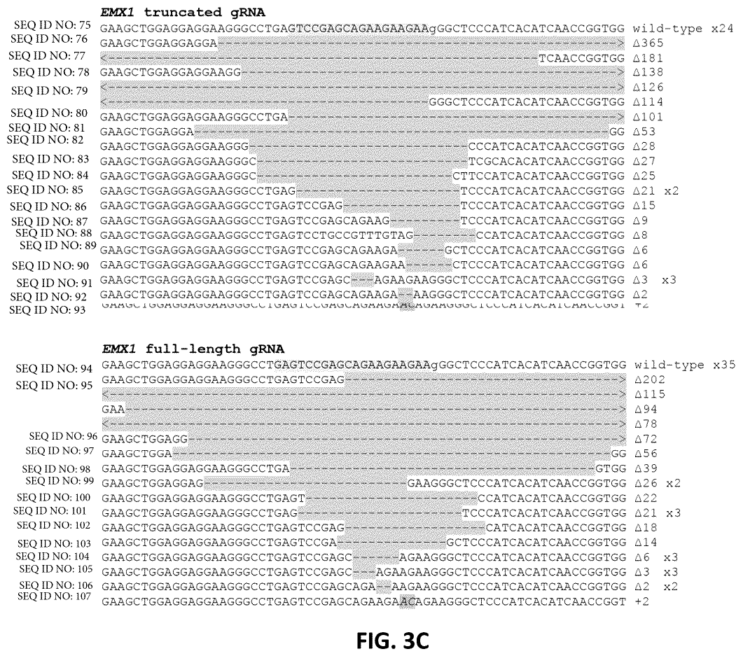

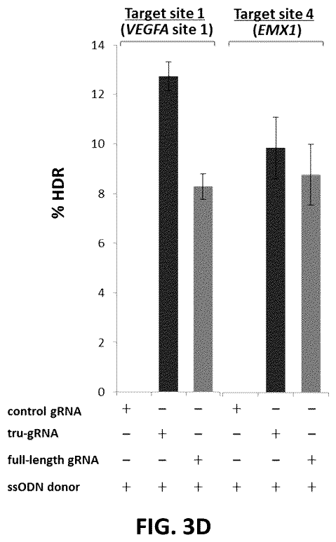

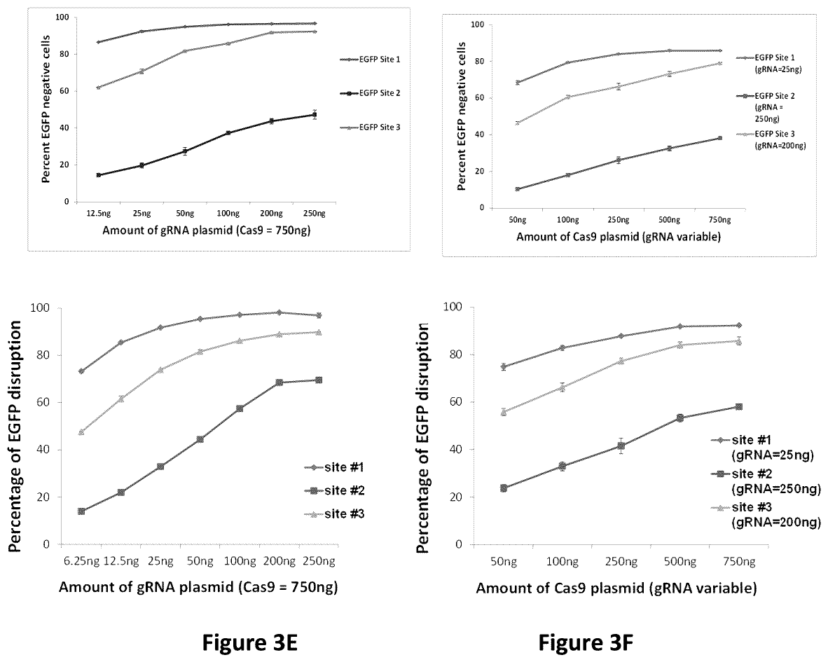

Each of these gRNAs can efficiently direct Cas9-mediated disruption of EGFP expression (see Example 1e and 2a, and FIGS. 3E (top) and 3F (top)).

In initial experiments, the effects of single nucleotide mismatches at 19 of 20 nucleotides in the complementary targeting region of three EGFP-targeted gRNAs were tested. To do this, variant gRNAs were generated for each of the three target sites harboring Watson-Crick transversion mismatches at positions 1 through 19 (numbered 1 to 20 in the 3′ to 5′ direction: see FIG. 1) and the abilities of these various gRNAs to direct Cas9-mediated EGFP disruption in human cells tested (variant gRNAs bearing a substitution at position 20 were not generated because this nucleotide is part of the U6 promoter sequence and therefore must remain a guanine to avoid affecting expression.)

For EGFP target site #2, single mismatches in positions 1-10 of the gRNA have dramatic effects on associated Cas9 activity (FIG. 2C, middle panel), consistent with previous studies that suggest mismatches at the 5′ end of gRNAs are better tolerated than those at the 3′ end (Jiang et al., Nat Biotechnol 31, 233-239 (2013); Cong et al., Science 339, 819-823 (2013); Jinek et al., Science 337, 816-821 (2012)). However, with EGFP target sites #1 and #3, single mismatches at all but a few positions in the gRNA appear to be well tolerated, even within the 3′ end of the sequence. Furthermore, the specific positions that were sensitive to mismatch differed for these two targets (FIG. 2C, compare top and bottom panels)—for example, target site #1 was particularly sensitive to a mismatch at position 2 whereas target site #3 was most sensitive to mismatches at positions 1 and 8.

### Example 1b. Multiple Mismatches

To test the effects of more than one mismatch at the gRNA/DNA interface, a series of variant gRNAs bearing double Watson-Crick transversion mismatches in adjacent and separated positions were created and the abilities of these gRNAs to direct Cas9 nuclease activity were tested in human cells using the EGFP disruption assay. All three target sites generally showed greater sensitivity to double alterations in which one or both mismatches occur within the 3′ half of the gRNA targeting region. However, the magnitude of these effects exhibited site-specific variation, with target site #2 showing the greatest sensitivity to these double mismatches and target site #1 generally showing the least. To test the number of adjacent mismatches that can be tolerated, variant gRNAs were constructed bearing increasing numbers of mismatched positions ranging from positions 19 to 15 in the 5′ end of the gRNA targeting region (where single and double mismatches appeared to be better tolerated).

Testing of these increasingly mismatched gRNAs revealed that for all three target sites, the introduction of three or more adjacent mismatches results in significant loss of RGN activity. A sudden drop off in activity occurred for three different EGFP-targeted gRNAs as one makes progressive mismatches starting from position 19 in the 5′ end and adding more mismatches moving toward the 3′ end. Specifically, gRNAs containing mismatches at positions 19 and 19+18 show essentially full activity whereas those with mismatches at positions 19+18+17, 19+18+17+16, and 19+18+17+16+15 show essentially no difference relative to a negative control (FIG. 2F). (Note that we did not mismatch position 20 in these variant gRNAs because this position needs to remain as a G because it is part of the U6 promoter that drives expression of the gRNA.)

Additional proof of that shortening gRNA complementarity might lead to RGNs with greater specificities was obtained in the following experiment: for four different EGFP-targeted gRNAs (FIG. 2H), introduction of a double mismatch at positions 18 and 19 did not significantly impact activity. However, introduction of another double mismatch at positions 10 and 11 then into these gRNAs results in near complete loss of activity. Interestingly introduction of only the 10/11 double mismatches does not generally have as great an impact on activity.

Taken together, these results in human cells confirm that the activities of RGNs can be more sensitive to mismatches in the 3′ half of the gRNA targeting sequence. However, the data also clearly reveal that the specificity of RGNs is complex and target site-dependent, with single and double mismatches often well tolerated even when one or more mismatches occur in the 3′ half of the gRNA targeting region. Furthermore, these data also suggest that not all mismatches in the 5′ half of the gRNA/DNA interface are necessarily well tolerated.

In addition, these results strongly suggest that gRNAs bearing shorter regions of complementarity (specifically ˜17 nts) will be more specific in their activities. We note that 17 nts of specificity combined with the 2 nts of specificity conferred by the PAM sequence results in specification of a 19 bp sequence, one of sufficient length to be unique in large complex genomes such as those found in human cells.

### Example 1c. Off-Target Mutations

To determine whether off-target mutations for RGNs targeted to endogenous human genes could be identified, six gRNAs that target three different sites in the VEGFA gene, one in the EMX1 gene, one in the RNF2 gene, and one in the FANCF gene were used. These six gRNAs efficiently directed Cas9-mediated indels at their respective endogenous loci in human U2OS.EGFP cells as detected by T7 Endonuclease I (T7EI) assay. For each of these six RGNs, we then examined dozens of potential off-target sites (ranging in number from 46 to as many as 64) for evidence of nuclease-induced NHEJ-mediated indel mutations in U2OS.EGFP cells. The loci assessed included all genomic sites that differ by one or two nucleotides as well as subsets of genomic sites that differ by three to six nucleotides and with a bias toward those that had one or more of these mismatches in the 5′ half of the gRNA targeting sequence. Using the T7EI assay, four off-target sites (out of 53 candidate sites examined) for VEGFA site 1, twelve (out of 46 examined) for VEGFA site 2, seven (out of 64 examined) for VEGFA site 3 and one (out of 46 examined) for the EMX1 site were readily identified. No off-target mutations were detected among the 43 and 50 potential sites examined for the RNF2 or FANCF genes, respectively. The rates of mutation at verified off-target sites were very high, ranging from 5.6% to 125% (mean of 40%) of the rate observed at the intended target site. These bona fide off-targets included sequences with mismatches in the 3′ end of the target site and with as many as a total of five mismatches, with most off-target sites occurring within protein coding genes. DNA sequencing of a subset of off-target sites provided additional molecular confirmation that indel mutations occur at the expected RGN cleavage site.

### Example 1d. Off-Target Mutations in Other Cell Types

Having established that RGNs can induce off-target mutations with high frequencies in U2OS.EGFP cells, we next sought to determine whether these nucleases would also have these effects in other types of human cells. We had chosen U2OS.EGFP cells for our initial experiments because we previously used these cells to evaluate the activities of TALENs15 but human HEK293 and K562 cells have been more widely used to test the activities of targeted nucleases. Therefore, we also assessed the activities of the four RGNs targeted to VEGFA sites 1, 2, and 3 and the EMX1 site in HEK293 and K562 cells. We found that each of these four RGNs efficiently induced NHEJ-mediated indel mutations at their intended on-target site in these two additional human cell lines (as assessed by T7EI assay), albeit with somewhat lower mutation frequencies than those observed in U2OS.EGFP cells. Assessment of the 24 off-target sites for these four RGNs originally identified in U2OS.EGFP cells revealed that many were again mutated in HEK293 and K562 cells with frequencies similar to those at their corresponding on-target site. DNA sequencing of a subset of these off-target sites from HEK293 cells provided additional molecular evidence that alterations are occurring at the expected genomic loci. We do not know for certain why in HEK293 cells four and in K562 cells eleven of the off-target sites identified in U2OS.EGFP cells did not show detectable mutations. However, we note that many of these off-target sites also showed relatively lower mutation frequencies in U2OS.EGFP cells. Therefore, we speculate that mutation rates of these sites in HEK293 and K562 cells may be falling below the reliable detection limit of our T7EI assay (˜2-5%) because RGNs generally appear to have lower activities in HEK293 and K562 cells compared with U2OS.EGFP cells in our experiments. Taken together, our results in HEK293 and K562 cells provide evidence that the high-frequency off-target mutations we observe with RGNs will be a general phenomenon seen in multiple human cell types.

### Example 1e. Titration of gRNA- and Cas9-Expressing Plasmid Amounts Used for the EGFP Disruption Assay

Single guide RNAs (gRNAs) were generated for three different sequences (EGFP SITES 1-3, shown above) located upstream of EGFP nucleotide 502, a position at which the introduction of frameshift mutations via non-homologous end-joining can robustly disrupt expression of EGFP (Maeder, M. L. et al., Mol Cell 31, 294-301 (2008); Reyon, D. et al., Nat Biotech 30, 460-465 (2012)).

For each of the three target sites, a range of gRNA-expressing plasmid amounts (12.5 to 250 ng) was initially transfected together with 750 ng of a plasmid expressing a codon-optimized version of the Cas9 nuclease into our U20S.EGFP reporter cells bearing a single copy, constitutively expressed EGFP-PEST reporter gene. All three RGNs efficiently disrupted EGFP expression at the highest concentration of gRNA plasmid (250 ng) (FIG. 3E (top)). However, RGNs for target sites #1 and #3 exhibited equivalent levels of disruption when lower amounts of gRNA-expressing plasmid were transfected whereas RGN activity at target site #2 dropped immediately when the amount of gRNA-expressing plasmid transfected was decreased (FIG. 3E (top)).

The amount of Cas9-encoding plasmid (range from 50 ng to 750 ng) transfected into our U2OS.EGFP reporter cells was titrated EGFP disruption assayed. As shown in FIG. 3F (top), target site #1 tolerated a three-fold decrease in the amount of Cas9-encoding plasmid transfected without substantial loss of EGFP disruption activity. However, the activities of RGNs targeting target sites #2 and #3 decreased immediately with a three-fold reduction in the amount of Cas9 plasmid transfected (FIG. 3F (top)). Based on these results, 25 ng/250 ng, 250 ng/750 ng, and 200 ng/750 ng of gRNA-/Cas9-expressing plasmids were used for EGFP target sites #1, #2, and #3, respectively, for the experiments described in Examples 1a-1d.

The reasons why some gRNA/Cas9 combinations work better than others in disrupting EGFP expression is not understood, nor is why some of these combinations are more or less sensitive to the amount of plasmids used for transfection. Although it is possible that the range of off-target sites present in the genome for these three gRNAs might influence each of their activities, no differences were seen in the numbers of genomic sites that differ by one to six bps for each of these particular target sites that would account for the differential behavior of the three gRNAs.

### Example 2: Using guideRNAs Containing Csy4 Binding Sites with Cas9

In this example, dCas9 is expressed together with a modified gRNA bearing extra RNA sequence on either or both of the 5′ and/or 3′ end of the gRNA that is bound by Csy4, an RNA-binding protein, as well as a fusion protein with Csy4 fused to the FokI nuclease domain. As shown in FIG. 6, two dCas9 molecules would be targeted to adjacent DNA sequences by appropriate gRNAs and the Csy4-binding sequence on the two gRNA would interact with the Csy4-FokI nuclease domain fusion proteins. The Fok nuclease domains would dimerize, resulting in introduction of a targeted double-stranded break in the DNA sequence between the two dCas9 binding sites.

Thus, Csy4 RNA binding sites were attached to the 3′ and 5′ ends of a gRNA sequence and expressed with Cas9 in cells. The Csy4 RNA binding site sequence ‘GUUCACUGCCGUAUAGGCAGCUAAGAAA (SEQ ID NO:36)’ was fused to the 5′ and 3′ end of the standard gRNA sequence.

Multiplex gRNA encoding plasmids were constructed by ligating: 1) annealed oligos encoding the first target site, 2) phosphorylated annealed oligos encoding crRNA, tracrRNA, and Csy4-binding site, and 3) annealed oligos encoding the second targetsite, into a U6-Csy4site-gRNA plasmid backbone digested with BsmBI Type IIs restriction enzyme.

(SEQ ID NO: 37)
GUUCACUGCCGUAUAGGCAGNNNNNNNNNNNNNNNNNNNNGUUUUAGAG
CUAGAAAUAGCAAGUUAAAAUAAGGCUAGUCCGUUAUCAACUUGAAAAA
GUGGCACCGAGUCGGUGCGUUCACUGCCGUAUAGGCAGNNNNNNNNNNN
NNNNNNNNNGUUUUAGAGCUAGAAAUAGCAAGUUAAAAUAAGGCUAGUC
CGUUAUCAACUUGAAAAAGUGGCACCGAGUCGGUGCGUUCACUGCCGUA
UAGGCAG

This sequence is a multiplex gRNA sequence flanked by Csy4 sites (underlined). When processed by Csy4FokI, Csy4FokI remains bound. Functionally, encoding these in multiplex on one transcript should have the same result as encoding them separately. Although all pairs of Csy4-flanked gRNAs were expressed in a multiplex context in the experiments described herein, the gRNAs can be encoded in multiplex gRNAs separated by Csy4 sites encoded on one transcript as well as individual gRNAs that have an additional Csy4 sequence. In this sequence, the first N20 sequence represents the sequence complementary to one strand of the target genomic sequence, and the second N20 sequence represents the sequence complementary to the other strand of the target genomic sequence.

A plasmid encoding the Csy4 recognition site containing gRNA was co-transfected with plasmid encoding Cas9 and Csy4 proteins separated by a ‘2A’ peptide linkage. The results showed that gRNAs with Csy4 sites fused to the 5′ and 3′ ends remained capable of directing Cas9-mediated cleavage in human cells using the U2OS-EGFP disruption assay previously described. Thus, Csy4 RNA binding sites can be attached to 3′ end of a gRNA sequence and complexes of these Csy4 site-containing gRNAs with Cas9 remain functional in the cell (FIG. 7A).

Additional experiments were performed to demonstrate that co-expression of two gRNAs targeted to adjacent sites on a DNA sequence and harboring a Csy4 binding site on their 3′ ends, dCas9 protein, and a Csy4-FokI fusion in human cells can lead to cleavage and subsequent mutagenesis of the DNA between the two gRNA binding sites.

The sequences of the Csy4-FokI fusion proteins were as follows:

Csy4-FokI N-terminal fusion (nucleotide sequence)
(SEQ ID NO: 38)
ATGGACCACTACCTCGACATTCGCTTGCGACCGGACCCGGAATTTCCCC
CGGCGCAACTCATGAGCGTGCTCTTCGGCAAGCTCCACCAGGCCCTGGT
GGCACAGGGCGGGGACAGGATCGGCGTGAGCTTCCCCGACCTCGACGAA
AGCCGCTCCCGGCTGGGCGAGCGCCTGCGCATTCATGCCTCGGCGGACG
ACCTTCGTGCCCTGCTCGCCCGGCCCTGGCTGGAAGGGTTGCGGGACCA
TCTGCAATTCGGAGAACCGGCAGTCGTGCCTCACCCCACACCGTACCGT
CAGGTCAGTCGGGTTCAGGCGAAAAGCAATCCGGAACGCCTGCGGCGGC
GGCTCATGCGCCGGCACGATCTGAGTGAGGAGGAGGCTCGGAAACGCAT
TCCCGATACGGTCGCGAGAGCCTTGGACCTGCCCTTCGTCACGCTACGC
AGCCAGAGCACCGGACAGCACTTCCGTCTCTTCATCCGCCACGGGCCGT
TGCAGGTGACGGCAGAGGAAGGAGGATTCACCTGTTACGGGTTGAGCAA
AGGAGGTTTCGTTCCCTGGTTCGGTGGCGGTGGATCCCAACTAGTCAAA
AGTGAACTGGAGGAGAAGAAATCTGAACTTCGTCATAAATTGAAATATG
TGCCTCATGAATATATTGAATTAATTGAAATTGCCAGAAATTCCACTCA
GGATAGAATTCTTGAAATGAAGGTAATGGAATTTTTTATGAAAGTTTAT
GGATATAGAGGTAAACATTTGGGTGGATCAAGGAAACCGGACGGAGCAA
TTTATACTGTCGGATCTCCTATTGATTACGGTGTGATCGTGGATACTAA
AGCTTATAGCGGAGGTTATAATCTGCCAATTGGCCAAGCAGATGAAATG
CAACGATATGTCGAAGAAAATCAAACACGAAACAAACATATCAACCCTA
ATGAATGGTGGAAAGTCTATCCATCTTCTGTAACGGAATTTAAGTTTTT
ATTTGTGAGTGGTCACTTTAAAGGAAACTACAAAGCTCAGCTTACACGA
TTAAATCATATCACTAATTGTAATGGAGCTGTTCTTAGTGTAGAAGAGC
TTTTAATTGGTGGAGAAATGATTAAAGCCGGCACATTAACCTTAGAGGA
AGTCAGACGGAAATTTAATAACGGCGAGATAAACTTTTGA
Csy4-FokI N-terminal fusion (amino acid sequence,
GGGGS linker underlined)
(SEQ ID NO: 39)
MDHYLDIRLRPDPEFPPAQLMSVLFGKLHQALVAQGGDRIGVSFPDLDE
SRSRLGERLRIHASADDLRALLARPWLEGLRDHLQFGEPAVVPHPTPYR
QVSRVQAKSNPERLRRRLMRRHDLSEEEARKRIPDTVARALDLPFVTLR
SQSTGQHFRLFIRHGPLQVTAEEGGFTCYGLSKGGFVPWFGGGGSQLVK
SELEEKKSELRHKLKYVPHEYIELIEIARNSTQDRILEMKVMEFFMKVY
GYRGKHLGGSRKPDGAIYTVGSPIDYGVIVDTKAYSGGYNLPIGQADEM
QRYVEENQTRNKHINPNEWWKVYPSSVTEFKFLFVSGHFKGNYKAQLTR
LNHITNCNGAVLSVEELLIGGEMIKAGTLTLEEVRRKFNNGEINF*
Csy4-FokI C-terminal fusion (nucleotide sequence)
(SEQ ID NO: 40)
ATGCAACTAGTCAAAAGTGAACTGGAGGAGAAGAAATCTGAACTTCGTC
ATAAATTGAAATATGTGCCTCATGAATATATTGAATTAATTGAAATTGC
CAGAAATTCCACTCAGGATAGAATTCTTGAAATGAAGGTAATGGAATTT
TTTATGAAAGTTTATGGATATAGAGGTAAACATTTGGGTGGATCAAGGA
AACCGGACGGAGCAATTTATACTGTCGGATCTCCTATTGATTACGGTGT
GATCGTGGATACTAAAGCTTATAGCGGAGGTTATAATCTGCCAATTGGC
CAAGCAGATGAAATGCAACGATATGTCGAAGAAAATCAAACACGAAACA
AACATATCAACCCTAATGAATGGTGGAAAGTCTATCCATCTTCTGTAAC
GGAATTTAAGTTTTTATTTGTGAGTGGTCACTTTAAAGGAAACTACAAA
GCTCAGCTTACACGATTAAATCATATCACTAATTGTAATGGAGCTGTTC
TTAGTGTAGAAGAGCTTTTAATTGGTGGAGAAATGATTAAAGCCGGCAC
ATTAACCTTAGAGGAAGTCAGACGGAAATTTAATAACGGCGAGATAAAC
TTTGGTGGCGGTGGATCCGACCACTACCTCGACATTCGCTTGCGACCGG
ACCCGGAATTTCCCCCGGCGCAACTCATGAGCGTGCTCTTCGGCAAGCT
CCACCAGGCCCTGGTGGCACAGGGCGGGGACAGGATCGGCGTGAGCTTC
CCCGACCTCGACGAAAGCCGCTCCCGGCTGGGCGAGCGCCTGCGCATTC
ATGCCTCGGCGGACGACCTTCGTGCCCTGCTCGCCCGGCCCTGGCTGGA
AGGGTTGCGGGACCATCTGCAATTCGGAGAACCGGCAGTCGTGCCTCAC
CCCACACCGTACCGTCAGGTCAGTCGGGTTCAGGCGAAAAGCAATCCGG
AACGCCTGCGGCGGCGGCTCATGCGCCGGCACGATCTGAGTGAGGAGGA
GGCTCGGAAACGCATTCCCGATACGGTCGCGAGAGCCTTGGACCTGCCC
TTCGTCACGCTACGCAGCCAGAGCACCGGACAGCACTTCCGTCTCTTCA
TCCGCCACGGGCCGTTGCAGGTGACGGCAGAGGAAGGAGGATTCACCTG
TTACGGGTTGAGCAAAGGAGGTTTCGTTCCCTGGTTCTGA
Csy4-FokI C-terminal fusion (amino acid sequence,
GGGGS linker underlined)
(SEQ ID NO: 41)
MQLVKSELEEKKSELRHKLKYVPHEYIELIEIARNSTQDRILEMKVMEF
FMKVYGYRGKHLGGSRKPDGAIYTVGSPIDYGVIVDTKAYSGGYNLPIG
QADEMQRYVEENQTRNKHINPNEWWKVYPSSVTEFKFLFVSGHFKGNYK
AQLTRLNHITNCNGAVLSVEELLIGGEMIKAGTLTLEEVRRKFNNGEIN
FGGGGSDHYLDIRLRPDPEFPPAQLMSVLFGKLHQALVAQGGDRIGVSF
PDLDESRSRLGERLRIHASADDLRALLARPWLEGLRDHLQFGEPAVVPH
PTPYRQVSRVQAKSNPERLRRRLMRRHDLSEEEARKRIPDTVARALDLP
FVTLRSQSTGQHFRLFIRHGPLQVTAEEGGFTCYGLSKGGFVPWF*

Because the orientation and geometry of the gRNA/dCas9/Csy4-FokI complexes required to induce a targeted DSB is not known, we performed a series of experiments designed to ascertain these parameters. For these experiments, we utilized a human cell-based EGFP disruption assay in which introduction of a targeted DSB into the coding sequence of a single integrated EGFP gene leads to the introduction of indel mutations and disruption of functional EGFP expression. Thus, the percentage of EGFP-negative cells, which can be quantified by flow cytometry, serves as a surrogate measure of targeted nuclease activity. To optimize parameters, we identified a large series of paired gRNA target sites that varied in the spacer length between the two sites (edge-to-edge distance between the N20NGG target sites). In addition, the orientation of the gRNA target sites were such that they either had their PAM sequences oriented “outward” from the spacer sequence in between or “inward” towards the spacer sequence in between. We expressed pairs of gRNAs targeted to these sites in our human EGFP reporter cell line together with dCas9 protein and either (a) a fusion of FokI nuclease domain fused to the amino-terminal end of Csy4 (FokI-Csy4 fusion protein) or (b) a fusion of FokI nuclease domain fused to the carboxy-terminal end of Csy4 (Csy4-FokI fusion protein) and then assessed by flow cytometry the efficiencies with which these combinations could induce EGFP-negative cells.

These experiments demonstrate that the FokI-Csy4 fusion proteins were most robustly active in concert with dCas9 and pairs of gRNA for sites in which the PAM sequences were oriented “outward” with spacer distances of 15-16 bp (FIG. 7C and data not shown).

Interestingly, there are also more moderate potential peaks of activity at spacer distances of 22 and 25 bps on the “outward” oriented sites. No activity was observed for the Csy4-FokI fusions on any of the “outward” oriented sites nor was any activity observed with either FokI-Csy4 or Csy4-FokI proteins for any pairs of sites in which the PAM sequences were oriented “inward” (data not shown). T7 endonuclease I assays demonstrated that the indel mutations induced by the gRNA/dCas9/FokI-Csy4 complexes were targeted to the expected location within the EGFP coding sequence (FIG. 7D). Thus, this configuration (depicted in FIG. 7B) enables gRNA/dCas9/FokI-Csy4 complexes to induce specific cleavage of DNA sequences that requires two gRNA binding sites, thereby increasing the specificity of the cleavage event.

### REFERENCES

Cheng, A. W., Wang, H., Yang, H., Shi, L., Katz, Y., Theunissen, T. W., Rangarajan, S., Shivalila, C. S., Dadon, D. B., and Jaenisch, R. Multiplexed activation of endogenous genes by CRISPR-on, an RNA-guided transcriptional activator system. Cell Res 23, 1163-1171. (2013).
Cho, S. W., Kim, S., Kim, J. M. & Kim, J. S. Targeted genome engineering in human cells with the Cas9 RNA-guided endonuclease. Nat Biotechnol 31, 230-232 (2013).
Cong, L. et al. Multiplex genome engineering using CRISPR/Cas systems. Science 339, 819-823 (2013).
Cradick, T. J., Fine, E. J., Antico, C. J., and Bao, G. CRISPR/Cas9 systems targeting beta-globin and CCR5 genes have substantial off-target activity. Nucleic Acids Res. (2013).
Dicarlo, J. E. et al. Genome engineering in Saccharomyces cerevisiae using CRISPR-Cas systems. Nucleic Acids Res (2013).
Ding, Q., Regan, S. N., Xia, Y., Oostrom, L. A., Cowan, C. A., and Musunuru, K. Enhanced efficiency of human pluripotent stem cell genome editing through replacing TALENs with CRISPRs. Cell Stem Cell 12, 393-394. (2013).
Fisher, S., Barry, A., Abreu, J., Minie, B., Nolan, J., Delorey, T. M., Young, G., Fennell, T. J., Allen, A., Ambrogio, L., et al. A scalable, fully automated process for construction of sequence-ready human exome targeted capture libraries. Genome Biol 12, R1. (2011).
Friedland, A. E., Tzur, Y. B., Esvelt, K. M., Colaiacovo, M. P., Church, G. M., and
Calarco, J. A. Heritable genome editing in C. elegans via a CRISPR-Cas9 system. Nat Methods 10, 741-743. (2013).
Fu, Y., Foden, J. A., Khayter, C., Maeder, M. L., Reyon, D., Joung, J. K., and Sander, J. D. High-frequency off-target mutagenesis induced by CRISPR-Cas nucleases in human cells. Nat Biotechnol 31, 822-826. (2013).
Gabriel, R. et al. An unbiased genome-wide analysis of zinc-finger nuclease specificity. Nat Biotechnol 29, 816-823 (2011).
Gilbert, L. A., Larson, M. H., Morsut, L., Liu, Z., Brar, G. A., Torres, S. E., Stern-Ginossar, N., Brandman, O., Whitehead, E. H., Doudna, J. A., et al. (2013). CRISPR-Mediated Modular RNA-Guided Regulation of Transcription in Eukaryotes. Cell 154, 442-451.
Gratz, S. J. et al. Genome engineering of Drosophila with the CRISPR RNA-guided Cas9 nuclease. Genetics (2013).
Hockemeyer, D. et al. Genetic engineering of human pluripotent cells using TALE nucleases. Nat Biotechnol 29, 731-734 (2011).
Horvath, P. & Barrangou, R. CRISPR/Cas, the immune system of bacteria and archaea. Science 327, 167-170 (2010).
Hsu, P. D., Scott, D. A., Weinstein, J. A., Ran, F. A., Konermann, S., Agarwala, V., Li, Y., Fine, E. J., Wu, X., Shalem, O., et al. DNA targeting specificity of RNA-guided Cas9 nucleases. Nat Biotechnol 31, 827-832. (2013).
Hwang, W. Y. et al. Efficient genome editing in zebrafish using a CRISPR-Cas system. Nat Biotechnol 31, 227-229 (2013).
Hwang, W. Y., Fu, Y., Reyon, D., Maeder, M. L., Kaini, P., Sander, J. D., Joung, J. K., Peterson, R. T., and Yeh, J. R. Heritable and Precise Zebrafish Genome Editing Using a CRISPR-Cas System. PLOS One 8, e68708. (2013a).
Jiang, W., Bikard, D., Cox, D., Zhang, F. & Marraffini, L. A. RNA-guided editing of bacterial genomes using CRISPR-Cas systems. Nat Biotechnol 31, 233-239 (2013).
Jinek, M. et al. A programmable dual-RNA-guided DNA endonuclease in adaptive bacterial immunity. Science 337, 816-821 (2012).
Jinek, M. et al. RNA-programmed genome editing in human cells. Elife 2, e00471 (2013).
Li, D., Qiu, Z., Shao, Y., Chen, Y., Guan, Y., Liu, M., Li, Y., Gao, N., Wang, L., Lu,
X., et al. Heritable gene targeting in the mouse and rat using a CRISPR-Cas system. Nat Biotechnol 31, 681-683. (2013a).
Li, W., Teng, F., Li, T., and Zhou, Q. Simultaneous generation and germline transmission of multiple gene mutations in rat using CRISPR-Cas systems. Nat Biotechnol 31, 684-686. (2013b).
Maeder, M. L., Linder, S. J., Cascio, V. M., Fu, Y., Ho, Q. H., and Joung, J. K. CRISPR RNA-guided activation of endogenous human genes. Nat Methods 10, 977-979. (2013).
Mali, P., Aach, J., Stranges, P. B., Esvelt, K. M., Moosburner, M., Kosuri, S., Yang, L., and Church, G. M. CAS9 transcriptional activators for target specificity screening and paired nickases for cooperative genome engineering. Nat Biotechnol 31, 833-838. (2013a).
Mali, P., Esvelt, K. M., and Church, G. M. Cas9 as a versatile tool for engineering biology. Nat Methods 10, 957-963. (2013b).
Mali, P. et al. RNA-guided human genome engineering via Cas9. Science 339, 823-826 (2013c).
Pattanayak, V., Lin, S., Guilinger, J. P., Ma, E., Doudna, J. A., and Liu, D. R. High-throughput profiling of off-target DNA cleavage reveals RNA-programmed Cas9 nuclease specificity. Nat Biotechnol 31, 839-843. (2013).
Pattanayak, V., Ramirez, C. L., Joung, J. K. & Liu, D. R. Revealing off-target cleavage specificities of zinc-finger nucleases by in vitro selection. Nat Methods 8, 765-770 (2011).
Perez, E. E. et al. Establishment of HIV-1 resistance in CD4+ T cells by genome editing using zinc-finger nucleases. Nat Biotechnol 26, 808-816 (2008).
Perez-Pinera, P., Kocak, D. D., Vockley, C. M., Adler, A. F., Kabadi, A. M., Polstein, L. R., Thakore, P. I., Glass, K. A., Ousterout, D. G., Leong, K. W., et al. RNA-guided gene activation by CRISPR-Cas9-based transcription factors. Nat Methods 10, 973-976. (2013).
Qi, L. S., Larson, M. H., Gilbert, L. A., Doudna, J. A., Weissman, J. S., Arkin, A. P., and Lim, W. A. Repurposing CRISPR as an RNA-guided platform for sequence-specific control of gene expression. Cell 152, 1173-1183. (2013).
Ran, F. A., Hsu, P. D., Lin, C. Y., Gootenberg, J. S., Konermann, S., Trevino, A. E., Scott, D. A., Inoue, A., Matoba, S., Zhang, Y., et al. Double nicking by RNA-guided CRISPR Cas9 for enhanced genome editing specificity. Cell 154, 1380-1389. (2013). Reyon, D. et al. FLASH assembly of TALENs for high-throughput genome editing. Nat Biotech 30, 460-465 (2012).
Sander, J. D., Maeder, M. L., Reyon, D., Voytas, D. F., Joung, J. K., and Dobbs, D. ZiFIT (Zinc Finger Targeter); an updated zinc finger engineering tool. Nucleic Acids Res 38, W462-468. (2010).
Sander, J. D., Ramirez, C. L., Linder, S. J., Pattanayak, V., Shoresh, N., Ku, M., Foden, J. A., Reyon, D., Bernstein, B. E., Liu, D. R., et al. In silico abstraction of zinc finger nuclease cleavage profiles reveals an expanded landscape of off-target sites. Nucleic Acids Res. (2013).
Sander, J. D., Zaback, P., Joung, J. K., Voytas, D. F., and Dobbs, D. Zinc Finger Targeter (ZiFiT); an engineered zinc finger/target site design tool. Nucleic Acids Res 35, W599-605. (2007).
Shen, B. et al. Generation of gene-modified mice via Cas9/RNA-mediated gene targeting. Cell Res (2013).
Sugimoto, N. et al. Thermodynamic parameters to predict stability of RNA/DNA hybrid duplexes. Biochemistry 34, 11211-11216 (1995).
Terns, M. P. & Terns, R. M. CRISPR-based adaptive immune systems. Curr Opin Microbiol 14, 321-327 (2011).
Wang, H. et al. One-Step Generation of Mice Carrying Mutations in Multiple Genes by CRISPR/Cas-Mediated Genome Engineering. Cell 153, 910-918 (2013).
Wiedenheft, B., Sternberg, S. H. & Doudna, J. A. RNA-guided genetic silencing systems in bacteria and archaea. Nature 482, 331-338 (2012).
Yang, L., Guell, M., Byrne, S., Yang, J. L., De Los Angeles, A., Mali, P., Aach, J., Kim-Kiselak, C., Briggs, A. W., Rios, X., et al. (2013). Optimization of scarless human stem cell genome editing. Nucleic Acids Res 41, 9049-9061.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-30. (canceled)

31. A tracrRNA comprising one or more deoxyribonucleotides.

32. The tracrRNA of claim 31, wherein the tracrRNA is selected from the group consisting of:
GGAACCAUUCAAAACAGCAUAGCAAGUUAAAAUAAGGCUAGUCCGUUAUC AACUUGAAAAAGUGGCACCGAGUCGGUGC (SEQ ID NO:15) or an active portion thereof;
UAGCAAGUUAAAAUAAGGCUAGUCCGUUAUCAACUUGAAAAAGUGGCACC GAGUCGGUGC (SEQ ID NO:16) or an active portion thereof; or
AGCAUAGCAAGUUAAAAUAAGGCUAGUCCGUUAUCAACUUGAAAAAGUGG CACCGAGUCGGUGC (SEQ ID NO:17) or an active portion thereof, wherein one or more of the nucleotides is a deoxyribonucleic acid.

33. The tracrRNA of claim 31, wherein the tracrRNA molecule is truncated from its 3′ end by at least 10 nt.

34. The tracrRNA of claim 31, wherein the tracrRNA molecule is truncated from its 3′ end by at least 1 nt.

35. The tracr RNA of claim 31, wherein the tracrRNA molecule is truncated from its 5′ end by at least 10 nt.

36. The tracrRNA of claim 31, wherein the tracrRNA molecule is truncated by at least 10 nt on the 5′ end and by at least 1 nt on the 3′ end.

37. A nucleic acid encoding the tracrRNA of claim 31.

38. A vector comprising the nucleic acid of claim 37.

39. A host cell expressing the tracrRNA of claim 31.

40. A composition comprising nucleic acid(s) encoding the tracrRNA of claim 31 and a crRNA.

41. The composition of claim 40 wherein the crRNA comprises a deoxyribonucleic acid.

42. The composition of claim 40, wherein the crRNA comprises: (i) a target complementarity region at the 5′ end; and (ii) a non-complementary region immediately 3′ of the target complementary region.

43. The composition of claim 42, wherein the target complementarity region is from 17 to 25 nucleotides long.

44. The composition of claim 43, wherein the non-complementary region comprises:
GUUUUAGAGCUAUGCUGUUUUG(X_{N});
GUUUUAGAGCUA;
GUUUUAGAGCUAUGCUGUUUUG;
GUUUUAGAGCUAUGCU;
GUUUUAGAGCUAGAAAUAGCAAGUUAAAAUAAGGCUAGUCCG(X_{N});
GUUUUAGAGCUAUGCUGAAAAGCAUAGCAAGUUAAAAUAAGGCUAGUCCG UUAUC(X_{N});
GUUUUAGAGCUAUGCUGUUUUGGAAACAAAACAGCAUAGCAAGUUAAAAU AAGGCUAGUCCGUUAUC(X_{N});
GUUUUAGAGCUAGAAAUAGCAAGUUAAAAUAAGGCUAGUCCGUUAUCAAC UUGAAAAAGUGGCACCGAGUCGGUGC(X_{N}),
GUUUAAGAGCUAGAAAUAGCAAGUUUAAAUAAGGCUAGUCCGUUAUCAAC UUGAAAAAGUGGCACCGAGUCGGUGC;
GUUUUAGAGCUAUGCUGGAAACAGCAUAGCAAGUUUAAAUAAGGCUAGUC CGUUAUCAACUUGAAAAAGUGGCACCGAGUCGGUGC; or
GUUUAAGAGCUAUGCUGGAAACAGCAUAGCAAGUUUAAAUAAGGCUAGUC CGUUAUCAACUUGAAAAAGUGGCACCGAGUCGGUGC, wherein X_{N} is any sequence that does not interfere with the binding of the ribonucleic acid to Cas9.

45. The composition of claim 40, further comprising a nucleic acid encoding a S. pyogenes Cas9 protein or variant thereof.

46. The composition of claim 45, wherein the S. pyogenes Cas9 or variant thereof has at least 90% sequence identity to the amino acid of SEQ ID NO: 18, optionally with mutations at one or more of D10, E762, H983, H840, and N863.

47. The composition of claim 45, wherein the S. pyogenes Cas9 or variant thereof is fused to a heterologous functional domain, with an optional intervening linker.

48. A composition comprising:
a tracrRNA;
a crRNA; and
a S. pyogenes Cas9 protein or variant thereof,
wherein the tracrRNA and/or the crRNA comprise a deoxyribonucleic acid.

49. The composition of claim 48, wherein the tracrRNA comprises a deoxyribonucleic acid.

50. The composition of claim 48, wherein the crRNA comprises a deoxyribonucleic acid.

51. The composition of claim 48, wherein the crRNA and the tracrRNA each comprise a deoxyribonucleic acid.


SEQUENCE LISTING (115 sequences)

SEQ ID NO: 1 - DNA, 23 bp, synthetic construct
  misc_feature 1..23: note = Description of Artificial Sequence: Synthetic binding site oligonucleotide
  source 1..23: mol_type = other DNA
gggcacgggcagcttgccggtgg

SEQ ID NO: 2 - DNA, 23 bp, synthetic construct
  misc_feature 1..23: note = Description of Artificial Sequence: Synthetic binding site oligonucleotide
  source 1..23: mol_type = other DNA
gatgccgttcttctgcttgtcgg

SEQ ID NO: 3 - DNA, 23 bp, synthetic construct
  misc_feature 1..23: note = Description of Artificial Sequence: Synthetic binding site oligonucleotide
  source 1..23: mol_type = other DNA
ggtggtgcagatgaacttcaggg

SEQ ID NO: 4 - RNA, 242 nt, synthetic construct
  misc_feature 1..242: note = Description of Artificial Sequence: Synthetic guide RNA polynucleotide
  source 1..242: mol_type = other RNA
nnnnnnnnnnnnnnnnnnnngttttagagctatgctgttttgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 5 - RNA, 32 nt, synthetic construct
  misc_feature 1..32: note = Description of Artificial Sequence: Synthetic guide RNA oligonucleotide
  source 1..32: mol_type = other RNA
nnnnnnnnnnnnnnnnnnnngttttagagcta

SEQ ID NO: 6 - RNA, 42 nt, synthetic construct
  misc_feature 1..42: note = Description of Artificial Sequence: Synthetic guide RNA oligonucleotide
  source 1..42: mol_type = other RNA
nnnnnnnnnnnnnnnnnnnngttttagagctatgctgttttg

SEQ ID NO: 7 - RNA, 36 nt, synthetic construct
  misc_feature 1..36: note = Description of Artificial Sequence: Synthetic guide RNA oligonucleotide
  source 1..36: mol_type = other RNA
nnnnnnnnnnnnnnnnnnnngttttagagctatgct

SEQ ID NO: 8 - RNA, 262 nt, synthetic construct
  misc_feature 1..262: note = Description of Artificial Sequence: Synthetic guide RNA polynucleotide
  source 1..262: mol_type = other RNA
nnnnnnnnnnnnnnnnnnnngttttagagctagaaatagcaagttaaaataaggctagtccgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 9 - RNA, 275 nt, synthetic construct
  misc_feature 1..275: note = Description of Artificial Sequence: Synthetic guide RNA polynucleotide
  source 1..275: mol_type = other RNA
nnnnnnnnnnnnnnnnnnnngttttagagctatgctgaaaagcatagcaagttaaaataaggctagtccgttatcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 10 - RNA, 287 nt, synthetic construct
  misc_feature 1..287: note = Description of Artificial Sequence: Synthetic guide RNA polynucleotide
  source 1..287: mol_type = other RNA
nnnnnnnnnnnnnnnnnnnngttttagagctatgctgttttggaaacaaaacagcatagcaagttaaaataaggctagtccgttatcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 11 - RNA, 296 nt, synthetic construct
  misc_feature 1..296: note = Description of Artificial Sequence: Synthetic guide RNA polynucleotide
  source 1..296: mol_type = other RNA
nnnnnnnnnnnnnnnnnnnngttttagagctagaaatagcaagttaaaataaggctagtccgttatcaacttgaaaaagtggcaccgagtcggtgcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 12 - RNA, 96 nt, synthetic construct
  misc_feature 1..96: note = Description of Artificial Sequence: Synthetic guide RNA oligonucleotide
  source 1..96: mol_type = other RNA
nnnnnnnnnnnnnnnnnnnngtttaagagctagaaatagcaagtttaaataaggctagtccgttatcaacttgaaaaagtggcaccgagtcggtgc

SEQ ID NO: 13 - RNA, 106 nt, synthetic construct
  misc_feature 1..106: note = Description of Artificial Sequence: Synthetic guide RNA polynucleotide
  source 1..106: mol_type = other RNA
nnnnnnnnnnnnnnnnnnnngttttagagctatgctggaaacagcatagcaagtttaaataaggctagtccgttatcaacttgaaaaagtggcaccgagtcggtgc

SEQ ID NO: 14 - RNA, 106 nt, synthetic construct
  misc_feature 1..106: note = Description of Artificial Sequence: Synthetic guide RNA polynucleotide
  source 1..106: mol_type = other RNA
nnnnnnnnnnnnnnnnnnnngtttaagagctatgctggaaacagcatagcaagtttaaataaggctagtccgttatcaacttgaaaaagtggcaccgagtcggtgc

SEQ ID NO: 15 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

SEQ ID NO: 16 - RNA, 60 nt, synthetic construct
  misc_feature 1..60: note = Description of Artificial Sequence: Synthetic guide RNA oligonucleotide
  source 1..60: mol_type = other RNA
tagcaagttaaaataaggctagtccgttatcaacttgaaaaagtggcaccgagtcggtgc

SEQ ID NO: 17 - RNA, 64 nt, synthetic construct
  misc_feature 1..64: note = Description of Artificial Sequence: Synthetic guide RNA oligonucleotide
  source 1..64: mol_type = other RNA
agcatagcaagttaaaataaggctagtccgttatcaacttgaaaaagtggcaccgagtcggtgc

SEQ ID NO: 18 - protein, 1368 aa, Streptococcus pyogenes [sequence omitted]

SEQ ID NO: 19 - RNA, 23 nt, synthetic construct
  misc_feature 1..23: note = Description of Artificial Sequence: Synthetic binding oligonucleotide
  source 1..23: mol_type = other RNA
aaacatgaggattacccatgtcg

SEQ ID NO: 20 - RNA, 23 nt, synthetic construct
  misc_feature 1..23: note = Description of Artificial Sequence: Synthetic binding oligonucleotide
  source 1..23: mol_type = other RNA
aaacatgaggatcacccatgtcg

SEQ ID NO: 21 - RNA, 15 nt, synthetic construct
  misc_feature 1..15: note = Description of Artificial Sequence: Synthetic binding oligonucleotide
  source 1..15: mol_type = other RNA
gccctgaagaagggc

SEQ ID NO: 22 - RNA, 15 nt, synthetic construct
  misc_feature 1..15: note = Description of Artificial Sequence: Synthetic binding oligonucleotide
  source 1..15: mol_type = other RNA
gccctgaaaaagggc

SEQ ID NO: 23 - RNA, 20 nt, synthetic construct
  misc_feature 1..20: note = Description of Artificial Sequence: Synthetic binding oligonucleotide
  source 1..20: mol_type = other RNA
gttcactgccgtataggcag

SEQ ID NO: 24 - RNA, 28 nt, synthetic construct
  misc_feature 1..28: note = Description of Artificial Sequence: Synthetic binding oligonucleotide
  source 1..28: mol_type = other RNA
gttcactgccgtataggcagctaagaaa

SEQ ID NO: 25 - protein, 130 aa, unidentified
  REGION 1..130: note = Enterobacteria phage MS2
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 26 - protein, 130 aa, unidentified
  REGION 1..130: note = Enterobacteria phage MS2
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 27 - protein, 117 aa, unidentified
  REGION 1..117: note = Enterobacteria phage MS2
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 28 - protein, 262 aa, synthetic construct
  REGION 1..262: note = Description of Artificial Sequence: Synthetic dimeric MS2 coat polypeptide
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGLYGAMASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSLIN

SEQ ID NO: 29 - protein, 262 aa, synthetic construct
  REGION 1..262: note = Description of Artificial Sequence: Synthetic dimeric MS2 N55K polypeptide
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGLYGAMASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSLIN

SEQ ID NO: 30 - protein, 236 aa, synthetic construct
  REGION 1..236: note = Description of Artificial Sequence: Synthetic dimeric MS2deltaFG polypeptide
MASNFTQFVLVDNGGTGDVTVAPSNFANGVAEWISSNSRSQAYKVTCSVRQSSAQKRKYTIKVEVPKGAWRSYLNMELTIPIFATNSDCELIVKAMQGLLKDGNPIPSAIAANSGLYGAMASNFTQFVLVDNGGTGDVTVAPSNFANGVAEWISSNSRSQAYKVTCSVRQSSAQKRKYTIKVEVPKGAWRSYLNMELTIPIFATNSDCELIVKAMQGLLKDGNPIPSAIAANSLIN

SEQ ID NO: 31 - protein, 22 aa, Enterobacteria phage lambda
MDAQTRRRERRAEKQAQWKAAN

SEQ ID NO: 32 - protein, 107 aa, Enterobacteria phage lambda
MDAQTRRRERRAEKQAQWKAANPLLVGVSAKPVNRPILSLNRKPKSRVESALNPIDLTVLAEYHKQIESNLQRIERKNQRTWYSKPGERGITCSGRQKIKGKSIPLI

SEQ ID NO: 33 - protein, 583 aa, Planomicrobium okeanokoites
MFLSMVSKIRTFGWVQNPGKFENLKRVVQVFDRNSKVHNEVKNIKIPTLVKESKIQKELVAIMNQHDLIYTYKELVGTGTSIRSEAPCDAIIQATIADQGNKKGYIDNWSSDGFLRWAHALGFIEYINKSDSFVITDVGLAYSKSADGSAIEKEILIEAISSYPPAIRILTLLEDGQHLTKFDLGKNLGFSGESGFTSLPEGILLDTLANAMPKDKGEIRNNWEGSSDKYARMIGGWLDKLGLVKQGKKEFIIPTLGKPDNKEFISHAFKITGEGLKVLRRAKGSTKFTRVPKRVYWEMLATNLTDKEYVRTRRALILEILIKAGSLKIEQIQDNLKKLGFDEVIETIENDIKGLINTGIFIEIKGRFYQLKDHILQFVIPNRGVTKQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 34 - DNA, 1752 bp, Planomicrobium okeanokoites
  source 1..1752: mol_type = genomic DNA
atgtttttgagtatggtttctaaaataagaactttcggttgggttcaaaatccaggtaaatttgagaatttaaaacgagtagttcaagtatttgatagaaattctaaagtacataatgaagtgaaaaatataaagataccaaccctagtcaaagaaagtaagatccaaaaagaactagttgctattatgaatcaacatgatttgatttatacatataaagagttagtaggaacaggaacttcaatacgttcagaagcaccatgcgatgcaattattcaagcaacaatagcagatcaaggaaataaaaaaggctatatcgataattggtcatctgacggttttttgcgttgggcacatgctttaggatttattgaatatataaataaaagtgattcttttgtaataactgatgttggacttgcttactctaaatcagctgacggcagcgccattgaaaaagagattttgattgaagcgatatcatcttatcctccagcgattcgtattttaactttgctagaagatggacaacatttgacaaagtttgatcttggcaagaatttaggttttagtggagaaagtggatttacttctctaccggaaggaattcttttagatactctagctaatgctatgcctaaagataaaggcgaaattcgtaataattgggaaggatcttcagataagtacgcaagaatgataggtggttggctggataaactaggattagtaaagcaaggaaaaaaagaatttatcattcctactttgggtaagccggacaataaagagtttatatcccacgcttttaaaattactggagaaggtttgaaagtactgcgtcgagcaaaaggctctacaaaatttacacgtgtacctaaaagagtatattgggaaatgcttgctacaaacctaaccgataaagagtatgtaagaacaagaagagctttgattttagaaatattaatcaaagctggatcattaaaaatagaacaaatacaagacaacttgaagaaattaggatttgatgaagttatagaaactattgaaaatgatatcaaaggcttaattaacacaggtatatttatagaaatcaaagggcgattttatcaattgaaagaccatattcttcaatttgtaatacctaatcgtggtgtgactaagcaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtgagacggaaatttaataacggcgagataaacttttaa

SEQ ID NO: 35 - DNA, 16 bp, synthetic construct
  misc_feature 1..16: note = Description of Artificial Sequence: Synthetic primer
  source 1..16: mol_type = other DNA
gtaaaacgacggccag

SEQ ID NO: 36 - RNA, 28 nt, synthetic construct
  misc_feature 1..28: note = Description of Artificial Sequence: Synthetic Csy4 RNA binding oligonucleotide
  source 1..28: mol_type = other RNA
gttcactgccgtataggcagctaagaaa

SEQ ID NO: 37 - RNA, 252 nt, synthetic construct
  misc_feature 1..252: note = Description of Artificial Sequence: Synthetic multiplex guide RNA polynucleotide
  source 1..252: mol_type = other RNA
gttcactgccgtataggcagnnnnnnnnnnnnnnnnnnnngttttagagctagaaatagcaagttaaaataaggctagtccgttatcaacttgaaaaagtggcaccgagtcggtgcgttcactgccgtataggcagnnnnnnnnnnnnnnnnnnnngttttagagctagaaatagcaagttaaaataaggctagtccgttatcaacttgaaaaagtggcaccgagtcggtgcgttcactgccgtataggcag

SEQ ID NO: 38 - DNA, 1167 bp, synthetic construct
  misc_feature 1..1167: note = Description of Artificial Sequence: Synthetic Csy4-FokI N-terminal fusion polynucleotide
  source 1..1167: mol_type = other DNA
atggaccactacctcgacattcgcttgcgaccggacccggaatttcccccggcgcaactcatgagcgtgctcttcggcaagctccaccaggccctggtggcacagggcggggacaggatcggcgtgagcttccccgacctcgacgaaagccgctcccggctgggcgagcgcctgcgcattcatgcctcggcggacgaccttcgtgccctgctcgcccggccctggctggaagggttgcgggaccatctgcaattcggagaaccggcagtcgtgcctcaccccacaccgtaccgtcaggtcagtcgggttcaggcgaaaagcaatccggaacgcctgcggcggcggctcatgcgccggcacgatctgagtgaggaggaggctcggaaacgcattcccgatacggtcgcgagagccttggacctgcccttcgtcacgctacgcagccagagcaccggacagcacttccgtctcttcatccgccacgggccgttgcaggtgacggcagaggaaggaggattcacctgttacgggttgagcaaaggaggtttcgttccctggttcggtggcggtggatcccaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtcagacggaaatttaataacggcgagataaacttttga

SEQ ID NO: 39 - protein, 388 aa, synthetic construct
  REGION 1..388: note = Description of Artificial Sequence: Synthetic Csy4-FokI N-terminal fusion polypeptide
MDHYLDIRLRPDPEFPPAQLMSVLFGKLHQALVAQGGDRIGVSFPDLDESRSRLGERLRIHASADDLRALLARPWLEGLRDHLQFGEPAVVPHPTPYRQVSRVQAKSNPERLRRRLMRRHDLSEEEARKRIPDTVARALDLPFVTLRSQSTGQHFRLFIRHGPLQVTAEEGGFTCYGLSKGGFVPWFGGGGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 40 - DNA, 1167 bp, synthetic construct
  misc_feature 1..1167: note = Description of Artificial Sequence: Synthetic Csy4-FokI C-terminal fusion polynucleotide
  source 1..1167: mol_type = other DNA
atgcaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtcagacggaaatttaataacggcgagataaactttggtggcggtggatccgaccactacctcgacattcgcttgcgaccggacccggaatttcccccggcgcaactcatgagcgtgctcttcggcaagctccaccaggccctggtggcacagggcggggacaggatcggcgtgagcttccccgacctcgacgaaagccgctcccggctgggcgagcgcctgcgcattcatgcctcggcggacgaccttcgtgccctgctcgcccggccctggctggaagggttgcgggaccatctgcaattcggagaaccggcagtcgtgcctcaccccacaccgtaccgtcaggtcagtcgggttcaggcgaaaagcaatccggaacgcctgcggcggcggctcatgcgccggcacgatctgagtgaggaggaggctcggaaacgcattcccgatacggtcgcgagagccttggacctgcccttcgtcacgctacgcagccagagcaccggacagcacttccgtctcttcatccgccacgggccgttgcaggtgacggcagaggaaggaggattcacctgttacgggttgagcaaaggaggtttcgttccctggttctga

SEQ ID NO: 41 - protein, 388 aa, synthetic construct
  REGION 1..388: note = Description of Artificial Sequence: Synthetic Csy4-FokI C-terminal fusion polypeptide
MQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFGGGGSDHYLDIRLRPDPEFPPAQLMSVLFGKLHQALVAQGGDRIGVSFPDLDESRSRLGERLRIHASADDLRALLARPWLEGLRDHLQFGEPAVVPHPTPYRQVSRVQAKSNPERLRRRLMRRHDLSEEEARKRIPDTVARALDLPFVTLRSQSTGQHFRLFIRHGPLQVTAEEGGFTCYGLSKGGFVPWF

SEQ ID NO: 42 - RNA, 79 nt, synthetic construct
  misc_feature 1..79: note = Description of Artificial Sequence: Synthetic tracrRNA oligonucleotide
  source 1..79: mol_type = other RNA
ggaaccattcaaaacagcatagcaagttaaaataaggctagtccgttatcaacttgaaaaagtggcaccgagtcggtgc

SEQ ID NO: 43 - RNA, 70 nt, synthetic construct
  misc_feature 1..70: note = Description of Artificial Sequence: Synthetic tracrRNA oligonucleotide
  source 1..70: mol_type = other RNA
caaaacagcatagcaagttaaaataaggctagtccgttatcaacttgaaaaagtggcaccgagtcggtgc

SEQ ID NO: 44 - RNA, 45 nt, synthetic construct
  misc_feature 1..45: note = Description of Artificial Sequence: Synthetic tracrRNA oligonucleotide
  source 1..45: mol_type = other RNA
tagcaagttaaaataaggctagtccgttatcaacttgaaaaagtg

SEQ ID NO: 45 - RNA, 32 nt, synthetic construct
  misc_feature 1..32: note = Description of Artificial Sequence: Synthetic tracrRNA oligonucleotide
  source 1..32: mol_type = other RNA
tagcaagttaaaataaggctagtccgttatca

SEQ ID NO: 46 - RNA, 26 nt, synthetic construct
  misc_feature 1..26: note = Description of Artificial Sequence: Synthetic tracrRNA oligonucleotide
  source 1..26: mol_type = other RNA
tagcaagttaaaataaggctagtccg

SEQ ID NO: 47 - protein, 4 aa, synthetic construct
  REGION 1..4: note = Description of Artificial Sequence: Synthetic linker peptide
GGGS

SEQ ID NO: 48 - protein, 5 aa, synthetic construct
  REGION 1..5: note = Description of Artificial Sequence: Synthetic linker peptide
GGGGS

SEQ ID NO: 49 - DNA, 23 bp, synthetic construct
  misc_feature 1..23: note = Description of Artificial Sequence: Synthetic target binding site oligonucleotide
  source 1..23: mol_type = other DNA
ggtgagtgagtgtgtgcgtgtgg

SEQ ID NO: 50 - DNA, 18 bp, synthetic construct
  misc_feature 1..18: note = Description of Artificial Sequence: Synthetic target binding site oligonucleotide
  source 1..18: mol_type = other DNA
gggcgatgccacctacgg

SEQ ID NO: 51 - DNA, 20 bp, synthetic construct
  misc_feature 1..20: note = Description of Artificial Sequence: Synthetic target binding site oligonucleotide
  source 1..20: mol_type = other DNA
gagggcgatgccacctacgg

SEQ ID NO: 52 - DNA, 22 bp, synthetic construct
  misc_feature 1..22: note = Description of Artificial Sequence: Synthetic target binding site oligonucleotide
  source 1..22: mol_type = other DNA
gcgagggcgatgccacctacgg

SEQ ID NO: 53 - DNA, 23 bp, synthetic construct
  misc_feature 1..23: note = Description of Artificial Sequence: Synthetic target binding site oligonucleotide
  source 1..23: mol_type = other DNA
ggcgagggcgatgccacctacgg

SEQ ID NO: 54 - DNA, 24 bp, synthetic construct
  misc_feature 1..24: note = Description of Artificial Sequence: Synthetic target binding site oligonucleotide
  source 1..24: mol_type = other DNA
gggcgagggcgatgccacctacgg

SEQ ID NO: 55 - DNA, 26 bp, synthetic construct
  misc_feature 1..26: note = Description of Artificial Sequence: Synthetic target binding site oligonucleotide
  source 1..26: mol_type = other DNA
gagggcgagggcgatgccacctacgg

SEQ ID NO: 56 - DNA, 28 bp, synthetic construct
  misc_feature 1..28: note = Description of Artificial Sequence: Synthetic target binding site oligonucleotide
  source 1..28: mol_type = other DNA
gcgagggcgagggcgatgccacctacgg

SEQ ID NO: 57 - DNA, 21 bp, synthetic construct
  misc_feature 1..21: note = Description of Artificial Sequence: Synthetic target binding site oligonucleotide
  source 1..21: mol_type = other DNA
gcacgggcagcttgccggtgg

SEQ ID NO: 58 - DNA, 23 bp, synthetic construct
  misc_feature 1..23: note = Description of Artificial Sequence: Synthetic target binding site oligonucleotide
  source 1..23: mol_type = other DNA
gggcacgggcagcttgccggtgg

SEQ ID NO: 59 - DNA, 20 bp, synthetic construct
  misc_feature 1..20: note = Description of Artificial Sequence: Synthetic target binding site oligonucleotide
  source 1..20: mol_type = other DNA
gccgttcttctgcttgtcgg

SEQ ID NO: 60 - DNA, 23 bp, synthetic construct
  misc_feature 1..23: note = Description of Artificial Sequence: Synthetic target binding site oligonucleotide
  source 1..23: mol_type = other DNA
gatgccgttcttctgcttgtcgg

SEQ ID NO: 61 - DNA, 19 bp, synthetic construct
  misc_feature 1..19: note = Description of Artificial Sequence: Synthetic target binding site oligonucleotide
  source 1..19: mol_type = other DNA
gtgcagatgaacttcaggg

SEQ ID NO: 62 - DNA, 20 bp, synthetic construct
  misc_feature 1..20: note = Description of Artificial Sequence: Synthetic target binding site oligonucleotide
  source 1..20: mol_type = other DNA
ggtgcagatgaacttcaggg

SEQ ID NO: 63 - DNA, 23 bp, synthetic construct
  misc_feature 1..23: note = Description of Artificial Sequence: Synthetic target binding site oligonucleotide
  source 1..23: mol_type = other DNA
ggtggtgcagatgaacttcaggg

SEQ ID NO: 64 - DNA, 19 bp, synthetic construct
  misc_feature 1..19: note = Description of Artificial Sequence: Synthetic target binding site oligonucleotide
  source 1..19: mol_type = other DNA
gaggagctgttcaccgggg

SEQ ID NO: 65 - DNA, 21 bp, synthetic construct
  misc_feature 1..21: note = Description of Artificial Sequence: Synthetic target binding site oligonucleotide
  source 1..21: mol_type = other DNA
gcgaggagctgttcaccgggg

SEQ ID NO: 66 - DNA, 23 bp, synthetic construct
  misc_feature 1..23: note = Description of Artificial Sequence: Synthetic target binding site oligonucleotide
  source 1..23: mol_type = other DNA
gggcgaggagctgttcaccgggg

SEQ ID NO: 67 - DNA, 21 bp, synthetic construct
  misc_feature 1..21: note = Description of Artificial Sequence: Synthetic target binding site oligonucleotide
  source 1..21: mol_type = other DNA
gtggggggagtttgctcctgg

SEQ ID NO: 68 - DNA, 23 bp, synthetic construct
  misc_feature 1..23: note = Description of Artificial Sequence: Synthetic target binding site oligonucleotide
  source 1..23: mol_type = other DNA
gggtggggggagtttgctcctgg

SEQ ID NO: 69 - DNA, 20 bp, synthetic construct
  misc_feature 1..20: note = Description of Artificial Sequence: Synthetic target binding site oligonucleotide
  source 1..20: mol_type = other DNA
gagtgagtgtgtgcgtgtgg

SEQ ID NO: 70 - DNA, 23 bp, synthetic construct
  misc_feature 1..23: note = Description of Artificial Sequence: Synthetic target binding site oligonucleotide
  source 1..23: mol_type = other DNA
ggtgagtgagtgtgtgcgtgtgg

SEQ ID NO: 71 - DNA, 21 bp, synthetic construct
  misc_feature 1..21: note = Description of Artificial Sequence: Synthetic target binding site oligonucleotide
  source 1..21: mol_type = other DNA
gtccgagcagaagaagaaggg

SEQ ID NO: 72 - DNA, 23 bp, synthetic construct
  misc_feature 1..23: note = Description of Artificial Sequence: Synthetic target binding site oligonucleotide
  source 1..23: mol_type = other DNA
gagtccgagcagaagaagaaggg

SEQ ID NO: 73 - DNA, 20 bp, synthetic construct
  misc_feature 1..20: note = Description of Artificial Sequence: Synthetic target binding site oligonucleotide
  source 1..20: mol_type = other DNA
gatgtagtgtttccacaggg

SEQ ID NO: 74 - DNA, 23 bp, synthetic construct
  misc_feature 1..23: note = Description of Artificial Sequence: Synthetic target binding site oligonucleotide
  source 1..23: mol_type = other DNA
gcagatgtagtgtttccacaggg

SEQ ID NO: 75 - DNA, 67 bp, Homo sapiens
  source 1..67: mol_type = genomic DNA
gaagctggaggaggaagggcctgagtccgagcagaagaagaagggctcccatcacatcaaccggtgg

SEQ ID NO: 76 - DNA, 15 bp, synthetic construct
  misc_feature 1..15: note = Description of Artificial Sequence: Synthetic oligonucleotide
  source 1..15: mol_type = other DNA
gaagctggaggagga

SEQ ID NO: 77 - DNA, 11 bp, synthetic construct
  misc_feature 1..11: note = Description of Artificial Sequence: Synthetic oligonucleotide
  source 1..11: mol_type = other DNA
tcaaccggtgg

SEQ ID NO: 78 - DNA, 18 bp, synthetic construct
  misc_feature 1..18: note = Description of Artificial Sequence: Synthetic oligonucleotide
  source 1..18: mol_type = other DNA
gaagctggaggaggaagg

SEQ ID NO: 79 - DNA, 25 bp, synthetic construct
  misc_feature 1..25: note = Description of Artificial Sequence: Synthetic oligonucleotide
  source 1..25: mol_type = other DNA
gggctcccatcacatcaaccggtgg

SEQ ID NO: 80 - DNA, 24 bp, synthetic construct
  misc_feature 1..24: note = Description of Artificial Sequence: Synthetic oligonucleotide
  source 1..24: mol_type = other DNA
gaagctggaggaggaagggcctga

SEQ ID NO: 81 - DNA, 14 bp, synthetic construct
  misc_feature 1..14: note = Description of Artificial Sequence: Synthetic oligonucleotide
  source 1..14: mol_type = other DNA
gaagctggaggagg

SEQ ID NO: 82 - DNA, 39 bp, synthetic construct
  misc_feature 1..39: note = Description of Artificial Sequence: Synthetic oligonucleotide
  source 1..39: mol_type = other DNA
gaagctggaggaggaagggcccatcacatcaaccggtgg

SEQ ID NO: 83 - DNA, 40 bp, synthetic construct
  misc_feature 1..40: note = Description of Artificial Sequence: Synthetic oligonucleotide
  source 1..40: mol_type = other DNA
gaagctggaggaggaagggctcgcacacatcaaccggtgg

SEQ ID NO: 84 - DNA, 42 bp, synthetic construct
  misc_feature 1..42: note = Description of Artificial Sequence: Synthetic oligonucleotide
  source 1..42: mol_type = other DNA
gaagctggaggaggaagggccttccatcacatcaaccggtgg

SEQ ID NO: 85 - DNA, 46 bp, synthetic construct
  misc_feature 1..46: note = Description of Artificial Sequence: Synthetic oligonucleotide
  source 1..46: mol_type = other DNA
gaagctggaggaggaagggcctgagtcccatcacatcaaccggtgg

SEQ ID NO: 86 - DNA, 52 bp, synthetic construct
  misc_feature 1..52: note = Description of Artificial Sequence: Synthetic oligonucleotide
  source 1..52: mol_type = other DNA
gaagctggaggaggaagggcctgagtccgagtcccatcacatcaaccggtgg

SEQ ID NO: 87 - DNA, 58 bp, synthetic construct
  misc_feature 1..58: note = Description of Artificial Sequence: Synthetic oligonucleotide
  source 1..58: mol_type = other DNA
gaagctggaggaggaagggcctgagtccgagcagaagtcccatcacatcaaccggtgg

SEQ ID NO: 88 - DNA, 59 bp, synthetic construct
  misc_feature 1..59: note = Description of Artificial Sequence: Synthetic oligonucleotide
  source 1..59: mol_type = other DNA
gaagctggaggaggaagggcctgagtcctgccgtttgtagccatcacatcaaccggtgg

SEQ ID NO: 89 - DNA, 61 bp, synthetic construct
  misc_feature 1..61: note = Description of Artificial Sequence: Synthetic oligonucleotide
  source 1..61: mol_type = other DNA
gaagctggaggaggaagggcctgagtccgagcagaagagctcccatcacatcaaccggtgg

SEQ ID NO: 90 - DNA, 61 bp, synthetic construct
  misc_feature 1..61: note = Description of Artificial Sequence: Synthetic oligonucleotide
  source 1..61: mol_type = other DNA
gaagctggaggaggaagggcctgagtccgagcagaagaactcccatcacatcaaccggtgg

SEQ ID NO: 91 - DNA, 64 bp, synthetic construct
  misc_feature 1..64: note = Description of Artificial Sequence: Synthetic oligonucleotide
  source 1..64: mol_type = other DNA
gaagctggaggaggaagggcctgagtccgagcagaagaagggctcccatcacatcaaccggtgg

SEQ ID NO: 92 - DNA, 65 bp, synthetic construct
  misc_feature 1..65: note = Description of Artificial Sequence: Synthetic oligonucleotide
  source 1..65: mol_type = other DNA
gaagctggaggaggaagggcctgagtccgagcagaagaaagggctcccatcacatcaaccggtgg

SEQ ID NO: 93 - DNA, 67 bp, synthetic construct
  misc_feature 1..67: note = Description of Artificial Sequence: Synthetic oligonucleotide
  source 1..67: mol_type = other DNA
gaagctggaggaggaagggcctgagtccgagcagaagaacagaagggctcccatcacatcaaccggt

SEQ ID NO: 94 - DNA, 67 bp, Homo sapiens; identical to SEQ ID NO: 75
  source 1..67: mol_type = genomic DNA

SEQ ID NO: 95 - DNA, 31 bp, synthetic construct
  misc_feature 1..31: note = Description of Artificial Sequence: Synthetic oligonucleotide
  source 1..31: mol_type = other DNA
gaagctggaggaggaagggcctgagtccgag

SEQ ID NO: 96 - DNA, 11 bp, synthetic construct
  misc_feature 1..11: note = Description of Artificial Sequence: Synthetic oligonucleotide
  source 1..11: mol_type = other DNA
gaagctggagg

SEQ ID NO: 97 - DNA, 11 bp, synthetic construct
  misc_feature 1..11: note = Description of Artificial Sequence: Synthetic oligonucleotide
  source 1..11: mol_type = other DNA
gaagctggagg

SEQ ID NO: 98 - DNA, 28 bp, synthetic construct
  misc_feature 1..28: note = Description of Artificial Sequence: Synthetic oligonucleotide
  source 1..28: mol_type = other DNA
gaagctggaggaggaagggcctgagtgg

SEQ ID NO: 99 - DNA, 41 bp, synthetic construct
  misc_feature 1..41: note = Description of Artificial Sequence: Synthetic oligonucleotide
  source 1..41: mol_type = other DNA
gaagctggaggaggaagggctcccatcacatcaaccggtgg

SEQ ID NO: 100 - DNA, 45 bp, synthetic construct
  misc_feature 1..45: note = Description of Artificial Sequence: Synthetic oligonucleotide
  source 1..45: mol_type = other DNA
gaagctggaggaggaagggcctgagtccatcacatcaaccggtgg

SEQ ID NO: 101 - DNA, 46 bp, synthetic construct; identical to SEQ ID NO: 85
  misc_feature 1..46: note = Description of Artificial Sequence: Synthetic oligonucleotide
  source 1..46: mol_type = other DNA

SEQ ID NO: 102 - DNA, 49 bp, synthetic construct
  misc_feature 1..49: note = Description of Artificial Sequence: Synthetic oligonucleotide
  source 1..49: mol_type = other DNA
gaagctggaggaggaagggcctgagtccgagcatcacatcaaccggtgg

SEQ ID NO: 103 - DNA, 53 bp, synthetic construct
  misc_feature 1..53: note = Description of Artificial Sequence: Synthetic oligonucleotide
  source 1..53: mol_type = other DNA
gaagctggaggaggaagggcctgagtccgagctcccatcacatcaaccggtgg

SEQ ID NO: 104 - DNA, 61 bp, synthetic construct
  misc_feature 1..61: note = Description of Artificial Sequence: Synthetic oligonucleotide
  source 1..61: mol_type = other DNA
gaagctggaggaggaagggcctgagtccgagcagaagggctcccatcacatcaaccggtgg

SEQ ID NO: 105 - DNA, 64 bp, synthetic construct; identical to SEQ ID NO: 91
  misc_feature 1..64: note = Description of Artificial Sequence: Synthetic oligonucleotide
  source 1..64: mol_type = other DNA

SEQ ID NO: 106 - DNA, 65 bp, synthetic construct
  misc_feature 1..65: note = Description of Artificial Sequence: Synthetic oligonucleotide
  source 1..65: mol_type = other DNA
gaagctggaggaggaagggcctgagtccgagcagaaagaagggctcccatcacatcaaccggtgg

SEQ ID NO: 107 - DNA, 67 bp, synthetic construct; identical to SEQ ID NO: 93
  misc_feature 1..67: note = Description of Artificial Sequence: Synthetic oligonucleotide
  source 1..67: mol_type = other DNA

SEQ ID NO: 108 - DNA, 23 bp, synthetic construct
  misc_feature 1..23: note = Synthetic target binding site oligonucleotide
  source 1..23: mol_type = other DNA
ccacacgcacacactcactcacc

SEQ ID NO: 109 - RNA, 20 nt, synthetic construct
  misc_feature 1..20: note = Synthetic target binding site oligonucleotide
  source 1..20: mol_type = other RNA
ggtgagtgagtgtgtgcgtg

SEQ ID NO: 110 - DNA, 20 bp, synthetic construct
  misc_feature 1..20: note = Synthetic target binding site oligonucleotide
  source 1..20: mol_type = other DNA
gtttgtactttgtcctccgg

SEQ ID NO: 111 - DNA, 23 bp, synthetic construct
  misc_feature 1..23: note = Synthetic target binding site oligonucleotide
  source 1..23: mol_type = other DNA
gccgtttgtactttgtcctccgg

SEQ ID NO: 112 - DNA, 21 bp, synthetic construct
  misc_feature 1..21: note = Synthetic target binding site oligonucleotide
  source 1..21: mol_type = other DNA
gaagactgaggctacataggg

SEQ ID NO: 113 - DNA, 23 bp, synthetic construct
  misc_feature 1..23: note = Synthetic target binding site oligonucleotide
  source 1..23: mol_type = other DNA
gggaagactgaggctacataggg

SEQ ID NO: 114 - DNA, 20 bp, synthetic construct
  misc_feature 1..20: note = Synthetic target binding site oligonucleotide
  source 1..20: mol_type = other DNA
gcccccagagcagccactgg

SEQ ID NO: 115 - DNA, 23 bp, synthetic construct
  misc_feature 1..23: note = Synthetic target binding site oligonucleotide
  source 1..23: mol_type = other DNA
gaggcccccagagcagccactgg